(19) United States
(12) Patent Application Publication
(10) Pub. No.: US 2009/0054451 A1
(43) Pub. Date: Feb. 26, 2009

(54) Use of Piperidine Derivatives as Agonists of Chemokine Receptor Activity

(75) Inventors: Jacques Huck, Lyon (FR); Frederic Ooms, Hannut (BE); Christian Tyrchan, Lindome (SE); Hamid R. Hoveyda, Bruxelles (BE)

Correspondence Address: EDWARDS ANGELL PALMER & DODGE LLP, P.O. BOX 55874, BOSTON, MA, 02205, US

(73) Assignee: Euroscreen S.A., Gosselies (BE)

(21) Appl. No.: 12/185,513

(22) Filed: Aug. 4, 2008

Related U.S. Application Data
(63) Continuation of application No. PCT/EP2008/059137, filed on Jul. 11, 2008.
(60) Provisional application No. 60/959,580, filed on Jul. 13, 2007.

(30) Foreign Application Priority Data
Jul. 13, 2007 (EP) EP 07 112 467.1

(51) Int. Cl. A61K 31/496; C07D 487/04; C07D 401/02; C07D 211/08; C07D 413/02; C07D 417/02; A61K 31/519; A61K 31/506; A61K 31/4545; A61K 31/454; A61K 31/4535; A61K 31/4525; A61K 31/4468; A61P 31/18; A61P 29/00; A61P 37/02; A61P 11/06; A61P 11/00; A61P 37/08; A61P 37/06; A61P 19/02; A61P 9/10; A61P 17/06; A61P 35/00; A61P 5/00; C12N 9/99

(52) U.S. Cl. 514/253.01; 546/224; 514/329; 514/323; 546/201; 546/199; 514/326; 546/210; 546/208; 514/316; 546/191; 546/213; 546/209; 544/360; 514/318; 546/194; 514/256; 544/335; 544/263; 514/259.3; 435/238

(57) ABSTRACT

The present invention is directed to novel piperidine derivatives and to the use of piperidine derivatives of formula I

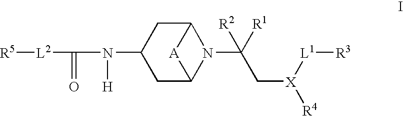

as agonists of chemokine receptor activity.

### FIELD OF THE INVENTION

The present invention relates to the use of pharmaceutically active piperidine derivatives as agonists of CC chemokine receptor activity, more specifically of CCR5 activity. Chemokines are chemotactic cytokines which play an important role in immune and inflammatory responses.

### BACKGROUND OF THE INVENTION

The Chemokine comprise a large family of proteins which have common important structural features and which have the ability to attract leukocytes. The chemokine family is divided into two main groups exhibiting characteristic structural motifs, the Cys-X-Cys (CXC) and Cys-Cys (CC) subfamilies.

CC chemokine receptors are integral membrane proteins that specifically bind and respond to cytokines of the CC chemokine family. They represent one subfamily of chemokine receptors, a large family of G protein-linked receptors that are known as seven transmembrane (7-TM) proteins since they span the cell membrane seven times. To date, ten true members of the CC chemokine receptor subfamily have been described. These are named CCR1 to CCR10 according to the IUIS/WHO Subcommittee on Chemokine Nomenclature.

Among the CC chemokine receptors, CCR5 is defined as a major co-receptor implicated in susceptibility to HIV-1 infection and disease. CCR5 is a receptor expressed on several cell types including T-lymphocytes, peripheral blood-derived dendritic cells, CD34+ hematopoietic progenitor cells and certain activated/memory Th1 lymphocytes.

Because of this well-known activity as HIV-1 co-receptor, antagonists for CCR5 have been developed with the aim of inhibiting CCR5-mediated HIV entry. The most advanced of these, Maraviroc, from Pfizer, is in good way to obtain the final FDA approval for entry on the market.

In the prior art, such as in WO0187839, for example, blocking this receptor with a CCR5 antagonist or inducing receptor internalization with a CCR5 agonist was considered of great interest to protect cells from viral infection by HIV-1.

WO0187839 describe compounds having activity as pharmaceuticals, in particular as modulators (such as agonists, partial agonists, inverse agonists or antagonists) of chemokine receptor (especially CCR5) activity, for use in the treatment of autoimmune, inflammatory, proliferative, hyperproliferative diseases, or immunologically-mediated diseases (including rejection of transplanted organs or tissues and Acquired Immunodeficiency Syndrome (AIDS)).

However, in WO0187839, as well as in all other prior art that came to the knowledge of the Inventors, it was not distinguished among the compounds which were the agonists and which were the antagonists. One goal of this invention is to distinguish CCR5 agonists from CCR5 antagonists and to disclose how agonists are structurally different from antagonists.

Without wanting to be linked by a theory, the administration of agonists only, may be advantageous in comparison with the antagonist approach because a CCR5− agonist may reduce the generation of certain types of HIV variants. Indeed, agonist molecules will promote CCR5 receptor disappearance from the cell surface by inducing its internalization. This would prevent the emergence of variants of the type able to bind the antagonist-bound CCR5, as previously observed for example with the small molecule antagonist Maraviroc (Westby M et al (2007) J Virol 81(5):2359-71). Avoiding generation of HIV variants, for example variants of the type that are able to bind the antagonist-bound CCR5, and therefore avoiding therapeutic resistance is one of the goals of this invention.

### SUMMARY OF THE INVENTION

The invention is directed to the novel piperidine derivatives of table 1, as well as to their pharmaceutically acceptable salts and solvates:

TABLE 1

| Compound | Structure |
|---|---|
| 24 | 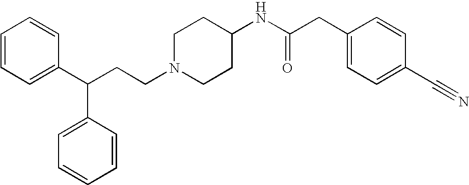 |
| 64 | 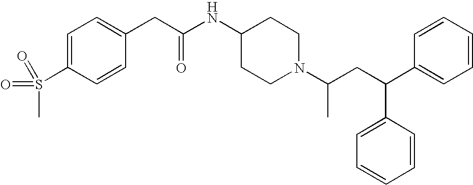 |
| 65 | 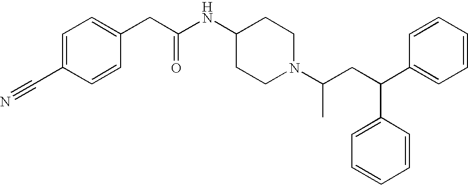 |
| 25 | 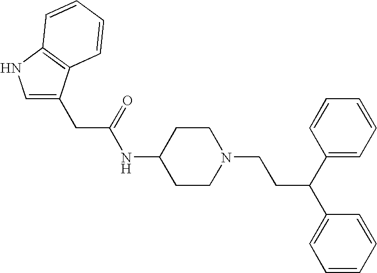 |
| 26 | 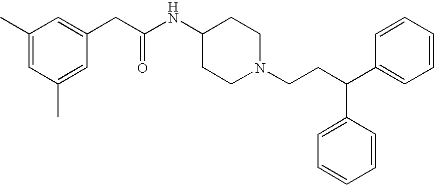 |
| 27 | 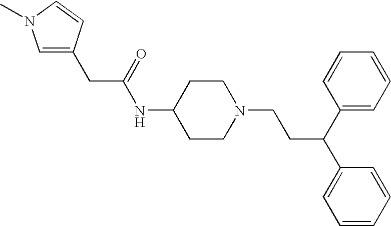 |
| 28 | 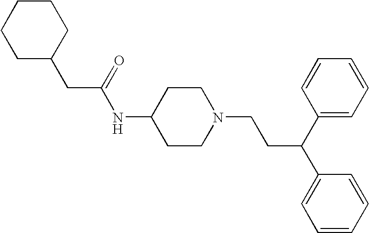 |
| 29 | 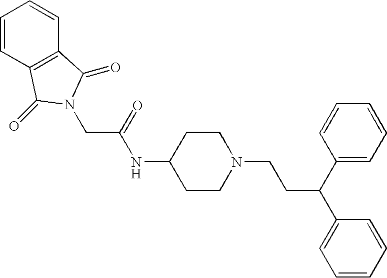 |
| 30 | 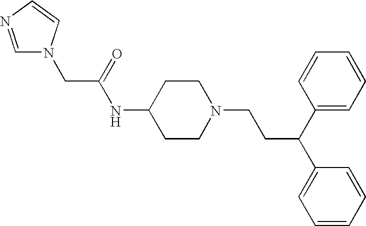 |
| 31 | 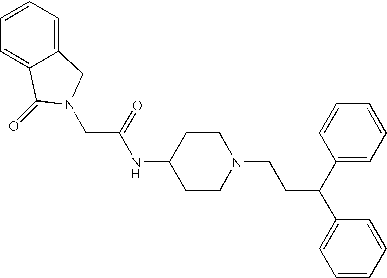 |
| 75 | 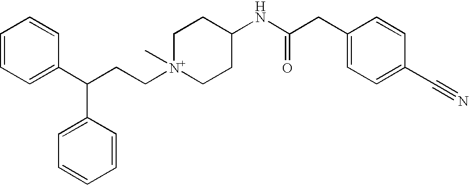 |
| 32 | 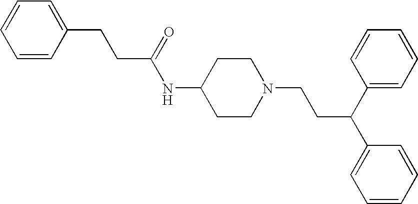 |
| 33 | 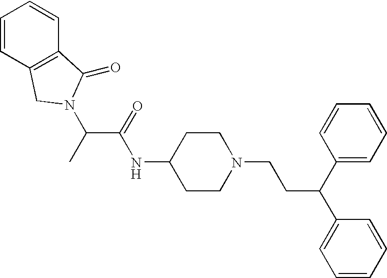 |
| 34 | 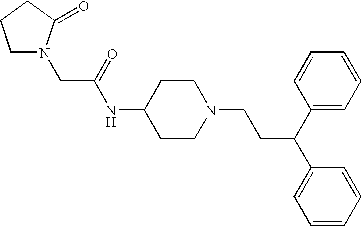 |
| 35 | 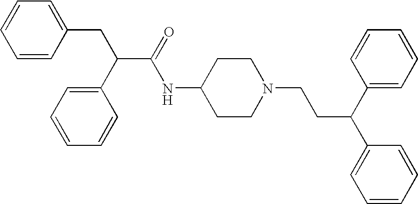 |
| 36 | 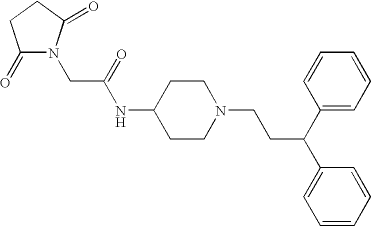 |
| 37 | 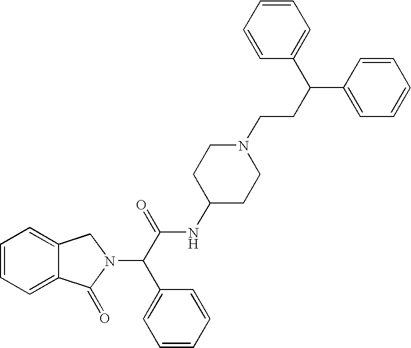 |
| 38 | 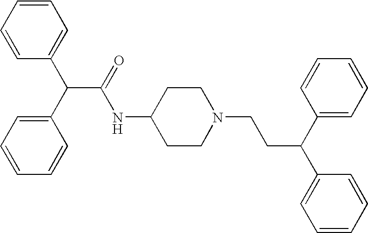 |
| 39 | 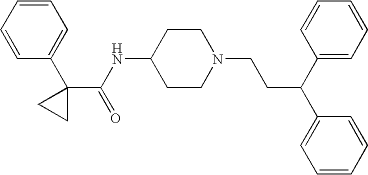 |
| 40 | 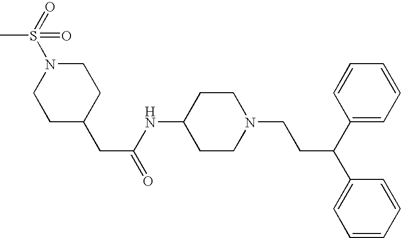 |
| 41 | 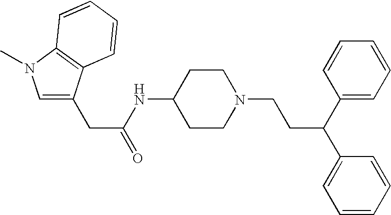 |
| 42 | 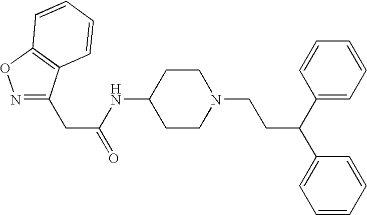 |
| 43 | 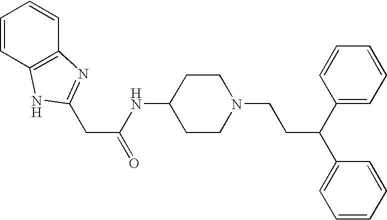 |
| 44 | 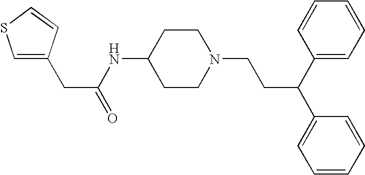 |
| 45 | 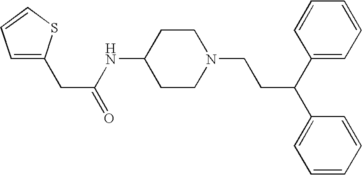 |
| 46 | 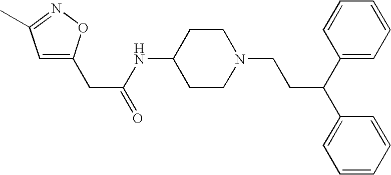 |
| 47 | 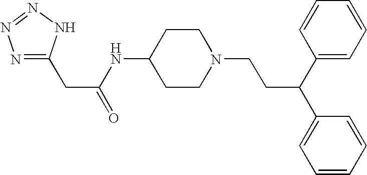 |
| 48 | 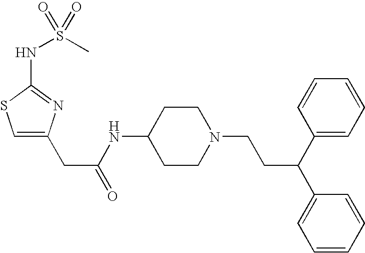 |
| 49 | 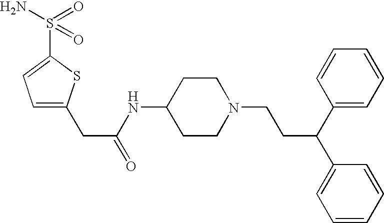 |
| 50 | 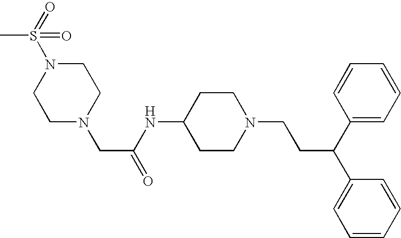 |
| 51 | 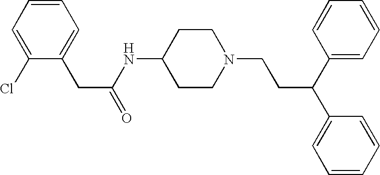 |
| 52 | 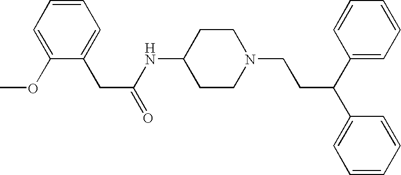 |
| 53 | 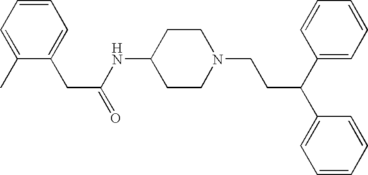 |
| 54 | 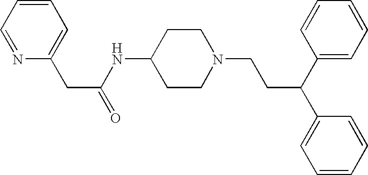 |
| 55 | 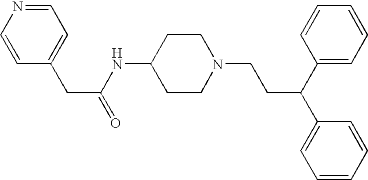 |
| 76 | 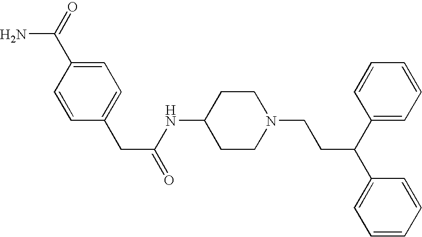 |
| 56 | 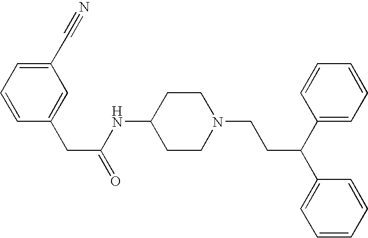 |
| 57 | 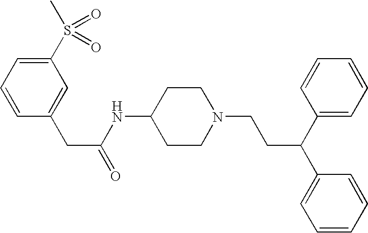 |
| 58 | 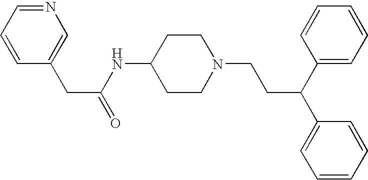 |
| 59 | 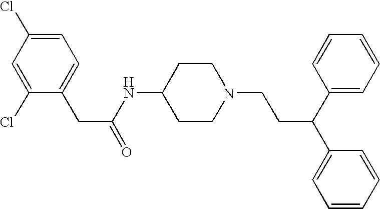 |
| 60 | 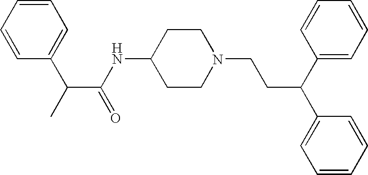 |
| 78 | 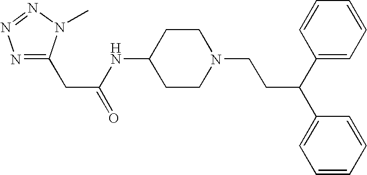 |
| 72 | 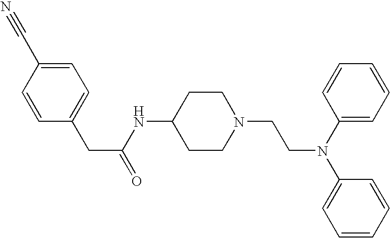 |
| 73 | 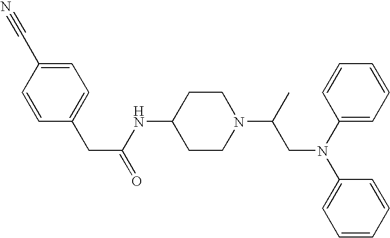 |
| 68 | 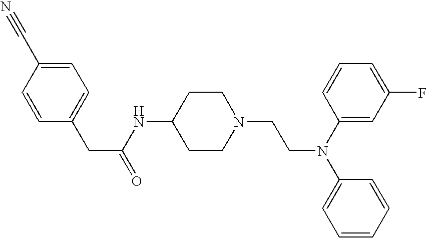 |
| 69 | 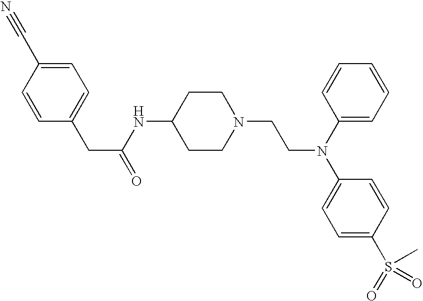 |
| 77 | 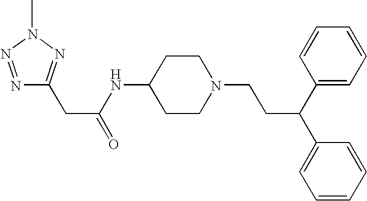 |
| 71 | 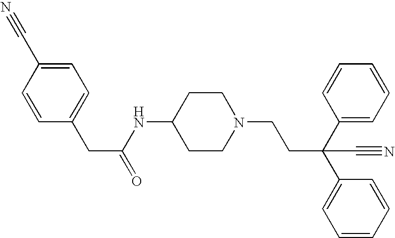 |
| 70 | 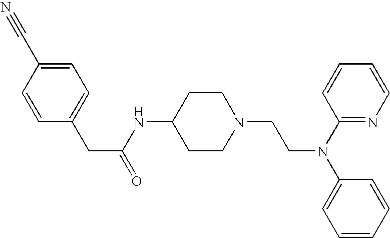 |
| 66 | 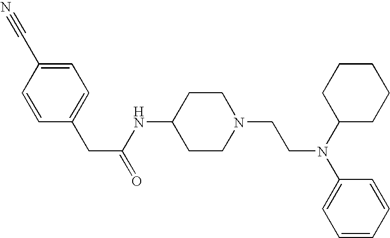 |
| 74 | 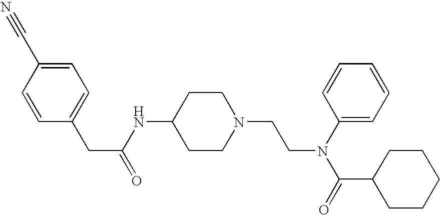 |
| 67 | 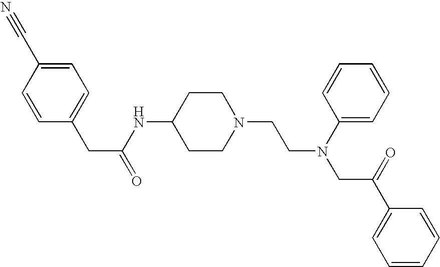 |
| 79 | 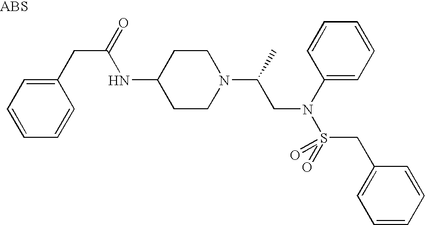 |
| 80 | 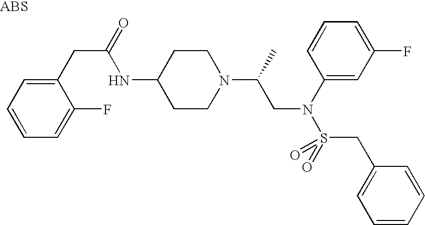 |
| 81 | 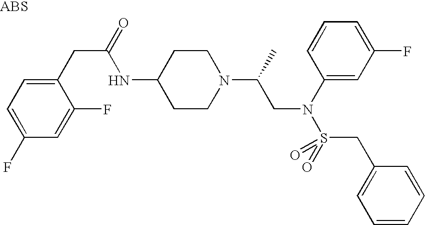 |
| 82 | 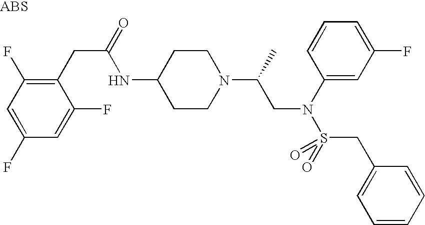 |
| 83 | 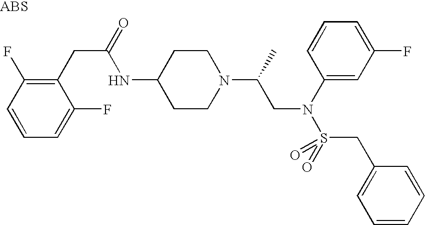 |
| 84 | 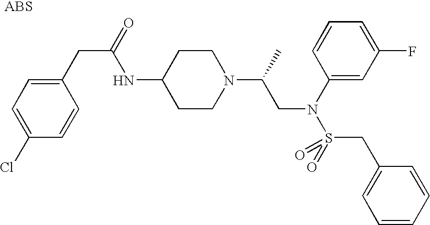 |
| 85 | 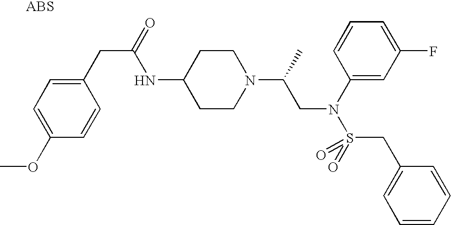 |
| 86 | 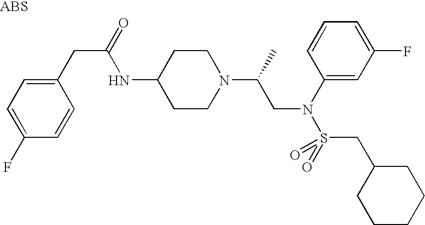 |
| 87 | 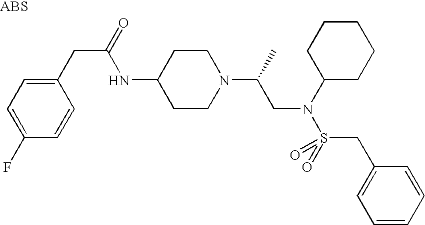 |
| 88 | 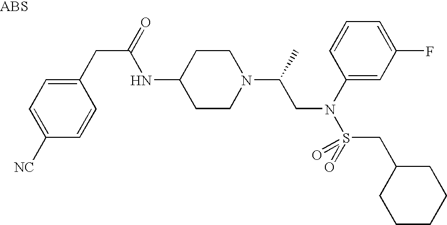 |
| 89 | 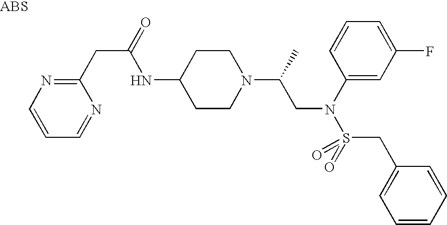 |
| 90 | 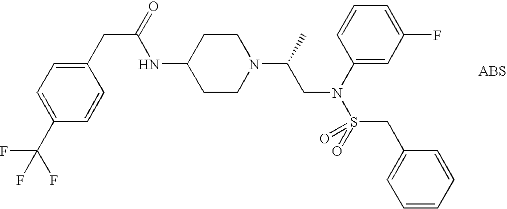 |
| 91 | 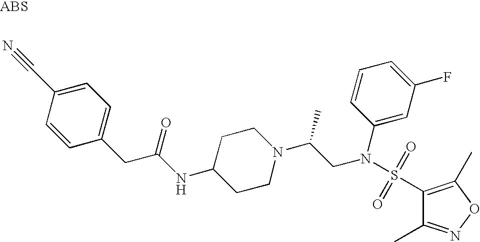 |
| 92 | 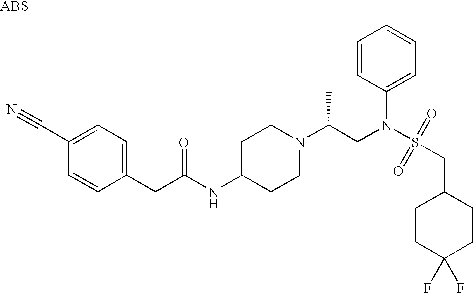 |
| 93 | 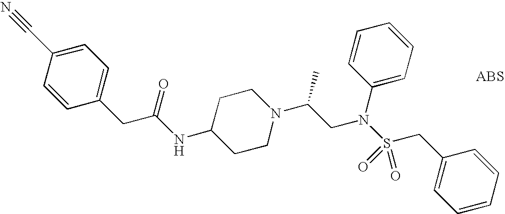 |
| 94 | 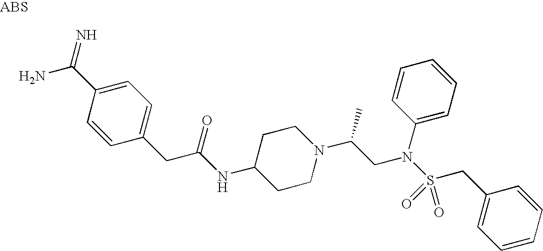 |
| 95 | 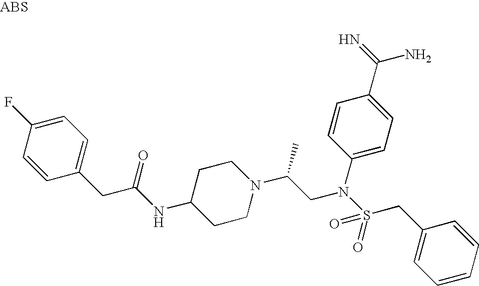 |
| 96 | 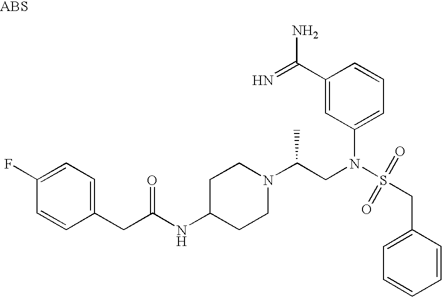 |
| 97 | 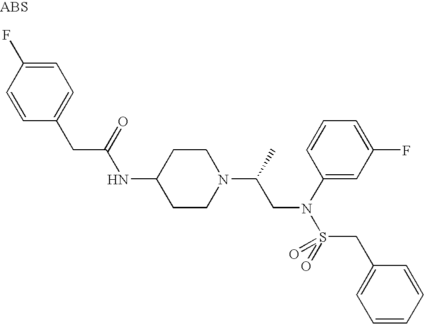 |
| 98 | 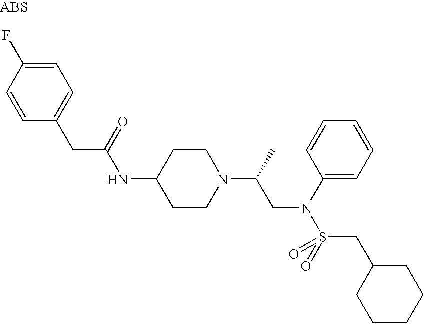 |
| 99 | 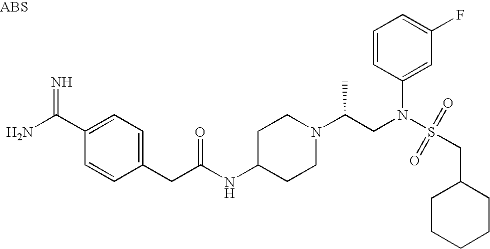 |
| 100 | 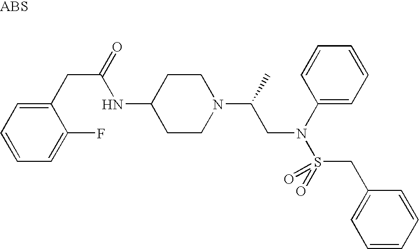 |
| 101 | 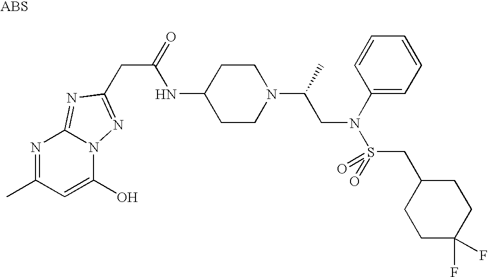 |
| 102 | 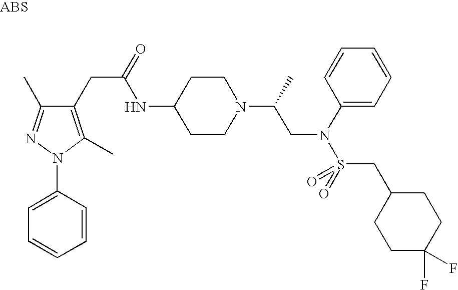 |
| 103 | 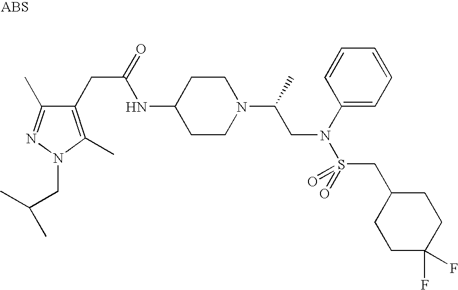 |
| 104 | 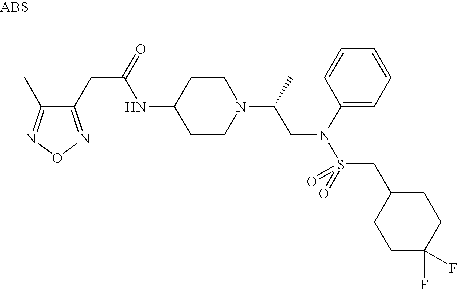 |
| 105 | 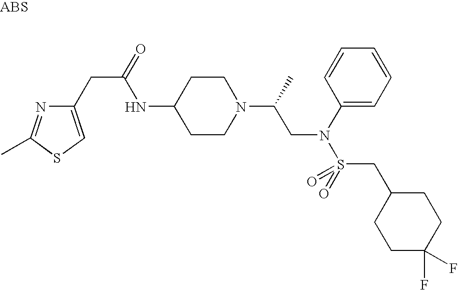 |
| 106 | 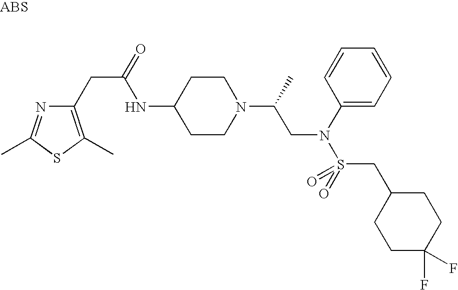 |
| 107 | 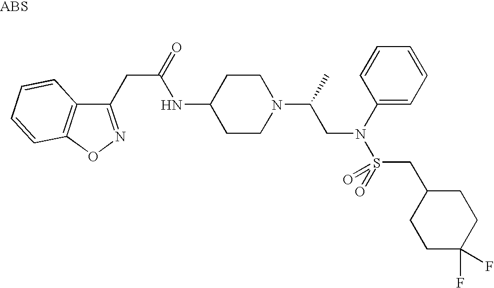 |
| 108 | 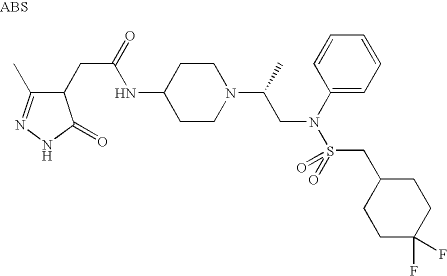 |
| 109 | 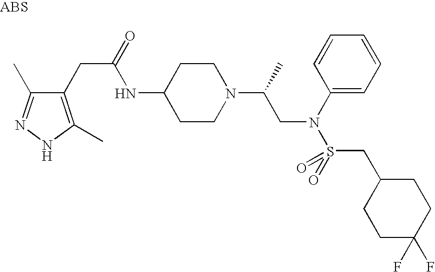 |
| 110 | 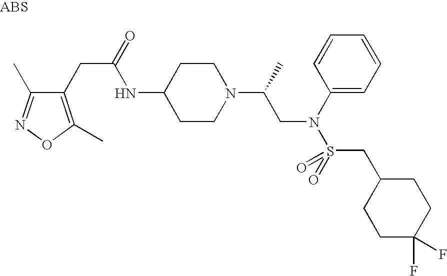 |
| 111 | 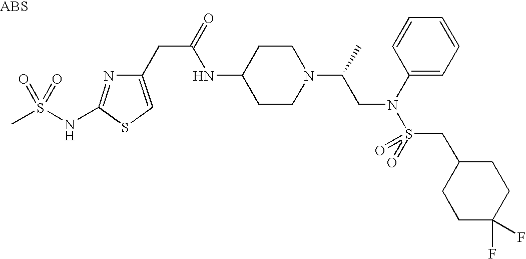 |
| 112 | 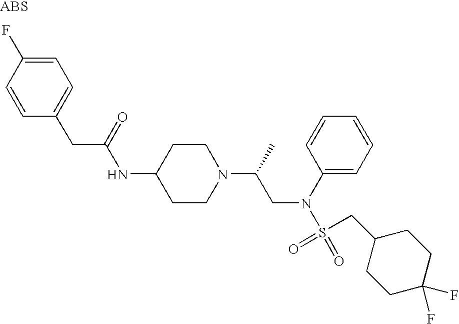 |
| 113 | 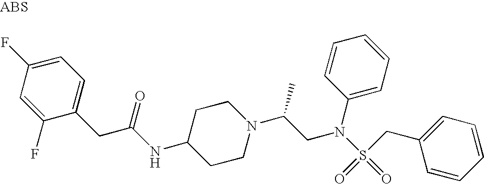 |
| 114 | 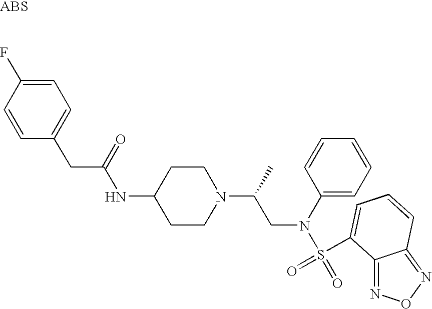 |
| 115 | 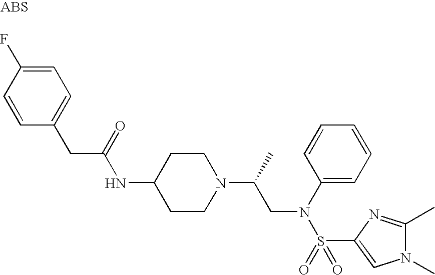 |
| 116 | 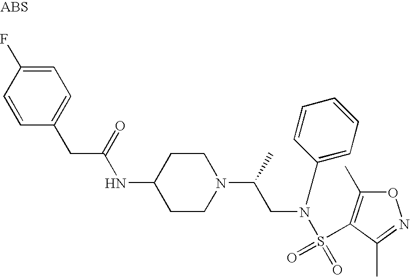 |
| 117 | 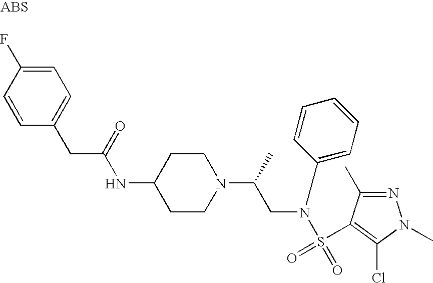 |
| 118 | 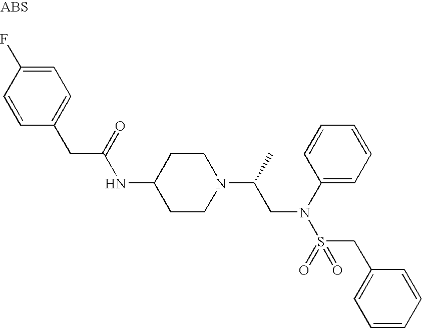 |
| 119 | 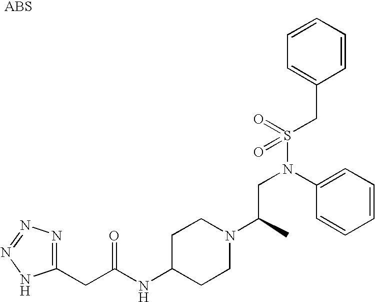 |
| 120 | 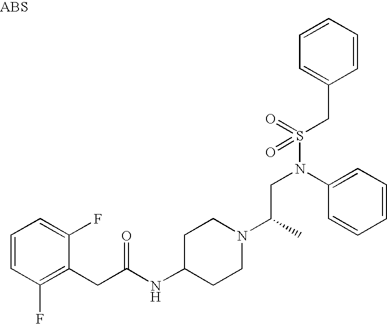 |
| 121 | 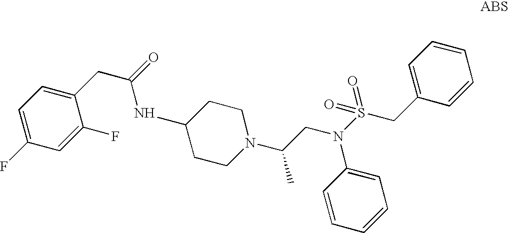 |
| 122 | 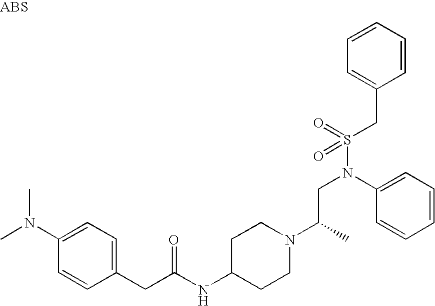 |
| 123 | 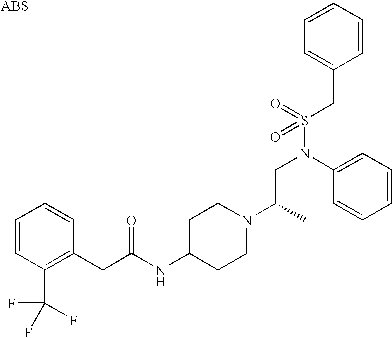 |
| 124 | 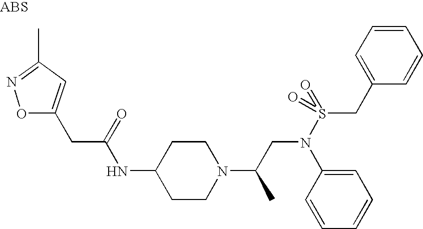 |
| 125 | 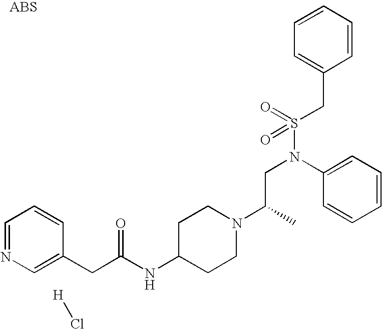 |
| 126 | 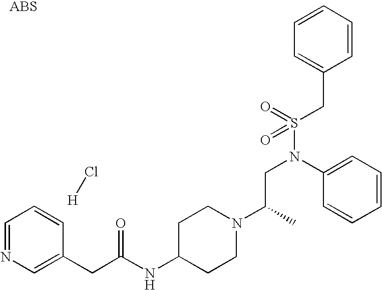 |
| 127 | 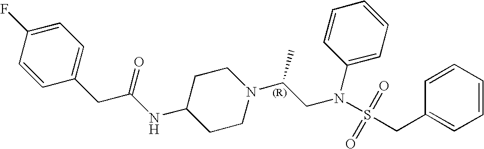 |

The invention further provides the use of piperidine derivatives of formula I and pharmaceutically acceptable salts and solvates thereof as CCR5 agonists,

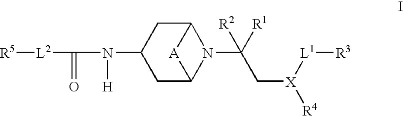

wherein
A is —CH₂—CH₂— or absent;
R¹ and R² independently are H, halo, optionally substituted alkyl, aryl, heteroaryl, cycloalkyl, cycloalkylalkyl, heterocyclyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R³ and R⁴ independently are a group selected from aryl, heteroaryl, cycloalkyl, and heterocyclyl, each group being optionally substituted by one or more substituent(s) selected from halo, cyano, HN═C(NH₂)—, SO₂R, or SO₂NR′R″ wherein R is an alkyl and R′, R″ are H or alkyl;
L¹ is CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;

### X is CR⁶ or N;

R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R⁵ is a group selected from aryl, heteroaryl, cycloalkyl, and heterocyclyl, each group being optionally substituted by one or more substituent(s) selected from halo, cyano, SO2R, or SO2NR′R″ wherein R is an alkyl and R′, R″ are H or alkyl;
R⁶ is selected from hydrogen, cyano, hydroxyl, alkoxy, carboxy, CO—NR¹⁰R¹¹, halo, C₁-C₆ alkyl and allyl;
R¹⁰ and R¹¹ independently are H, alkyl, cycloalkyl; and
L² is a single bond or C₁-C₄ alkylene, optionally substituted by one or more substituent(s) selected from halo, oxo, cyano, alkyl, hydroxyalkyl, haloalkyl, cycloalkyl, and alkoxy, or L² is CR^{a}R^{b}, wherein R^{a} and R^{b} form together with the carbon to which they are attached a carbocycle having 3 to 6 ring atoms.

### DETAILED DESCRIPTION OF THE INVENTION

As noted above, the invention relates to novel compounds of table 1, their pharmaceutically active salts and solvates and to the use of compounds of formula I as CCR5 agonists.

Preferred compounds of formula I and pharmaceutically active salts and solvates thereof are those wherein

R³, R⁴, R⁵, and L¹ are as defined above in respect of general formula I,
A is absent;
R¹ and R² independently are hydrogen, halo, or C₁-C₄ alkyl; preferably hydrogen or methyl;
X is CH, C(OH), C(CN), or N, preferably CH or N; and

### L² is CH₂.

Even more preferred compounds of formula I and pharmaceutically acceptable salts thereof are those wherein

A is absent;
R¹ is methyl;
R² is hydrogen;

### X is CH or N;

L¹, R³, R⁴ and R⁵ are defined as above in respect of general formula I; and

### L² is CH₂.

In one embodiment, preferred compounds of formula I are those of formula Ia:

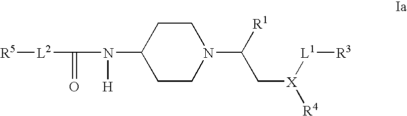

and pharmaceutically acceptable salts and solvates thereof, wherein
R¹ is hydrogen or methyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said substituents being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R³ and R⁴ independently are a group selected from aryl, heteroaryl, cycloalkyl, and heterocyclyl, each group being optionally substituted by one or more substituent(s) selected from halo, cyano, HN═C(NH₂)—, SO₂R, or SO₂NR′R″ wherein R is an alkyl and R′, R″ are H or alkyl;
X is CR⁶ or N, preferably CH or N;
R⁶ is selected from hydrogen, hydroxyl, alkoxy, carboxy, halo, C₁-C₆ alkyl, CO—NR¹⁰R¹¹, cyano, and allyl;
R¹⁰ and R¹¹ independently are H, alkyl, or cycloalkyl;
R⁵ is defined as above in respect of formula I, preferably R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, preferably R⁵ is a group selected from aryl, heteroaryl, cycloalkyl, and heterocyclyl, each group being optionally substituted by one or more substituent(s) selected from halo, cyano, SO₂R, or SO₂NR′R″ wherein R is an alkyl and R′, R″ are H or alkyl;
L¹ is CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;
L² is defined as above, preferably L² is —CR⁷R⁸—, more preferably —CH₂—; and
R⁷ and R⁸ independently are hydrogen, halo, optionally substituted alkyl, aryl, heteroaryl, cycloalkyl, or heterocyclyl, or R⁷ and R⁸, form together with the carbon atom to which they are connected a 3 to 6 membered saturated or unsaturated cycle, for example cyclopropyl, preferably R⁷ and R⁸ independently are hydrogen, methyl, ethyl, phenyl, pyridinyl, or cyclohexyl, or R⁷ and R⁸, form together with the carbon atom to which they are connected cyclopropyl, even more preferably R⁷ and R⁸ are hydrogen.

In one embodiment, preferred compounds of formula I are those of formula Ib:

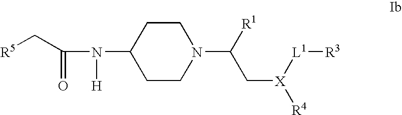

and pharmaceutically acceptable salts and solvates thereof, wherein

### X is CH or N;

R¹ is hydrogen or methyl;

L¹ is CO, CONR, CONRCH₂, CH₂CO, COCH₂CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;

R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, or sulfonyl; more preferably, R³ and R⁴ independently are a group selected from aryl, heteroaryl, cycloalkyl, and heterocyclyl, each group being optionally substituted by one or more substituent(s) selected from halo, cyano, HN═C(NH₂)—, SO₂R, or SO₂NR′R″ wherein R is an alkyl and R′, R″ are H or alkyl; and
R⁵ is defined as above in respect of formula I, preferably R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R⁵ more preferably, preferably R⁵ is a group selected from aryl, heteroaryl, cycloalkyl, and heterocyclyl, each group being optionally substituted by one or more substituent(s) selected from halo, cyano, SO2R, or SO2NR′R″ wherein R is an alkyl and R′, R″ are H or alkyl.

In one embodiment, preferred compounds of formula I are those of formula Ic:

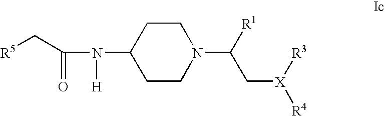

and pharmaceutically acceptable salts and solvates thereof, wherein

### X is CH or N;

R¹ is hydrogen or methyl;
R³ is defined as above in respect of formula I, preferably is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, or indolyl group may be one or more cycloalkyl, aryl, heterocyclyl or heteroaryl group; more preferably, R³ is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, each group being optionally substituted by one or more substituent(s) selected from halo, cyano, HN═C(NH₂)—, SO₂R, or SO₂NR′R″ wherein R is an alkyl and R′, R″ are H or alkyl;
R⁴ is phenyl or pyridinyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group; more preferably, R⁴ is phenyl or pyridinyl, each being optionally substituted by one or more substituent(s) selected from halo, cyano, SO2R, or SO2NR′R″ wherein R is an alkyl and R′, R″ are H or alkyl; and
R⁵ is defined as above in respect of formula (I), preferably R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl or heteroaryl group; more preferably, R⁵ is a group selected from aryl, heteroaryl, cycloalkyl, and heterocyclyl, each group being optionally substituted by halo, cyano, SO2R, or SO2NR′R″ wherein R is an alkyl and R′, R″ are H or alkyl.

In another embodiment, preferred compounds of formula I are those of formula Id:

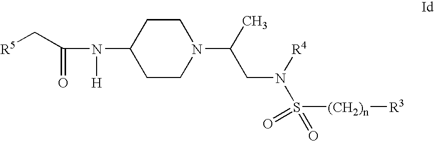

and pharmaceutically acceptable salts and solvates thereof, wherein
n is 0, 1 or 2;
R³ is aryl, heteroaryl or cycloalkyl, preferably phenyl, pyridinyl or cyclohexyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group; more preferably, R³ is phenyl, benzyl, pyridinyl, —(CH₂)-pyridinyl, cyclohexyl or —(CH₂)-cyclohexyl, each being optionally substituted by one or more substituent(s) selected from halo, cyano, SO₂R, or SO₂NR′R″ wherein R is an alkyl and R′, R″ are H or alkyl;
R⁴ is defined as above in respect of formula I, preferably is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, or indolyl group may be one or more cycloalkyl, aryl, heterocyclyl or heteroaryl group; more preferably, R⁴ is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, and most preferably R⁴ is phenyl, each group being optionally substituted by one or more substituent(s) selected from halo, cyano, HN═C(NH₂)—, SO2R, or SO2NR′R″ wherein R is alkyl and R′, R″ are H or alkyl; and
R⁵ is defined as above in respect of formula (I), preferably R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, HN═C(NH₂)—, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl or heteroaryl group; more preferably, R⁵ is a group selected from aryl, heteroaryl, cycloalkyl, and heterocyclyl, each group being optionally substituted by halo, cyano, HN═C(NH₂)—, SO₂R, or SO₂NR′R″ wherein R is an alkyl and R′, R″ are H or alkyl.

Preferably, compounds of formula Id are those of formula Id′

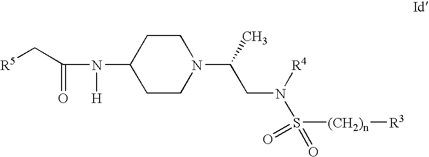

and pharmaceutically acceptable salts and solvates thereof, wherein n, R³, R⁴, and R⁵ are defined as in respect of formula Id above.

Particularly preferred compounds of formula I are those listed in Table 1 above and those of Table 2 hereafter:

TABLE 2

| Compound | Structure |
|---|---|
| 1 | 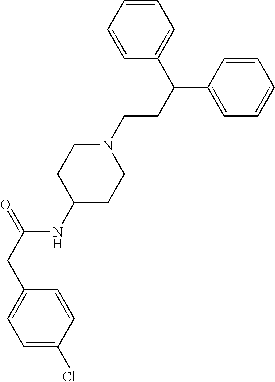 |
| 2 | 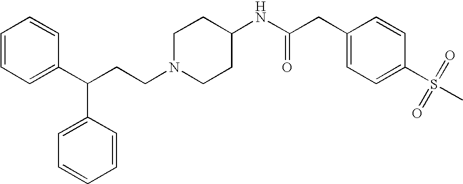 |
| 3 | 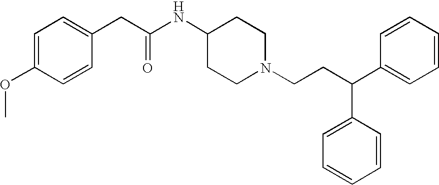 |
| 4 | 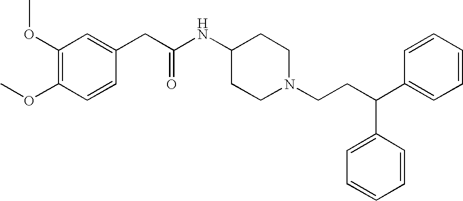 |
| 5 | 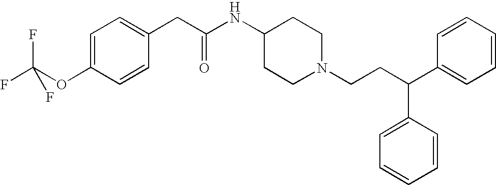 |
| 6 | 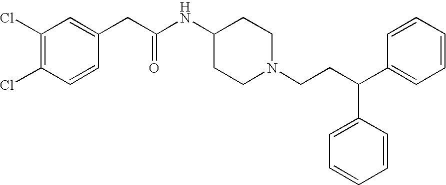 |
| 7 | 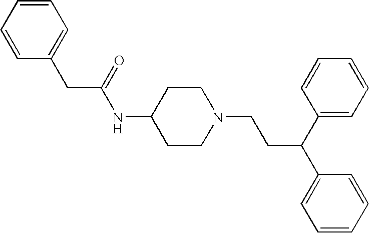 |
| 8 | 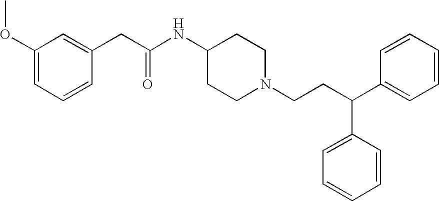 |
| 9 | 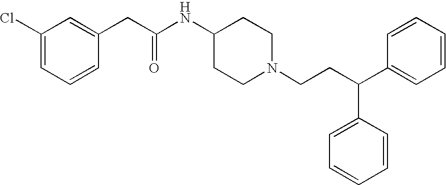 |
| 10 | 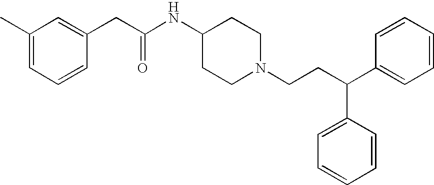 |
| 11 | 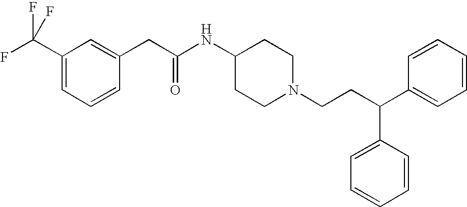 |
| 12 | 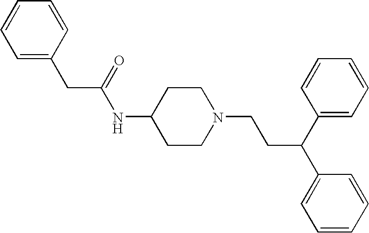 |
| 13 | 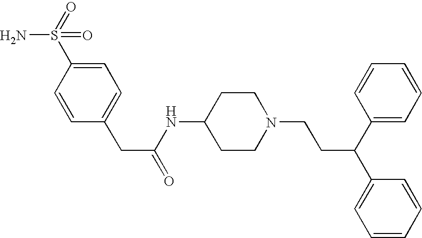 |
| 14 | 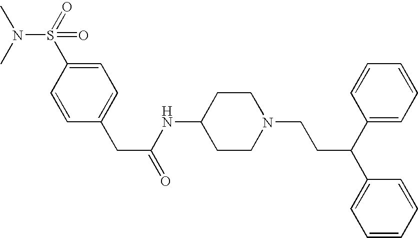 |
| 15 | 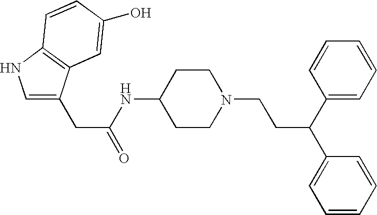 |
| 16 | 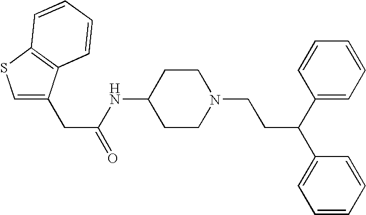 |
| 17 | 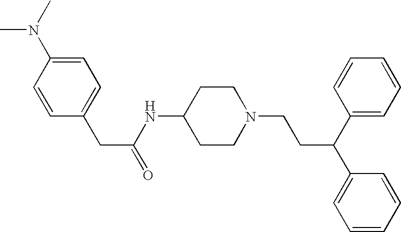 |
| 18 | 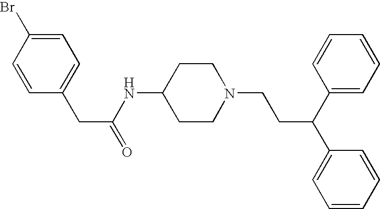 |
| 19 | 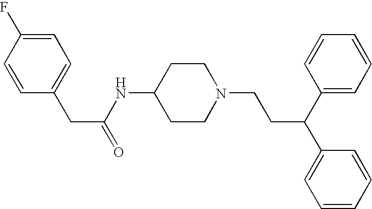 |
| 20 | 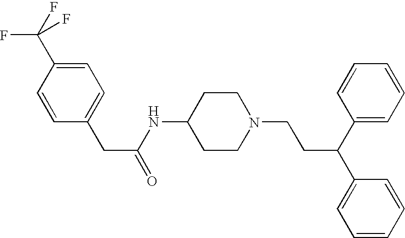 |
| 21 | 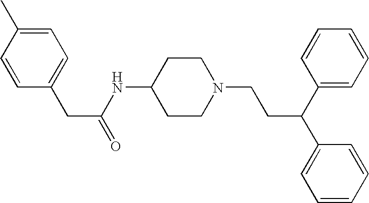 |
| 22 | 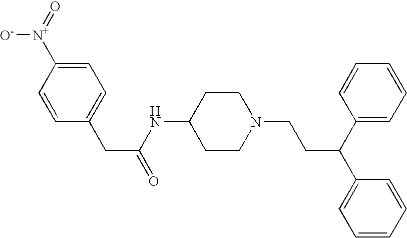 |
| 23 | 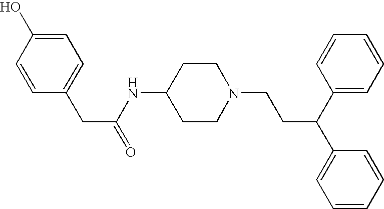 |
| 128 | 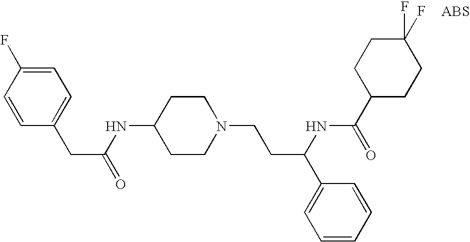 |
| 129 | 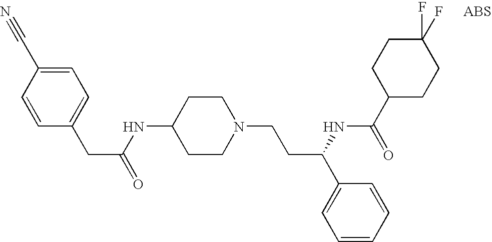 |
| 130 | 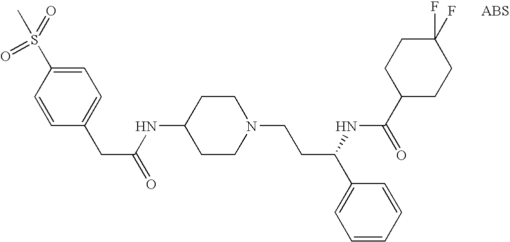 |
| 131 | 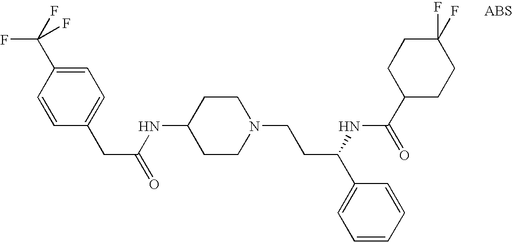 |
| 132 | 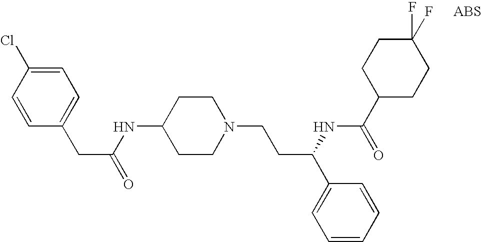 |
| 133 | 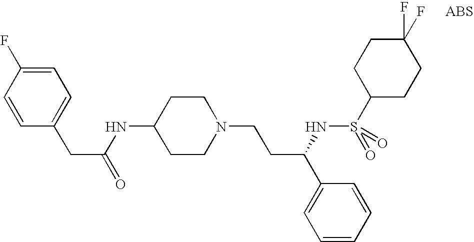 |
| 134 | 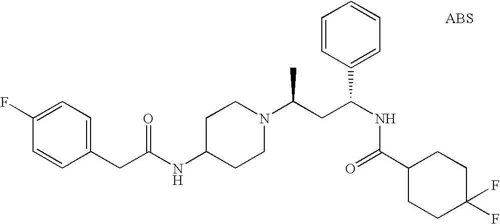 |
| 135 | 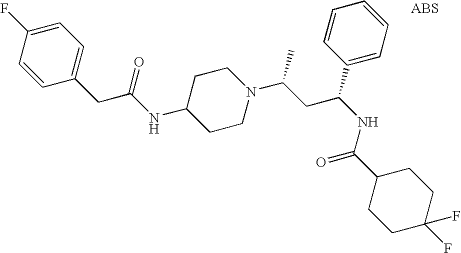 |

The compounds of formula I can be prepared by different ways with reactions known by the person skilled in the art. Reaction schemes I to III (Example section), illustrate by way of example different possible approaches.

### [Applications]

The compounds of the invention have activity as pharmaceuticals, in particular as agonists of chemokine receptor activity, especially of CC chemokine receptor activity, and even more particularly of CCR5 receptor activity. The inventors have now found, surprisingly and unexpectedly that the presence of a hydrogen atom at the amide nitrogen, which is connected to the piperidine ring in position 4, instead of an alkyl group introduces a switch from an antagonistic mode of action to an agonistic mode of action of the compounds of the invention. Without wanting to be linked by any theory, it is believed that a non hydrogen substitution of the amide nitrogen introduces specific sterical strains between the piperidine ring and the non hydrogen substituent. Due to these strains non hydrogen substituted molecules are not able to retain the bioactive conformation of the hydrogen substituted molecules and thus cannot act as agonists, but act as antagonists. It is thus believed that the agonistic activity of the compounds of the invention is due to the presence of the hydrogen atom on the amide nitrogen.

The compounds of the invention are therefore useful in the prevention or in the prevention and/or treatment of autoimmune, inflammatory, infectious, proliferative, hyperproliferative diseases, or immunologically-mediated diseases (including rejection of transplanted organs or tissues and Acquired Immunodeficiency Syndrome (AIDS)); examples of these conditions are:

(1) (the respiratory tract) obstructive diseases of airways including: chronic obstructive pulmonary disease (COPD) (such as irreversible COPD); pulmonary fibrosis; asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertonic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systematic sclerosis;

(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasiculitides erythermas, cutaneaous eosinophilias, uveitis, Alopecia greata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allorgraft rejection) acute and chronic following, for example, transplantation of kidney, heart, liver, lung bone marrow, skin or cornea; or chronic graft versus host disease; and/or

(6) (other tissues or diseases) Alzheimer's disease, multiple sclerosis, atherosclerosis, inhibiting the entry of viruses into target cells, Acquired Immunodeficiency Syndrome (AIDS), Lupus disorders (such us lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura, disorders of the menstrual cycle, glomerulonephritis or cerebral malaria, acute and chronic hepatitis B Virus (HBV) and HCV infection.

The treatment or prevention of these diseases comprises the administration of a therapeutically effective amount of a compound or pharmaceutically acceptable salt or solvate of the compounds of the invention, to a patient in need thereof. Preferably the patient is a warm-blooded animal, more preferably a human.

Preferred diseases are AIDS (HIV-1 or -2 infection), inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary disease (COPD, including pulmonary fibrosis). Additional fields of application concern certain sort of metastatic cancers and renal diseases.

In a particular preferred embodiment the disease is AIDS (HIV-1 or -2 infection).

The compounds of the present invention are also of value in inhibiting the entry of viruses (such as human immunodeficiency virus (HIV)) into target cells and, therefore, are of value in the prevention of infection by viruses (such as HIV), the treatment of infection by viruses (such as HIV) and the prevention and/or treatment of acquired immune deficiency syndrome (AIDS).

According to a further feature of the present invention there is provided a method for modulating chemokine receptor activity, especially CCR5 receptor activity, in a patient, preferably a warm blooded animal, and even more preferably a human, in need of such treatment, which comprises administering to said animal an effective amount of compound of the present invention, or a pharmaceutically acceptable salt or solvate thereof.

According to one embodiment, the compounds of the invention, their pharmaceutical acceptable salts or solvates may be administered as part of a combination therapy. Thus, are included within the scope of the present invention embodiments comprising coadministration of, and compositions and medicaments which contain, in addition to a compound of the present invention, a pharmaceutically acceptable salt or solvate thereof as active ingredient, additional therapeutic agents and/or active ingredients. Such multiple drug regimens, often referred to as combination therapy, may be used in the treatment and prevention of any of the diseases or conditions mediated by or associated with CCR5 chemokine receptor modulation, particularly infection by human immunodeficiency virus, HIV. The use of such combinations of therapeutic agents is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature.

In addition to the requirement of therapeutic efficacy, which may necessitate the use of active agents in addition to the CCR5 agonist compounds of Formula I or their pharmaceutical acceptable salts or solvates thereof, there may be additional rationales which compel or highly recommend the use of combinations of drugs involving active ingredients which represent adjunct therapy, i.e., which complement and supplement the function performed by the CCR5 chemokine receptor agonist compounds of the present invention. Such supplementary therapeutic agents used for the purpose of auxiliary treatment include drugs which, instead of directly treating or preventing a disease or condition mediated by or associated with CCR5 chemokine receptor modulation, treat diseases or conditions which directly result from or indirectly accompany the basic or underlying CCR5 chemokine receptor modulated disease or condition. For example, where the basic CCR5 chemokine receptor modulated disease or condition is HIV infection and multiplication, it may be necessary or at least desirable to treat opportunistic infections, neoplasms, and other conditions which occur as the result of the immune-compromised state of the patient being treated. Other active agents may be used with the compounds of Formula I or their pharmaceutical acceptable salts or solvates thereof, e.g., in order to provide immune stimulation or to treat pain and inflammation which accompany the initial and fundamental HIV infection.

Thus, the methods of treatment and pharmaceutical compositions of the present invention may employ the compounds of Formula I or their pharmaceutical acceptable salts or solvates thereof in the form of monotherapy, but said methods and compositions may also be used in the form of multiple therapy in which one or more compounds of Formula I or their pharmaceutically acceptable salts or solvates are coadministered in combination with one or more other therapeutic agents such as those described in detail further herein.

Preferred combinations of the present invention include simultaneous, or sequential treatments with a compound of Formula I, or a pharmaceutical acceptable salt or solvate thereof, and one or more inhibitors of HIV protease and/or inhibitors of HIV reverse transcriptase, preferably selected from the class of non-nucleoside reverse transcriptase inhibitors (NNRTI), including but not limited to nevirapine, delavirdine and efavirenz; from among the nucleoside/nucleotide inhibitors, including but not limited to zidovudine, didanosine, zalcitabine, stavudine, lamivudine, abacavir, adefovir and dipivoxil; and from among the protease inhibitors, including but not limited to indinavir, ritonavir, saquinavir, nelfinavir, lopinavir, and amprenavir.

Other agents useful in the above-described preferred embodiment combinations of the present invention include current and to-be-discoveredinvestigational drugs from any of the above classes of inhibitors, including but not limited to FTC, PMPA, fozivudinetidoxil, talviraline, S-1153, MKC-442, MSC-204, MSH-372, DMP450, PNU-140690, ABT-378, KNI-764, TMC120 and TMC125.

There is also included within the scope of the preferred embodiments of the present invention, combinations of a compound of Formula I, or a pharmaceutical acceptable salt or solvate thereof, together with a supplementary therapeutic agent used for the purpose of auxiliary treatment, wherein said supplementary therapeutic agent comprises one or more members independently selected from the group consisting of proliferation inhibitors, e.g., hydroxyurea; immunomodulators, e.g., sargramostim, and various forms of interferon or interferon derivatives; fusion inhibitors, e.g., AMD3100, T-20, T-1249, PRO-140, PRO-542, AD-349, BB-10010 and other chemokine receptor agonists/antagonists; tachykinin receptor modulators, e.g. NK1 antagonists; integrase inhibitors, e.g., AR177; RNaseH inhibitors; inhibitors of viral transcription and RNA replication; and other agents that inhibit viral infection or improve the condition or outcome of HIV-infected individuals through different mechanisms.

Preferred methods of treatment of the present invention for the prevention of HIV infection, or treatment of aviremic and asymptomatic subjects potentially or effectively infected with HIV, include but are not limited to administration of a member independently selected from the group consisting of: (i) a compound within the scope of Formula I or a pharmaceutical acceptable salt or solvate thereof as disclosed herein; (ii) one NNRTI in addition to a compound of (i); (iii) two NRTI in addition to a compound of (i); (iv) one NRTI in addition to the combination of (ii); and (v) a compound selected from the class of protease inhibitors used in place of a NRTI in combinations (iii) and (iv).

The preferred methods of the present invention for therapy of HIV-infected individuals with detectable viremia or abnormally low CD4 counts further include as a member to be selected: (vi) treatment according to (i) above in addition to the standard recommended initial regimens for the therapy of established HIV infections. Such standard regimens include but are not limited to an agent from the class of protease inhibitors in combination with two NRTIs; and (vii) a standard recommended initial regimens for the therapy of established HIV infections, where either the protease inhibitor component, or one or both of the NRTIs is/are replaced by a compound within the scope of Formula I as disclosed herein.

The preferred methods of the present invention for therapy of HIV-infected individuals that have failed antiviral therapy further include as a member to be selected: (viii) treatment according to (i) above, in addition to the standard recommended regimens for the therapy of such patients; and (ix) a standard recommended initial regimens for the therapy of patients who have failed antiretroviral therapy, where either one of the protease inhibitor components, or one or both of the NRTIs is/are replaced by a compound within the scope of Formula I or a pharmaceutical acceptable salt or solvate thereof as disclosed herein.

Additional combinations for use according to the invention include combination of a compound of Formula I, or a pharmaceutical acceptable salt or solvate thereof with another CCR5 modulator, such as a CCR5 agonist; a CCR5 antagonist, such as N-{(1S)-3-[3-(3-isopropyl-5-methyl-4H-1,2,4-triazol-4-yl)-exo-8-azabicyclo[3.2.1]oct-8-yl]-1-phenylpropyl}-4,4-difluorocyclohexanecarboxamide; a CCR1 antagonist, such as BX-471; a beta adrenoceptor agonist, such as salmeterol; a corticosteroid agonist, such fluticasone propionate; a LTD4 antagonist, such asmontelukast; a muscarinic antagonist, such as tiotropium bromide; a PDE4 inhibitor, such ascilomilast or roflumilast; a COX-2 inhibitor, such ascelecoxib, valdecoxib or rofecoxib; an alpha-2-delta ligand, such as gabapentin or pregabalin; a beta-interferon, such as REBIF; a TNF receptor modulator, such as aTNF-alpha inhibitor (e.g. adalimumab); a HMG CoA reductase inhibitor, such as a statin (e.g. atorvastatin); or an immunosuppressant, such as cyclosporine; or a macrolide such as tacrolimus.

In the above-described preferred embodiment combinations of the present invention, the compound of formula I, a pharmaceutically acceptable salt or solvate thereof and other therapeutic active agents may be administered in terms of dosage forms either separately or in conjunction with each other, and in terms of their time of administration, either serially or simultaneously. Thus, the administration of one component agent may be prior to, concurrent with, or subsequent to the administration of the other component agent(s).

The invention also provides pharmaceutical compositions comprising a compound of formula I or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as active ingredient, additional therapeutic agents and/or active ingredients.

Another object of this invention is a medicament comprising at least one compound of the invention, or a pharmaceutically acceptable salt or solvate thereof, as active ingredient.

The invention also provides the use of a compound of formula I or a pharmaceutically acceptable salt or solvate thereof for the manufacture of a medicament. Preferably, the medicament is used for the treatment or prevention of autoimmune, inflammatory, infectious, proliferative or hyperprolifeartive diseases, or immunologiaccly mediated diseases (including rejection of transplanted organs or tissues and Acquired Immunodeficiency Syndrome (AIDS)); examples of these conditions are:

(1) (the respiratory tract) obstructive diseases of airways including: chronic obstructive pulmonary disease (COPD) (such as irreversible COPD); pulmonary fibrosis; asthma (such as bronchial, allergic, intrinsic, extrinsic or dust asthma, particularly chronic or inveterate asthma (for example late asthma or airways hyper-responsiveness)); bronchitis (such as eosinophilic bronchitis); acute, allergic, atrophic rhinitis or chronic rhinitis including rhinitis caseosa, hypertonic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systematic sclerosis;

(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasiculitides erythermas, cutaneaous eosinophilias, uveitis, Alopecia greata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung bone marrow, skin or cornea; or chronic graft versus host disease; and/or

(6) (other tissues or diseases) Alzheimer's disease, multiple sclerosis, atherosclerosis, inhibiting the entry of viruses into target cells, Acquired Immunodeficiency Symdrome (AIDS), Lupus disorders (such us lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura, disorders of the menstrual cycle, glomerulonephritis or cerebral malaria, acute and chronic hepatitis B Virus (HBV) and HCV infection.

Preferred diseases are AIDS (HIV-1 or -2 infection), inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary disease (COPD, including pulmonary fibrosis). Additional fields of application concern certain sort of metastatic cancers and renal diseases.

In a particular preferred embodiment the disease is AIDS (HIV-1 or -2 infection).

The invention also provides the use of a compound of formula I or a pharmaceutically acceptable salt or solvate thereof for the manufacture of a medicament for inhibiting the entry of viruses (such as human immunodeficiency virus (HIV)) into target cells and, therefore, for the prevention of infection by viruses (such as HIV), the treatment of infection by viruses (such as HIV) and the prevention and/or treatment of acquired immune deficiency syndrome (AIDS).

According to a further feature of the present invention there is provided the use of a compound of formula I or a pharmaceutically acceptable salt or solvate thereof for the manufacture of a medicament for modulating chemokine receptor activity, especially CCR5 receptor activity, in a patient, in need of such treatment, which comprises administering to said patient an effective amount of compound of the present invention, or a pharmaceutically acceptable salt or solvate thereof.

Preferably, the patient is a warm-blooded animal, more preferably a human.

As set forth above, the compounds of the invention, their pharmaceutically acceptable salts or solvates may be used in monotherapy or in combination therapy, such as bi- or tritherapy. Thus, according to one embodiment, the invention provides the use of a compound of the invention for the manufacture of a medicament for at least one of the purposes described above, wherein said medicament is administered to a patient in need thereof, preferably a warm-blooded animal, and even more preferably a human, in combination with at least one additional therapeutic agent and/or active ingredient. The benefits and advantages of such a multiple drug regimen, possible administration regimens as well as suitable additional therapeutic agents and/or active ingredients are those described above.

Generally, for pharmaceutical use, the compounds of the inventions may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.05 and 1000 mg, and usually between 1 and 500 mg, of the at least one compound of the invention, e.g. about 10, 25, 50, 100, 200, 300 or 400 mg per unit dosage.

Usually, depending on the condition to be prevented or treated and the route of administration, the active compound of the invention will usually be administered between 0.01 to 100 mg per kilogram, more often between 0.1 and 50 mg, such as between 1 and 25 mg, for example about 0.5, 1, 5, 10, 15, 20 or 25 mg, per kilogram body weight day of the patient per day, which may be administered as a single daily dose, divided over one or more daily doses, or essentially continuously, e.g. using a drip infusion.

### DEFINITIONS

The definitions and explanations below are for the terms as used throughout the entire application, including both the specification and the claims.

When describing the compounds of the invention, the terms used are to be construed in accordance with the following definitions, unless indicated otherwise.

Where groups may be substituted, such groups may be substituted with one or more substituents, and preferably with one, two or three substituents. Substituents may be selected from but not limited to, for example, the group comprising halogen, hydroxyl, oxo, nitro, amido, carboxy, amino, cyano haloalkoxy, and haloalkyl.

As used herein the terms such as “alkyl, aryl, or cycloalkyl, each being optionally substituted with . . . ” or “alkyl, aryl, or cycloalkyl, optionally substituted with . . . ” encompasses “alkyl optionally substituted with . . . ”, “aryl optionally substituted with . . . ” and “cycloalkyl optionally substituted with . . . ”.

The term “halo” or “halogen” means fluoro, chloro, bromo, or iodo.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein.

Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

The term “hydroxyalkyl” includes but is not limited to hydroxymethyl, 1-hydroxyethyl, 2-hydroxyethyl, 1-hydroxypropyl, 2-hydroxypropyl, 2-hydroxy-2-methylethyl, 1-hydroxypropyl, 2-hydroxypropyl, 3-hydroxypropyl, 1-hydroxybutyl, 2-hydroxybutyl, 3-hydroxybutyl, 4-hydroxybutyl, 2-hydroxy-2-methylpropyl, 1-(hydroxymethyl)-2-methylpropyl, 1,1-dimethyl-2-hydroxyethyl, 5-hydroxypentyl, 2-methyl-3-hydroxypropyl, 3,4-dihydroxybutyl, and so forth “Alkoxyalkyl” refers to an alkyl group substituted with one to two R^{b}, wherein R^{b} is alkoxy as defined below. For example heterocyclylalkyl refers to an alkyl group substituted with one to two R^{f}, wherein R^{f} is heterocyclyl as defined below. For example, “aralkyl” or “arylalkyl” refers to a substituted alkyl group as defined above wherein at least one of the alkyl substituents is an aryl as defined below, such as benzyl. For example, “heteroarylalkyl” refers to a substituted alkyl group as defined above, wherein at least one of the alkyl substituents is a heteroaryl as defined below, such as pyridinyl.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl, with cyclopropyl being particularly preferred. An “optionally substituted cycloalkyl” refers to a cycloalkyl having optionally one or more substituent(s) (for example 1 to 3 substituent(s), for example 1, 2 or 3 substituent(s)), selected from those defined above for substituted alkyl. When the suffix “ene” is used in conjunction with a cyclic group, this is intended to mean the cyclic group as defined herein having two single bonds as points of attachment to other groups.

The term “cycloalkylalkyl” includes but is not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl and the like.

The term “alkenyl” as used herein refers to an unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like. The term “alkynyl” as used herein refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Non limiting examples of alkynyl groups are ethynyl, 2-propynyl, 2-butynyl, 3-butynyl, 2-pentynyl and its isomers, 2-hexynyl and its isomers—and the like. The term “alkylene” includes methylene, ethylene, methylmethylene, propylene, ethylethylene, and 1,2-dimethylethylene. “Cycloalkylene” herein refers to a saturated homocyclic hydrocarbyl biradical of Formula CₙH₂ₙ₋₂. Cycloalkylene groups of this invention preferably comprise the same number of carbon atoms as their cycloalkyl radical counterparts.

Suitable cycloalkylene groups are C₃₋₆ cycloalkylene group, preferably a C₃₋₅ cycloalkylene (i.e. 1,3-cyclopropylene, 1,1-cyclopropylene, 1,1-cyclobutylene, 1,2-cyclobutylene, 1,3-cyclopentylene, or 1,1-cyclopentylene), more preferably a C₃₋₄ cycloalkylene (i.e. 1,3-cyclopropylene, 1,1-cyclopropylene, 1,1-cyclobutylene, 1,2-cyclobutylene). The terms “heterocyclyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl; piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “aryl” as used herein refers to a polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, naphthalen-1- or -2-yl, 4-, 5-, 6 or 7-indenyl, 1-2-, 3-, 4- or 5-acenaphtylenyl, 3-, 4- or 5-acenaphtenyl, 1-, 2-, 3-, 4- or 10-phenanthryl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

The term “arylene” as used herein is intended to include divalent carbocyclic aromatic ring systems such as phenylene, biphenylylene, naphthylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like.

Where at least one carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

The bonds from an asymmetric carbon in compounds of the present invention may be depicted herein using a solid line [symbol] a zigzag line [symbol] a solid wedge [symbol] or a dotted wedge ( [symbol] ). The use of either a solid or dotted wedge to depict bonds from an asymmetric carbon atoms is meant to indicate that only the stereoisomer shown is meant to be included.

The compounds of the invention may also contain more than one asymmetric carbon atome. In those compounds, the use of a solid line to depict bonds from asymmetric carbon atoms is meant to indicate that all possible stereoisomers are meant to be included, unless it is clear from the context that a specific stereoisomer is intended.

The compounds of the invention may be in the form of pharmaceutically acceptable salts. Pharmaceutically acceptable salts of the compounds of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula I may be prepared by one or more of these methods:

(i) by reacting the compound of Formula I with the desired acid;

(ii) by reacting the compound of Formula I with the desired base;

(iii) by removing an acid- or base-labile protecting group from a suitable precursor of the compound of Formula I or by ring-opening a suitable cyclic precursor, for example, a lactone or lactam, using the desired acid; or

(iv) by converting one salt of the compound of Formula I to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The term ‘solvate’ is used herein to describe a molecular complex comprising the compound of the invention and one or more pharmaceutically acceptable solvent molecules, for example, ethanol. The term ‘hydrate’ is employed when said solvent is water.

All references to compounds of formula I include references to salts, solvates, multi-component complexes and liquid crystals thereof and to solvates, multi-component complexes and liquid crystals of salts thereof.

The compounds of the invention include compounds of formula I as hereinbefore defined, including all polymorphs and crystal habits thereof, prodrugs and isomers thereof (including optical, geometric and tautomeric isomers) and isotopically-labeled compounds of formula I.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I above.

The invention also generally covers all pharmaceutically acceptable predrugs and prodrugs of the compounds of Formula I.

The term “pro-drug” as used herein means the pharmacologically acceptable derivatives such as esters, amides and phosphates, such that the resulting in vivo biotransformation product of the derivative is the active drug. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

The term “Patient” refers to a warm-blooded animal, more preferably a human, who/which is awaiting or receiving medical care or is or will be the object of a medical procedure.

The term “human” refers to suject of both genders and at any stage of development (i.e. neonate, infant, juvenile, adolescent, adult).

The term “transplant” refers to the grafting, implantation or transplantation of organs, tissues, cells (e.g., bone marrow) and/or biocompatible materials onto or into the body of an animal. The term encompasses the transfer of tissues from one part of the animal's body to another part and the transfer of organs, tissues, and/or cells obtained from a donor animal (either directly or indirectly such as an organ or tissue produced in vitro by culturing cells obtained from the animal) into a recipient animal. The animal is suitably a warm-blooded vertebrate, is typically a mammal, and is especially a primate (e.g., a human). The term “transplant rejection” means any immune reaction in the recipient directed against grafted organs, tissues, cells, and/or biocompatible materials.

The term “therapeutically effective amount” (or more simply an “effective amount”) as used herein means the amount of active agent or active ingredient (e.g., chemokine receptor agonist, especially a CCR5 receptor agonist) which is sufficient to achieve the desired therapeutic or prophylactic effect in the individual to which it is administered.

The term “administration”, or a variant thereof (e.g., “administering”), means providing the active agent or active ingredient (e.g., a CCR5 agonist), alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the recipient thereof.

The term “agonist” as used herein means a ligand that activates an intracellular response when it binds to a receptor. An agonist according to the invention may promote internalization of a cell surface receptor such that the cell surface concentration of a receptor is decreased or remove.

The term “antagonist” as used herein means a ligand which competitively binds to a receptor at the same site as an agonist, but does not activate an intracellular response initiated by an active form of the receptor. An antagonist thereby inhibits the intracellular response induced by an agonist.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, liposomes.

The present invention will be better understood with reference to the following examples. These examples intended to representative of specific embodiments of the invention, and are not intended as limiting the scope of the invention.

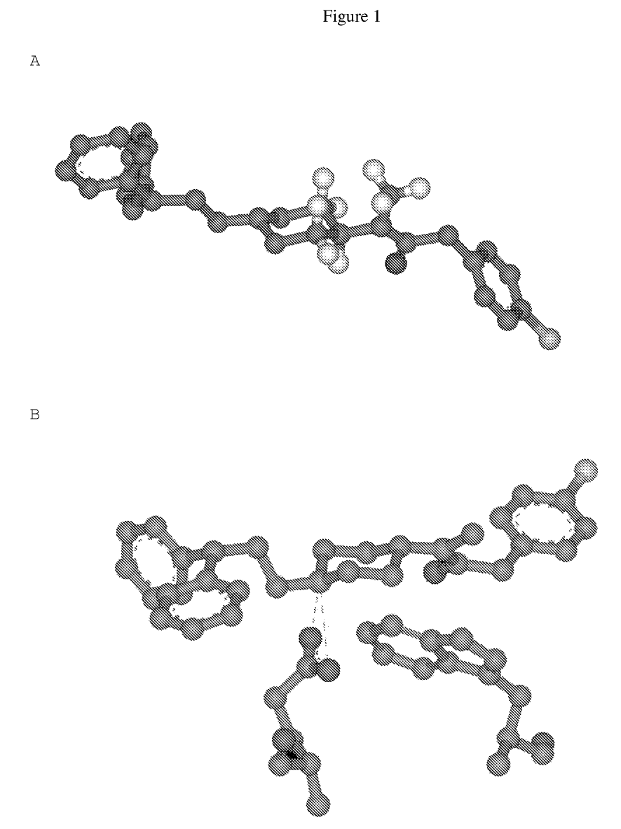

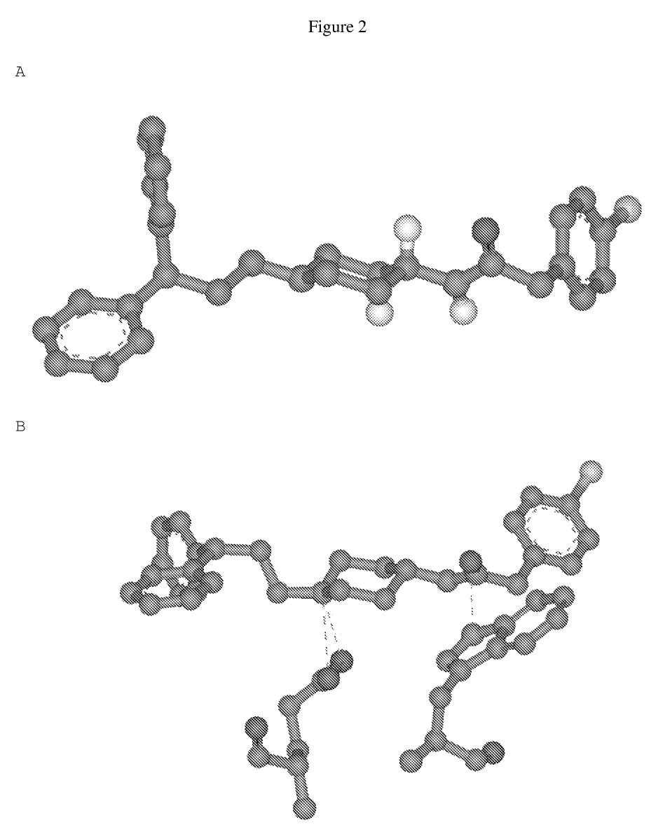

FIGS. 1 and 2 represent the bioactive conformation of the products:

FIG. 1: (A) Bioactive conformation of the antagonist compound no 61 retrieved from Molecular Modeling studies, only displaying important hydrogens; (B) Bioactive conformation of the antagonist compound no 61 in the binding pocket of the CCR5 receptor. Aminoacids E7.39 and W6.48 are displayed. Hydrogen bonds are shown in dashed lines (Hydrogens are not shown for sake of clarity).

FIG. 2: (A) Bioactive conformation of the agonist compound no 1 retrieved from Molecular Modeling studies, only displaying important hydrogens; (B) Bioactive conformation of the agonist compound no 1 in the binding pocket of the CCR5 receptor. Aminoacids E7.39 and W6.48 are displayed. Hydrogen bonds are shown in dashed lines (Hydrogens are not shown for sake of clarity).

### CHEMISTRY EXAMPLES

All temperatures are expressed in ° C. and all reactions were carried out at room temperature unless otherwise stated.

Analytical thin layer chromatography (TLC) was used to monitor reactions, establish flash chromatography conditions and verify purity of intermediates or final products. TLC plates used were Merck TLC aluminium sheet silica gel 60 F₂₅₄ purchased from VWR International. TLC plates were revealed using ultraviolet irradiation (wavelength=254 nm) at room temperature or bromocresol green spray reagent at 0.1% in propan-2-ol purchased from VWR International upon heating at 160° C. or KMnO₄ revelator upon heating at 160° C. The KMnO₄ revelator was prepared by dissolving 3 g of potassium permanganate, 20 g of sodium carbonate, 0.5 g of sodium hydroxide in 100 mL of distilled water.

HPLC-MS spectra were obtained on Waters instruments using Electropsray ionization (ESI). Samples are injected by a Waters 2767 sample manager. A Waters 2525 binary pump module is linked to a Waters 2996 photodiode array detector and a Waters micromass ZQ-2000. The column used is a Sunfire C18 5μ; 4.6*50 mm. Eluent is a mixture of solution A (0.1% TFA in H₂O) and solution B (0.1% TFA in ACN): 5% solution B for 1 min, gradient from 5% solution B to 95% solution B over 4 min, 95% solution B for 0.2 min and 5% solution B for 0.8 min. Solvents, reagents and starting materials were purchased from well known chemical suppliers such as for example Sigma Aldrich, Acros Organics, Eurisotop, VWR International, Sopachem and Polymer labs.

### Abbreviations

### ACN: Acetonitrile,

### DCM: Dichloromethane,

### DCE: Dichloroethane,

### DIEA: N,N-Diisopropylethylamine,

### DMF: N,N-dimethylformamide,

Et₂O: Diethyl ether,
EtOAc: Ethyl acetate,

### EtOH: Ethanol,

HOBt: 1-hydroxybenzotriazole,
LiAlH₄: Lithium aluminium hydride,

### MeOH: Methanol,

NaBH(OAc)₃: sodium triacetoxyborohydride,
RT: Room temperature,

### TEA: Triethylamine,

TBTU: O-(1H-Benzotriazol-1-yl)-N,N,N′,N′-tetramethyluronium tetrafluoroborate,

### Y: Yield,

g: grams,
mg: milligrams,
L: liters,
mL: milliliters,
μL: microliters,
mol: moles,
mmol: millimoles,
h: hours,
min: minutes,
TLC: thin layer chromatography,
MW: molecular weight,
eq: equivalent,
μwave: microwave,
THF: tetrahydrofuran,
Ac: acetyl,
T_{R}: retention time.

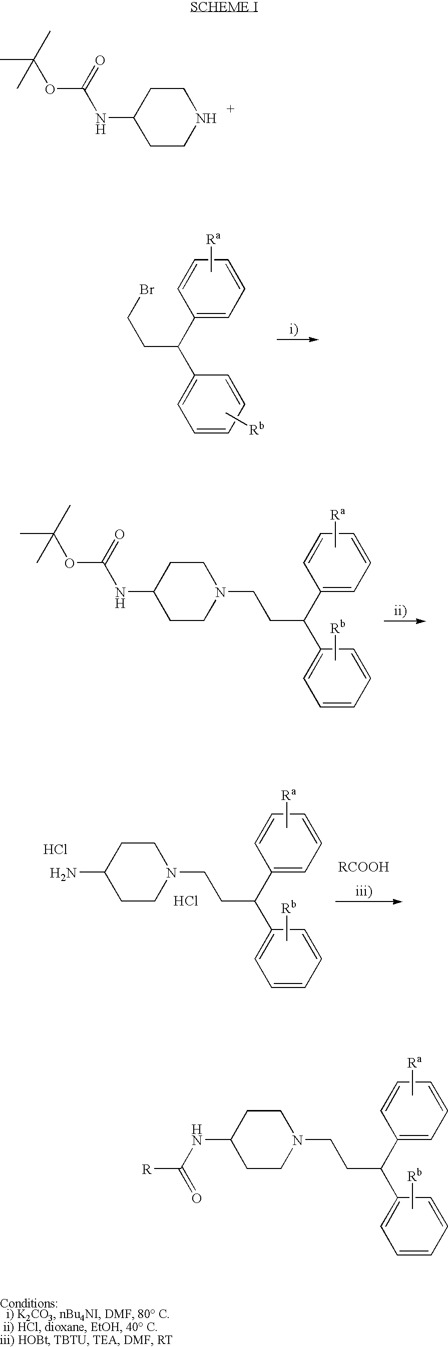

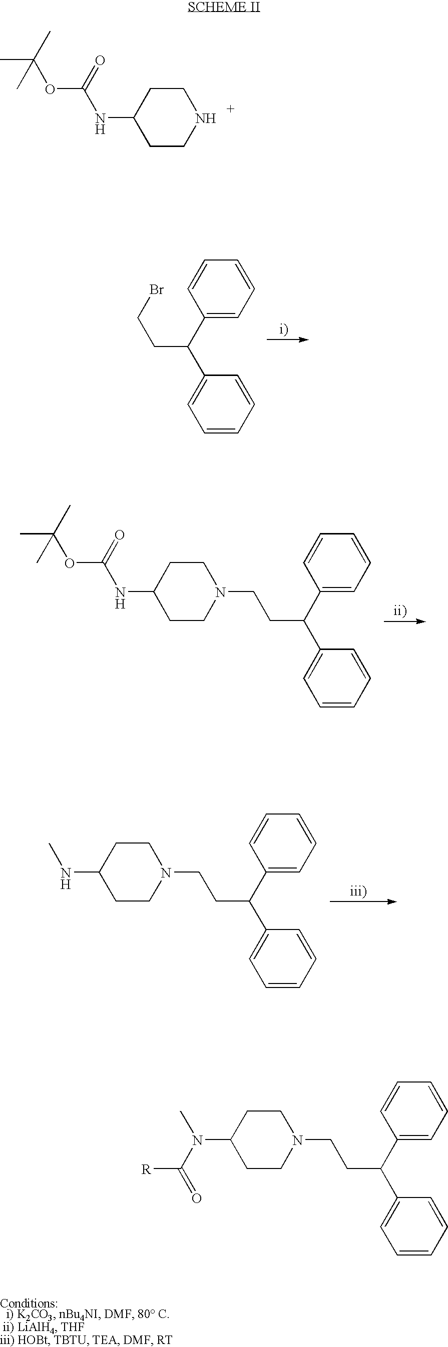

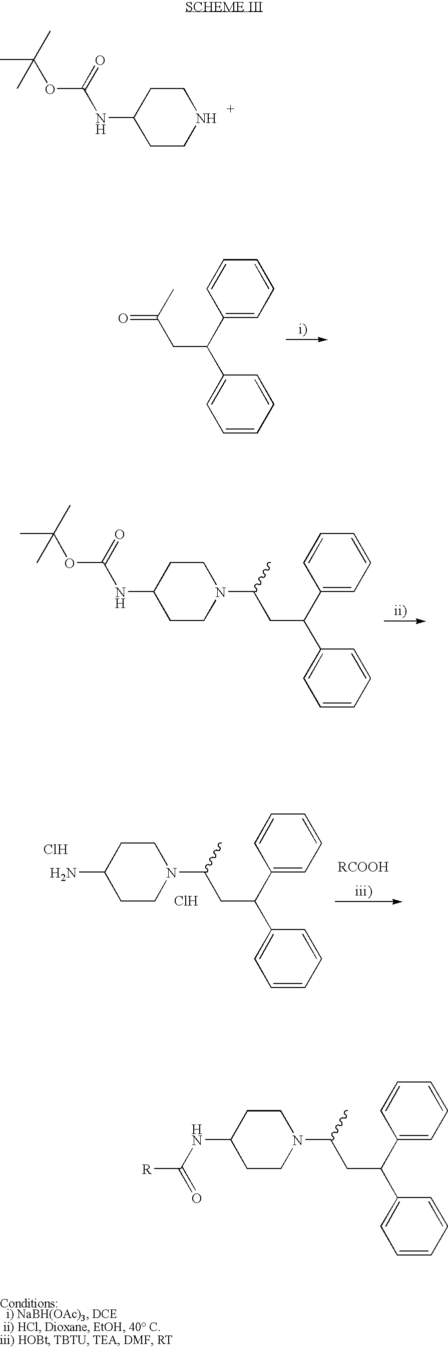

### Synthesis of Intermediate 1: 1-(3,3-diphenyl-propyl)-4-N-tertbutoxycarbonylamino-piperidine

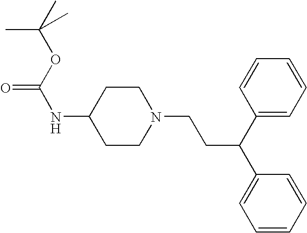

To a solution of N-tertbutoxycarbonyl-4-aminopiperidine (1.50 g; 7.50 mmol) in ACN (15 ml) were added 3,3-diphenylpropyl bromide (2.27 g; 8.25 mmol), tetrabutylammonium iodide (277 mg; 0.75 mmol) and potassium carbonate (2.07 g; 15 mmol). The mixture was refluxed overnight. The reaction mixture was cooled and filtered over silica gel and concentrated. The residue was purified by silica gel chromatography (eluent: DCM and EtOAc) to afford after dry evaporation under vacuum the title intermediate as a light yellow oil (2.63 g; Y: 90

MS: (M+H)⁺=395 T_{R}=2.86 min.

### Synthesis of Intermediate 2: 1-(3,3-diphenyl-propyl)-4-amino-piperidine dihydrochloride salt

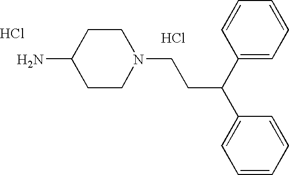

To a solution of 1-(3,3-diphenyl-propyl)-4-N-tertbutoxycarbonylamino-piperidine (2.63 g, 7.7 mmol) in ethanol (17 mL), was added hydrogen chloride in dioxane (4M solution) (25 mL, 99.9 mmol, 15 eq). The reaction mixture was stirred for 2.5 hours at 40° C. and then concentrated under vacuum to afford the title compound as a light orange intermediate (2.5 g; Y: 88%).

MS: (M+H)⁺=295, T_{R}=2.18 min.

### Synthesis of Intermediate 3: 1-(3,3-diphenyl-propyl)-4-N-methyl-amino-piperidine

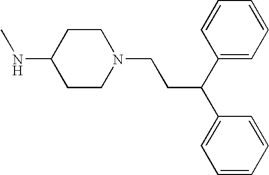

To a solution of 1-(3,3-diphenyl-propyl)-4-N-tertbutoxycarbonylamino-piperidine (455 mg; 1.15 mmol) in THF (5 ml) at 0° C. was carefully added a solution of LiAlH₄ in THF (3 mL; 2.9 mmol). The reaction mixture was stirred 1 hour at 0° C., 2 hours at RT, and then stirred overnight at 60° C. The mixture was then cooled at 0° C., quenched with a aqueous NaOH solution (1 molar) and extracted with EtOAc. The organic phase was washed with water, dried over MgSO₄ and concentrated under reduced pressure. The residue was purified by silica gel chromatography (eluent: DCM/MeOH/Et₃N) to afford after dry evaporation under vacuum the title intermediate (170 mg)

MS: (M+H)⁺=309 T_{R}=2.14 min.

### Synthesis of Intermediate 4: 1-(1-methyl-3,3-diphenyl-propyl)-4-N-tertbutoxycarbonylamino-piperidine

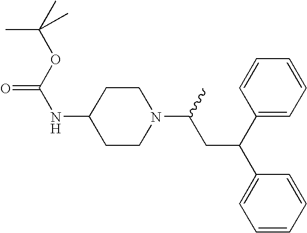

To a solution of N-tertbutoxycarbonyl-4-aminopiperidine (156 mg; 0.78 mmol), 4,4-diphenylbutan-2-one (175 mg; 0.78 mmol) in DCE (7 ml) was added sodium triacetoxyborohydride (331 mg; 1.56 mmol). The reaction mixture was stirred overnight at 85° C. DCM was added to the reaction mixture that was washed with a saturated aqueous solution of NaHCO₃ and water. The organic layer was dried over MgSO₄ and concentrated under reduced pressure. The residue was purified by silica gel chromatography (eluent: DCM/MeOH) to afford after dry evaporation under vacuum the title intermediate as an oil (51.3 mg; Y: 16%).

MS: (M+H)⁺=409 T_{R}=2.93 min.

### Synthesis of Intermediate 5: 1-(1-methyl-3,3-diphenyl-propyl)-4-amino-piperidine dihydrochloride salt

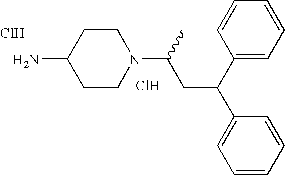

To a solution of 1-(1-methyl-3,3-diphenyl-propyl)-4-N-tertbutoxycarbonylamino-piperidine (50 mg; 0.13 mmol) in ethanol (0.5 mL), was added hydrogen chloride in dioxane (4M solution) (492 μL, 1.97 mmol, 15 eq). The reaction mixture was stirred for 2.5 hours at 40° C. and then concentrated under vacuum to afford the title intermediate as an oil (33 mg; Y: 66%).

MS: (M+H)⁺=309, T_{R}=2.2 min.

### Synthesis of Intermediate 6: (1-methanesulfonyl-piperidin-4-yl)-acetic acid ethyl ester

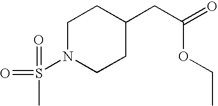

To a solution of 2-(piperidin-4-yl)-acetic acid ethyl ester (3 mmol, 1 eq) and TEA (742 μL, 3.9 mmol, 1.3 eq) in DCM at 0° C., was added dropwise methanesulfonyl chloride (302 μL, 3.9 mmol, 1.3 eq). The reaction mixture was stirred overnight at RT and then washed with an aqueous solution of NaHCO₃ and water. The organic layer was then concentrated under vacuum to afford the title intermediate.

### Synthesis of Intermediate 7: (1-methanesulfonyl-piperidin-4-yl)-acetic acid

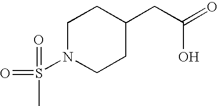

To a solution of (1-methanesulfonyl-piperidin-4-yl)-acetic acid ethyl ester (3 mmol) in a THF/H₂O (1/1) mixture was added lithium hydroxide (6 eq). The reaction mixture was stirred overnight at RT. Water was added and the mixture was extracted with DCM. The aqueous phase was collected, acidified with an aqueous solution of HCl (1M), and extracted with EtOAc. The organic layer was dried with MgSO₄ and then concentrated under vacuum to afford the title intermediate.

### Synthesis of Intermediate 8: (4-methanesulfonyl-piperazin-1-yl)-acetic acid ethyl ester

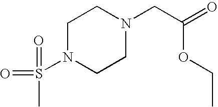

To a solution of 1-(ethoxycarbonylmethyl)piperazine (˜3 mmol, 1 eq) and TEA (742 μL, 3.9 mmol, 1.3 eq) in DCM at 0° C., was added dropwise methanesulfonyl chloride (302 μL, 3.9 mmol, 1.3 eq). The reaction mixture was stirred overnight at RT and then washed with an aqueous solution of NaHCO₃ and water. The organic layer was then concentrated under vacuum to afford the title intermediate.

### Synthesis of Intermediate 9: (4-methanesulfonyl-piperazin-1-yl)-acetic acid

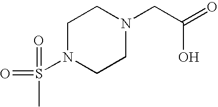

To a solution of (4-methanesulfonyl-piperazin-1-yl)-acetic acid ethyl ester (3 mmol) in a THF/H₂O (1/1) mixture was added Lithium hydroxide (6 eq). The reaction mixture was stirred overnight at RT. Water was added and the mixture was extracted with DCM. The aqueous phase was collected, acidified with HCl (1M), and extracted with EtOAc. The organic layer was dried with MgSO₄ and then concentrated under vacuum to afford the title intermediate.

### Synthesis of Intermediate 10: 4-chlorosulfonylacetic acid

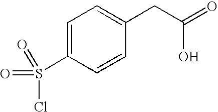

Clorosulfonic acid (391 μL, 5.88 mmol) was heated to 40° C. and phenylacetic acid (200 mg, 1.47 mmol) was added slowly. The reaction mixture was stirred for 1.5 hours at 40° C. then cooled and ice was carefully added to the mixture. The filtrate was by filtration and dried under vacuum to afford the title intermediate. (230 mg; Y: 67%).

### Synthesis of Intermediate 11: 4-Fluorosulfonylacetic acid.

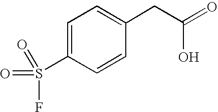

18-crown-6 (2.6 mg, 1 mol %) was added to a solution of 4-chlorosulfonylphenylacetic acid (230 mg, 0.98 mmol) and KF (113.7 mg, 1.96 mmol) in ACN (500 μL) and stirred for 4 hours. The product was then drowned out by the addition of water and extracted several times with DCM to afford desired intermediate (107 mg, Y: 50%).

### Synthesis of Intermediate 12: (4-sulfamoyl-phenyl)-acetic acid

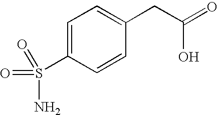

To a solution of 4-fluorosulfonylacetic acid (3 mmol) in DCM were added ammonium hydroxide (2 eq) and triethylamine (1.3 eq). The reaction mixture was stirred overnight at RT. The crude was then applied to an ISOLUTE® PEAX column and washed with ACN and with a solution of 5% aqueous HCl in ACN to afford the desired intermediate.

### Synthesis of Intermediate 13: (4-dimethylsulfamoyl-phenyl)-acetic acid

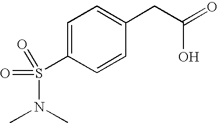

To a solution of 4-fluorosulfonylacetic acid (3 mmol) in DCM were added N,N-dimethylamine in THF solution (2 eq) and triethylamine (1.3 eq). The reaction mixture was stirred overnight at RT. The crude was then transferred to an ISOLUTE® PEA-X column and washed with ACN and with a solution of 5% aqueous HCl in ACN to afford the title intermediate.

### Synthesis of Intermediate 14: 4-[2-(4-cyano-phenyl)-acetylamino]-piperidine-1-carboxylic acid tert-butyl ester

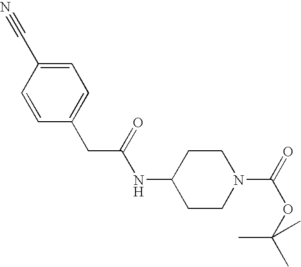

To a solution of 4-cyano-phenylacetic acid (724 mg, 4.49 mmol) in DMF (15 mL) were added while stirring HOBt (774 mg; 5.05 mmol), TBTU (1.62 g; 5.05 mmol), 4-amino-piperidine-1-carboxylic acid tert-butyl ester (750 mg; 3.74 mmol) in ACN (15 ml) followed by TEA (2.08 mL; 15 mmol). The reaction mixture was stirred overnight. The resulting mixture was concentrated under vacuum. The residue was dissolved in DCM and washed with an aqueous solution of NaHCO₃ and water. The organic phase was transferred to an ISOLUTE® PEA-X/SCX2 column and washed with DCM. The organic fraction was then concentrated under reduced pressure to afford the title intermediate as a yellow solid (838 mg; Y: 65%).

MS: (M+H)⁺=288 T_{R}=3.05 min.

### Synthesis of Intermediate 15: 2-(4-cyano-phenyl)-N-piperidin-4-yl-acetamide

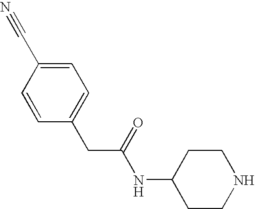

To a solution of 4-[2-(4-cyano-phenyl)-acetylamino]-piperidine-1-carboxylic acid tert-butyl ester (838 mg, 2.44 mmol) in ethanol (6.5 mL), was added hydrogen chloride in dioxane (4M solution) (9 mL, 36.6 mmol, 15 eq). The reaction mixture was stirred overnight at RT. The reaction mixture was then filtered and the solid was washed with Et₂O. The solid was dissolved in water the aqueous phase was basified using NaHCO₃ and extracted with DCM. The organic phase was concentrated under vacuum to afford the title intermediate as a solid (115 mg; Y: 20%).

MS: (M+H)⁺=244, T_{R}=1.59 min.

### Synthesis of Intermediate 16: 2-(4-cyano-phenyl)-N-[1-(2-hydroxy-ethyl)-piperidin-4-yl]-acetamide

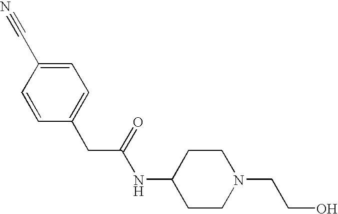

To a solution of 2-(4-cyano-phenyl)-N-piperidin-4-yl-acetamide (700 mg, 2.88 mmol) in ACN (19 mL) were added 2-chloroethanol (232 mg; 2.88 mmol), powdered sodium iodide (432 mg; 2.88 mmol) and DIEA (952 μL; 5.76 mmol). The mixture was heated at 150° C. for 25 minutes then filtered over silica gel and concentrated under reduced pressure to afford the title intermediate.

### Synthesis of Intermediate 17: N-[1-(2-chloro-ethyl)-piperidin-4-yl]-2-(4-cyano-phenyl)-acetamide hydrochloride

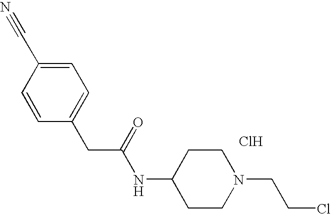

To a solution of 2-(4-cyano-phenyl)-N-[1-(2-hydroxy-ethyl)-piperidin-4-yl]-acetamide (600 mg; 2.09 mmol) in anhydrous toluene (1 ml) was added while stirring a solution of thionyl chloride (305 μL, 4.18 mmol) in anhydrous toluene (1 mL) so that the temperature remained between 25 and 30° C. The reaction was stirred overnight at RT and then concentrated under reduced pressure. The hydrochloride salt was scratched in Et₂O, filtered and washed with Et₂O to afford the title intermediate.

### Synthesis of Intermediate 18: 2-(4-cyano-phenyl)-N-[1-(2-phenylamino-ethyl)-piperidin-4-yl]-acetamide

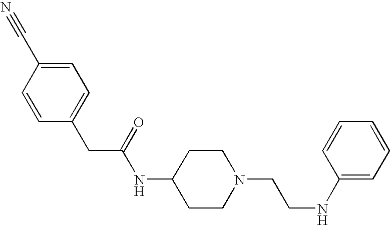

A mixture of N-[1-(2-chloro-ethyl)-piperidin-4-yl]-2-(4-cyano-phenyl)-acetamide (200 mg, 0.59 mmol), aniline (109 mg, 1.17 mmol), powdered sodium iodide (88.4 mg, 0.59 mmol) and DIEA (204 μL, 1.18 mmol) in acetonitrile (1 ml) were heated at 100° C. for 10 minutes in a microwave. The crude reaction mixture was filtered through a cotton wool plug and concentrated under vacuum. The residue was purified by silica gel chromatography (eluent: EtOAc/Cyclohexane) to afford after dry evaporation under vacuum the title intermediate.

### Synthesis of Intermediate 19; 2-(4-cyano-phenyl)-N-[1-(2-hydroxy-1-methyl-ethyl)-piperidin-4-yl]-acetamide

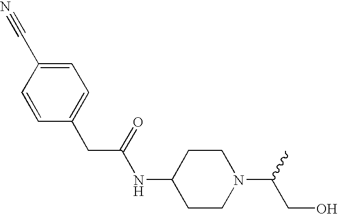

To a solution of 2-(4-cyano-phenyl)-N-piperidin-4-yl-acetamide (700 mg, 2.88 mmol) in ACN (19 mL) were added 2-chloro-propan-1-ol (271 mg; 2.88 mmol), powdered sodium iodide (432 mg; 2.88 mmol) and DIEA (952 μL; 5.76 mmol). The mixture was heated at 150° C. for 25 minutes then filtered over silica gel and concentrated under reduced pressure to afford the title intermediate.

### Synthesis of Intermediate 20: N-[1-(2-chloro-1-methyl-ethyl)-piperidin-4-yl]-2-(4-cyano-phenyl)-acetamide

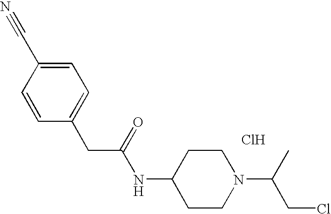

To a solution of 2-(4-cyano-phenyl)-N-[1-(2-hydroxy-1-methyl-ethyl)-piperidin-4-yl]-acetamide (629 mg; 2.09 mmol) in anhydrous toluene (1 ml) was added while stirring a solution of thionyl chloride (305 μL, 4.18 mmol) in anhydrous toluene (1 mL) so that the temperature remained between 25 and 30° C. The reaction was stirred overnight at RT and then concentrated under reduced pressure. The hydrochloride salt was scratched in Et₂O, filtered and washed with Et₂O to afford the title intermediate.

### General Method A:

A solution of HOBt (17.2 mg; 0.11 mmol) and TBTU (36 mg; 0.11 mmol) in DMF (0.5 ml) was added to a solution of 1-(3,3-diphenyl-propyl)-4-amino-piperidine dihydrochloride salt (30 mg; 0.08 mmol) and the corresponding carboxylic acid (0.1 mmol) in DMF (0.5 ml) followed by TEA (68 μL; 0.49 mmol). The reaction mixture was stirred overnight. The resulting mixture was evaporated under vacuum. The residue was dissolved in DCM and washed with an aqueous solution of NaHCO₃ and water. The organic phase was transferred to an ISOLUTE® SCX2 column and washed with MeOH and with a solution of 5% aqueous ammonia in MeOH to afford the title compounds (Table 3).

TABLE 3

| Compound | Structure |
|---|---|
| 1 | 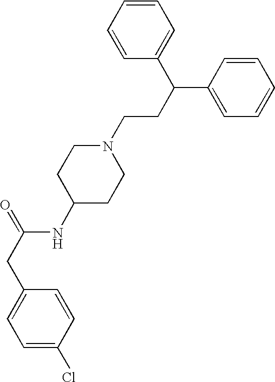 |
| 2 | 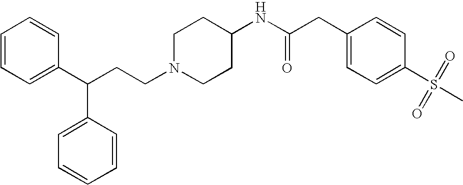 |
| 3 | 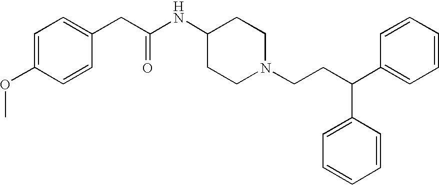 |
| 4 | 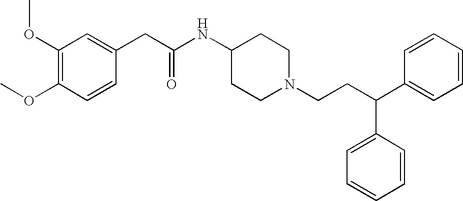 |
| 5 | 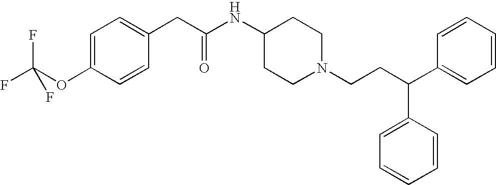 |
| 6 | 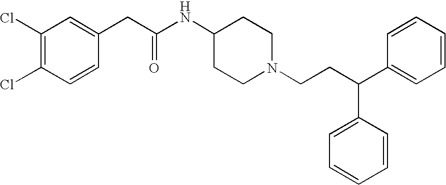 |
| 7 | 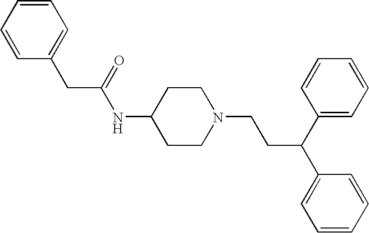 |
| 8 | 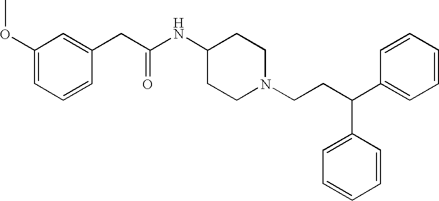 |
| 9 | 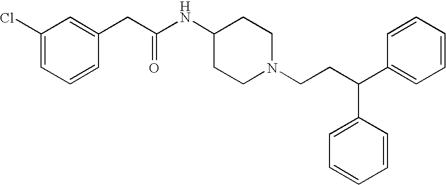 |
| 10 | 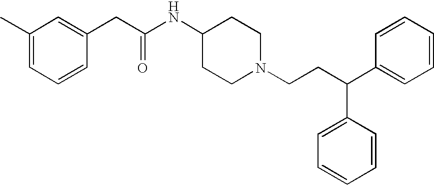 |
| 11 | 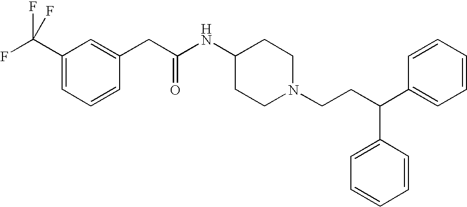 |
| 12 | 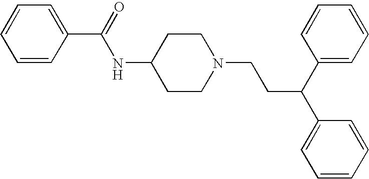 |
| 13 | 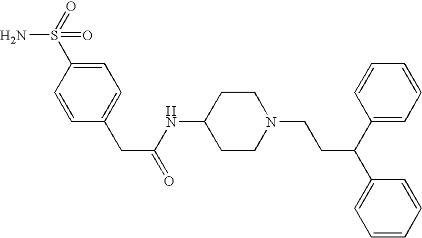 |
| 14 | 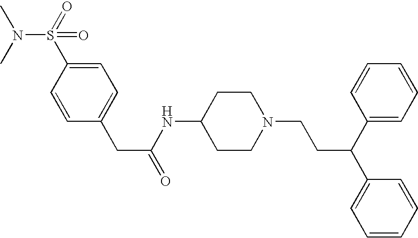 |
| 15 | 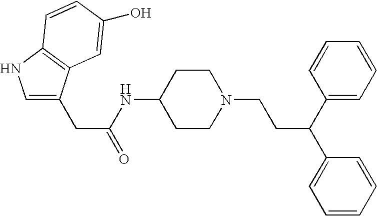 |
| 16 | 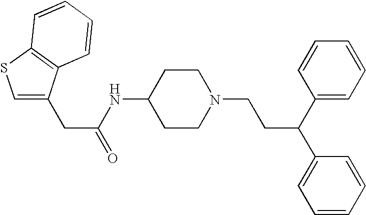 |
| 17 | 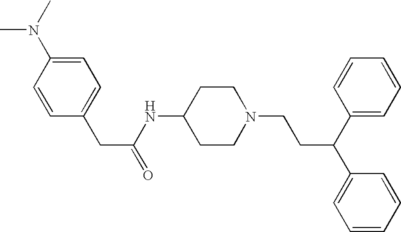 |
| 18 | 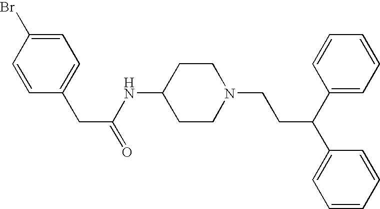 |
| 19 | 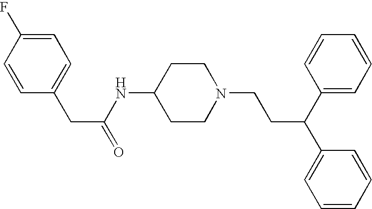 |
| 20 | 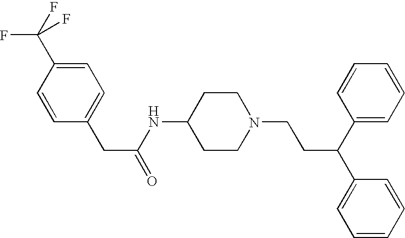 |
| 21 | 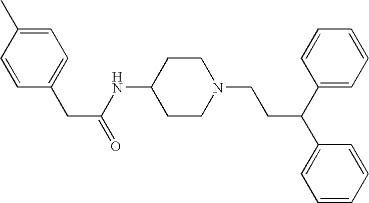 |
| 22 | 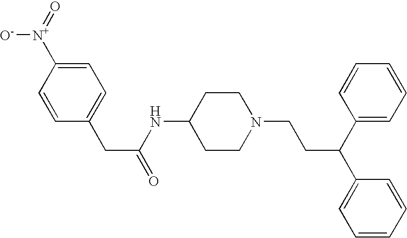 |
| 23 | 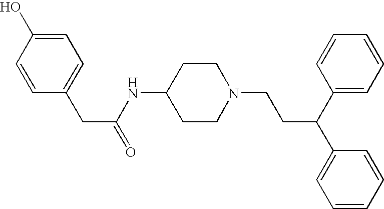 |
| 24 | 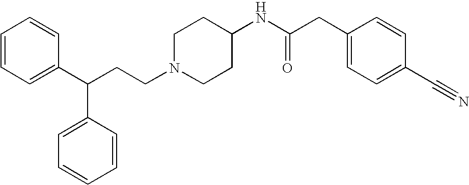 |
| 25 | 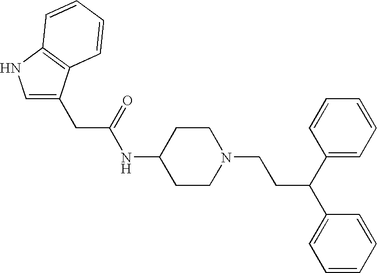 |
| 26 | 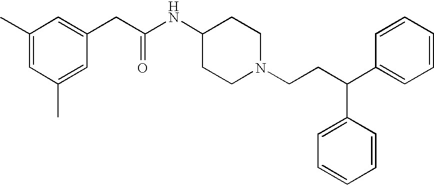 |
| 27 | 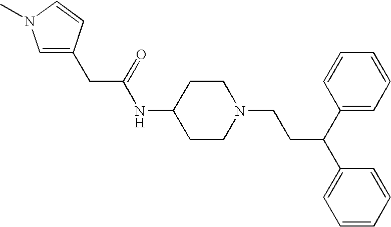 |
| 28 | 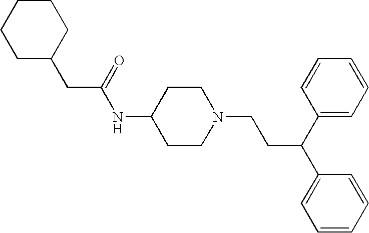 |
| 29 | 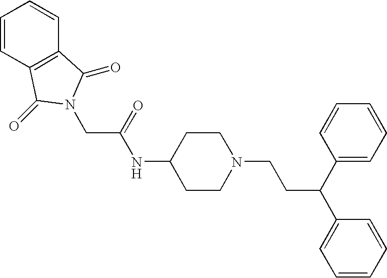 |
| 30 | 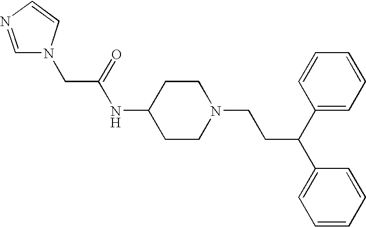 |
| 31 | 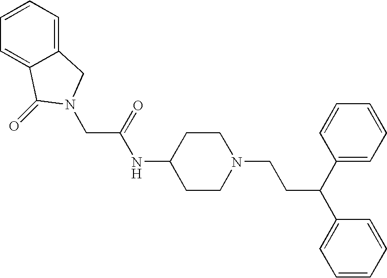 |
| 32 | 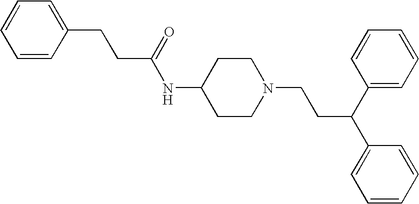 |
| 33 | 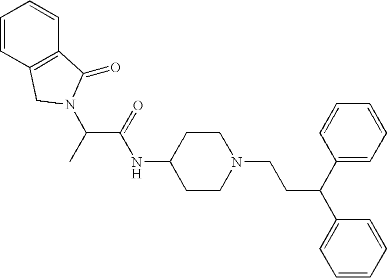 |
| 34 | 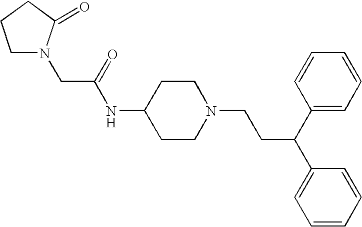 |
| 35 | 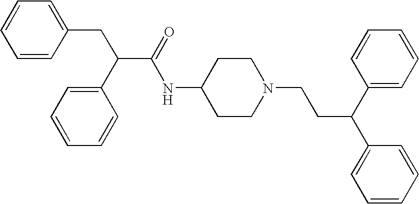 |
| 36 | 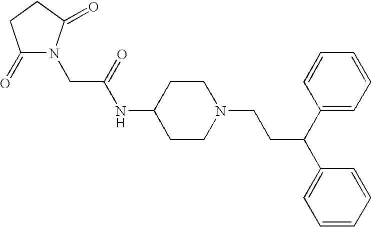 |
| 37 | 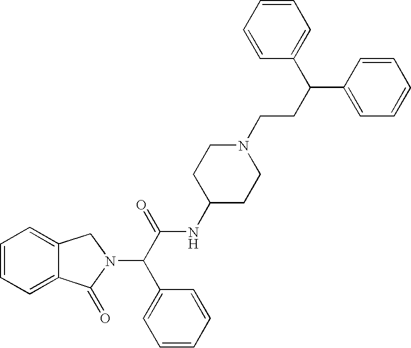 |
| 38 | 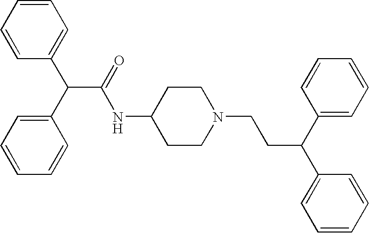 |
| 39 | 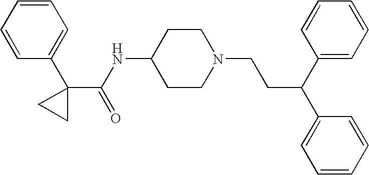 |
| 40 | 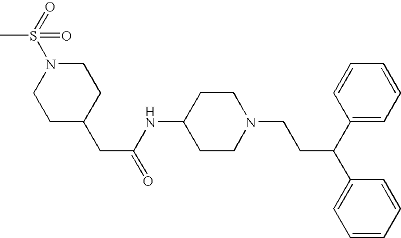 |
| 41 | 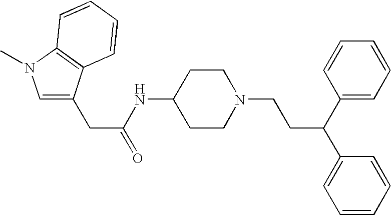 |
| 42 | 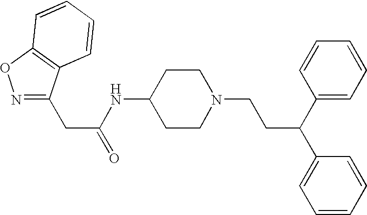 |
| 43 | 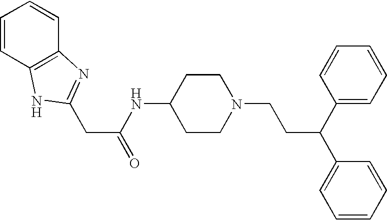 |
| 44 | 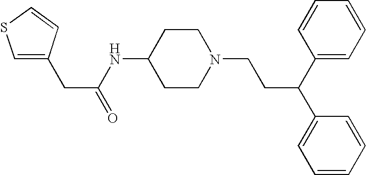 |
| 45 | 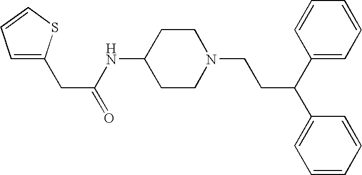 |
| 46 | 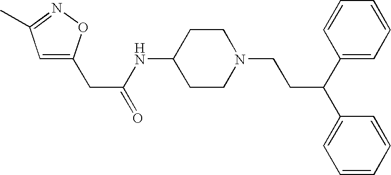 |
| 47 | 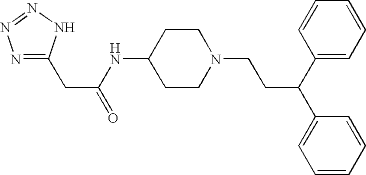 |
| 48 | 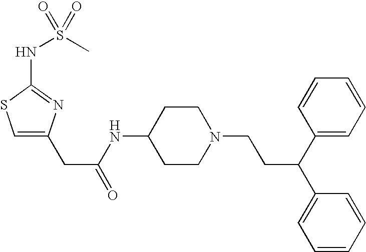 |
| 49 | 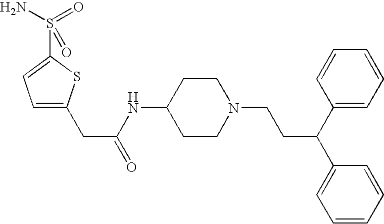 |
| 50 | 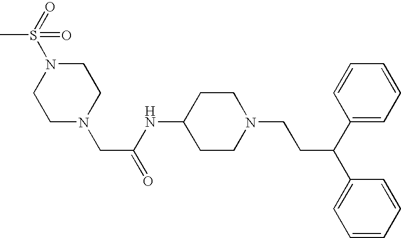 |
| 51 | 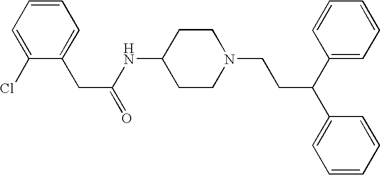 |
| 52 | 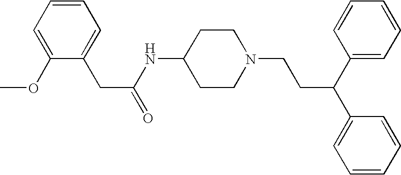 |
| 53 | 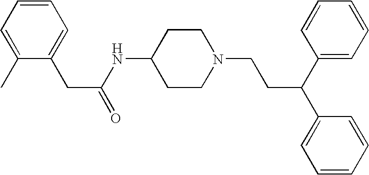 |
| 54 | 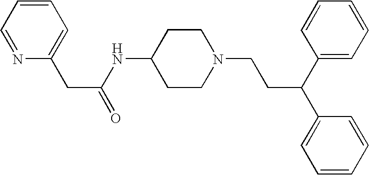 |
| 55 | 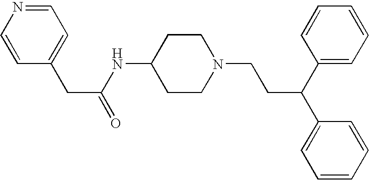 |
| 56 | 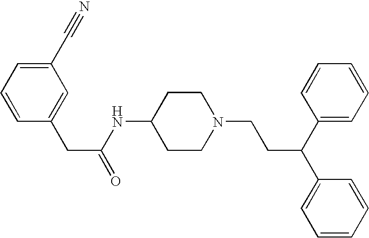 |
| 57 | 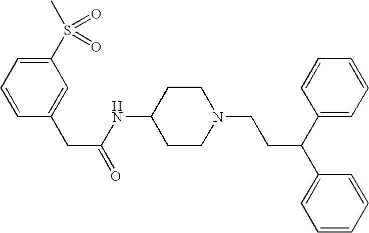 |
| 58 | 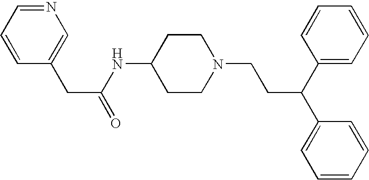 |
| 59 | 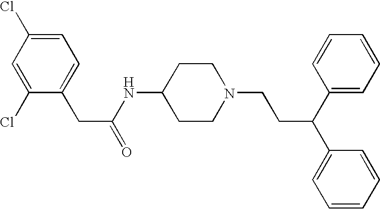 |
| 60 | 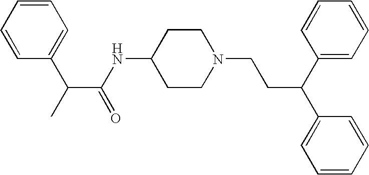 |

### General Method B:

A solution of HOBt (17.6 mg; 0.11 mmol) and TBTU (37 mg; 0.11 mmol) in DMF (0.5 ml) was added to a solution of 1-(3,3-diphenyl-propyl)-4-N-methyl-amino-piperidine (25 mg; 0.08 mmol) and the acid (0.1 mmol) in DMF (1.5 ml) followed by TEA (55 μL; 0.4 mmol). The reaction mixture was stirred overnight. The resulting mixture was evaporated under vacuum. The residue was dissolved in DCM and washed with an aqueous solution of NaHCO₃ and water. The organic phase was transferred to an ISOLUTE® SCX2 column and washed with MeOH and with a solution of 5% aqueous ammonia in MeOH to afford the title compounds (Table 4).

TABLE 4

| Com- | |
|---|---|
| pound | Structure |
| 61 | 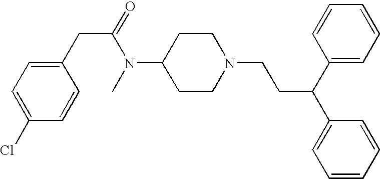 |
| 62 | 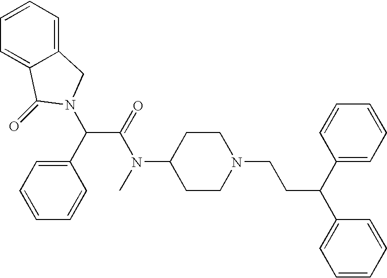 |
| 63 | 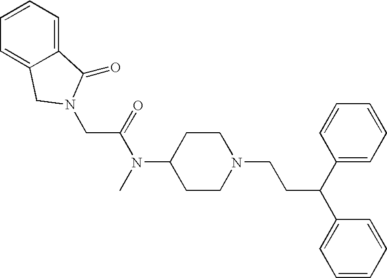 |

### General Method C:

A solution of HOBt (8.3 mg; 0.05 mmol) and TBTU (17.4 mg; 0.05 mmol) in DMF (0.5 ml) was added to a solution of 1-(1-methyl-3,3-diphenyl-propyl)-4-amino-piperidine dihydrochloride salt (15 mg; 0.04 mmol) and the acid (0.05 mmol) in DMF (0.5 ml) followed by TEA (41 μL; 0.29 mmol). The reaction mixture was stirred overnight. The resulting mixture was evaporated under vacuum. The residue was dissolved in DCM and washed with an aqueous solution of NaHCO₃ and water. The organic phase was transferred to an ISOLUTE® SCX2 column and washed with MeOH and with a solution of 5% aqueous ammonia in MeOH to afford the title compounds (Table 5).

TABLE 5

| Compound | Structure |
|---|---|
| 64 | 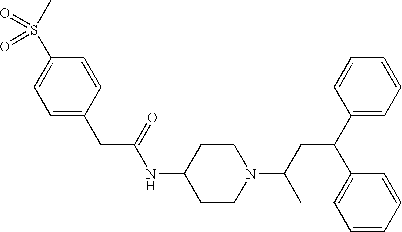 |
| 65 | 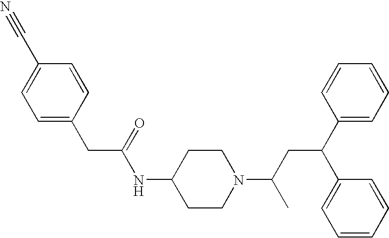 |

### General Method D:

To a mixture of 2-(4-cyano-phenyl)-N-[1-(2-phenylamino-ethyl)-piperidin-4-yl]-acetamide (40 mg, 0.11 mmol), TEA (30.5 μL, 0.22 mmol) in acetonitrile (1 ml) was added dropwise at 0° C. to the corresponding alkyl halide (0.1 mmol) The mixture was then stirred overnight at 70° C. The crude reaction mixture was concentrated under reduced pressure, DCM was then added and the organic layer was washed with water, dried over MgSO₄ and concentrated under reduce pressure. The residue was purified by silica gel chromatography (eluent: DCM/MeOH) to afford after dry evaporation under vacuum the title compounds. (Table 6)

TABLE 6

| Compound | Structure |
|---|---|
| 66 | 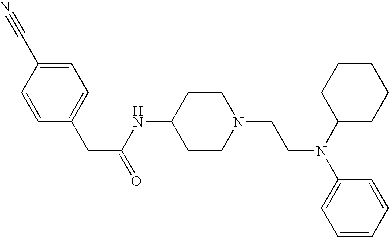 |
| 67 | 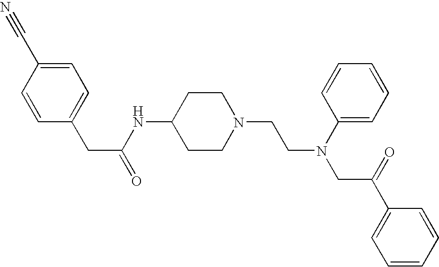 |

### General Method E:

A suspension of aryl boronic acid (2.000 mmol), Cu(OAc)₂.H₂O (0.020 g, 0.100 mmol), and powdered 4 Å molecular sieves (0.75 g) in DCM (8 ml) was stirred for 5 minutes at room temperature. To this stirring suspension was added 2-(4-cyano-phenyl)-N-[1-(2-phenylamino-ethyl)-piperidin-4-yl]-acetamide (0.362 g, 1 mmol). The reaction mixture was then sealed with a rubber septa, and stirred under an atmosphere of O₂. The reaction was stirred with a magnetic stir bar for a period of 24H. The crude was filtered through a plug of celite to remove the molecular sieves and any insoluble products and the organic layer was concentrated using a rotary evaporator to afford the crude mixture. The product was isolated on SPE-SCX. (Table 7)

TABLE 7

| Compound | Structure |
|---|---|
| 68 | 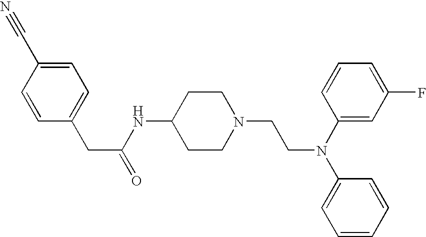 |
| 69 | 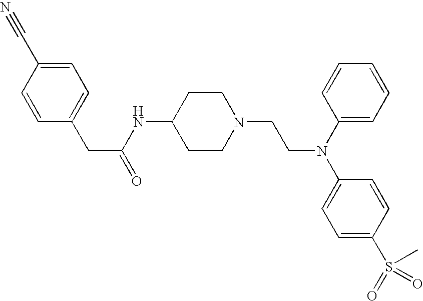 |
| 70 | 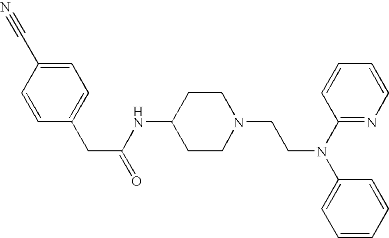 |

### Synthesis of Compound 71: N-[1-(3-cyano-3,3-diphenylpropyl)-piperidin-4-yl]-2-(4-cyano-phenyl)-acetamide

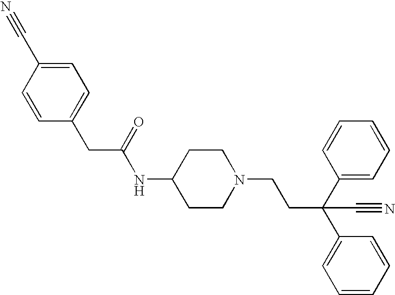

To a solution of 2-(4-cyano-phenyl)-N-piperidin-4-yl-acetamide (40 mg; 0.16 mmol) in ACN (2.5 ml) were added 3,3-diphenylpropyl bromide (59.2 mg; 0.2 mmol), tetrabutylammonium iodide (3.1 mg; 0.016 mmol) and potassium carbonate (66.3 mg; 0.48 mmol). The mixture was heated at 100° C. 10 days long. The reaction mixture was cooled and filtered over silica gel and concentrated. The residue was purified by silica gel chromatography (eluent: EtOAc) to afford after dry evaporation under vacuum the title compound.

### Synthesis of Compound 72: 2-(4-cyano-phenyl)-N-[1-(2-diphenylamino-ethyl)-piperidin-4-yl]-acetamide

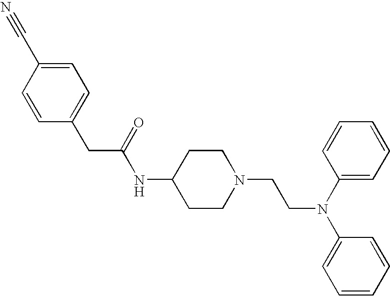

A mixture of N-[1-(2-chloro-ethyl)-piperidin-4-yl]-2-(4-cyano-phenyl)-acetamide (40 mg, 0.13 mmol), diphenylamine (44.2 mg, 0.26 mmol), powdered sodium iodide (19.5 mg, 0.13 mmol) and DIEA (45.3 μL, 0.26 mmol) in acetonitrile (0.5 ml) were heated at 100° C. for 10 minutes under microwave irradiation. The crude reaction mixture was filtered through a cotton wool plug and concentrated under vacuum. The residue was purified by silica gel chromatography (eluent: EtOAc/Cyclohexane) to afford after dry evaporation under vacuum the title compound.

### Synthesis of Compound 73: 2-(4-cyano-phenyl)-N-[1-(2-diphenylamino-1-methyl-ethyl)-piperidin-4-yl]-acetamide

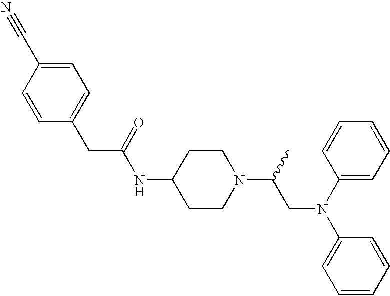

A mixture of N-[1-(2-chloro-1-methyl-ethyl)-piperidin-4-yl]-2-(4-cyano-phenyl)-acetamide (46 mg, 0.13 mmol), diphenylamine (44.2 mg, 0.26 mmol), powdered sodium iodide (19.5 mg, 0.13 mmol) and DIEA (45.3 μL, 0.26 mmol) in ACN (0.5 ml) were heated at 100° C. for 10 minutes in a microwave. The crude reaction mixture was filtered through a cotton wool plug and concentrated under vacuum. The residue was purified by silica gel chromatography (eluent: EtOAc/Cyclohexane) to afford after dry evaporation under vacuum the title compound.

### Synthesis of Compound 74: cyclohexanecarboxylic acid (2-{4-[2-(4-cyano-phenyl)-acetylamino]-piperidin-1-yl}-ethyl)-phenyl-amide

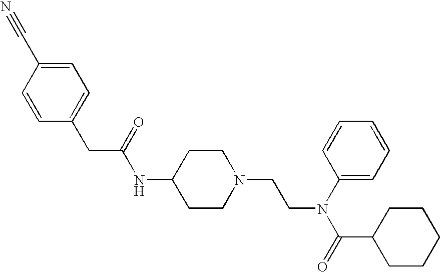

To a mixture of 2-(4-cyano-phenyl)-N-[1-(2-phenylamino-ethyl)-piperidin-4-yl]-acetamide (40 mg, 0.11 mmol), TEA (30.5 μL, 0.2.2 mmol) in DCM (1 ml) was added dropwise at 0° C. the cyclohexanecarbonyl chloride (14.8 μL, 0.11 mmol). The mixture was then stirred overnight at RT. The crude reaction mixture was washed with a saturated aqueous NaHCO₃ solution, dried over MgSO₄ and concentrated under reduced pressure. The residue was purified by silica gel chromatography (eluent: DCM/MeOH) to afford after dry evaporation under vacuum the title compound.

### Synthesis of Compound 75: 4-[2-(4-Cyano-phenyl)-acetylamino]-1-(3,3-diphenyl-propyl)-1-methyl-piperidinium iodide

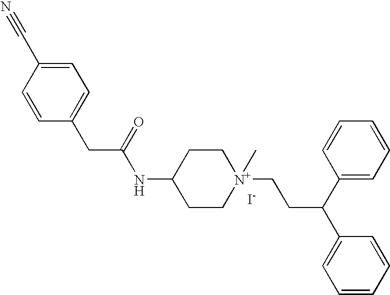

To a solution of 2-(4-cyano-phenyl)-N-[1-(3,3-diphenyl-propyl)-piperidin-4-yl]-acetamide (50 mg; 0.11 mmol) in 2 mL of DCM, was added methyl iodide (142 μL, 2.28 mmol, 20 eq). The reaction mixture was refluxed for 4.5 hours, then concentrated under vacuum to afford the title compound (29.5 mg; Y: 57%).

MS: (M+H)⁺=453, T_{R}=2.73 min.

### Synthesis of Compound 76: 4-([1-(3,3-diphenyl-propyl)-piperidin-4-ylcarbamoyl]-methyl)-benzamide

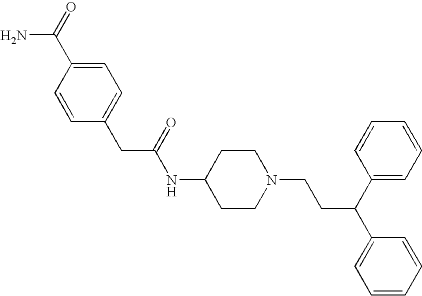

2-(4-cyano-phenyl)-N-[1-(3,3-diphenyl-propyl)-piperidin-4-yl]-acetamide (40 mg, 0.091 mmol) was reacted with sulfuric acid (778 μL, 0.015 mmol) in water (390 μL) for 4 hours at RT. The reaction was stopped and basified with a saturated aqueous NaHCO₃ solution, extracted several time with EtOAc and concentrated under reduced pressure to afford the title compound.

### Synthesis of Compound 77: N-[1-(3,3-diphenyl-propyl)-piperidin-4-yl]-2-(2-methyl-2H-tetrazol-5-yl)-acetamide

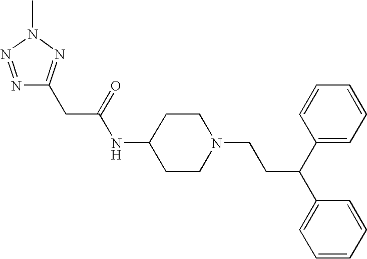

To a solution of N-[1-(3,3-diphenyl-propyl)-piperidin-4-yl]-2-(2H-tetrazol-5-yl)-acetamide (40 mg, 0.1 mmol) and sodium hydroxide (7.9 mg, 0.2 mmol) in THF (2 mL) was added methyl iodide (12 μL, 0.2 mmol); The reaction mixture was stirred for 4 hours at RT and then concentrated under reduced pressure. The residue was purified by silica gel chromatography (eluent: DCM/MeOH) to afford after dry evaporation the title compound.

### Synthesis of Compound 78: N-[1-(3,3-diphenyl-propyl)-piperidin-4-yl]-2-(1-methyl-1H-tetrazol-5-yl)-acetamide

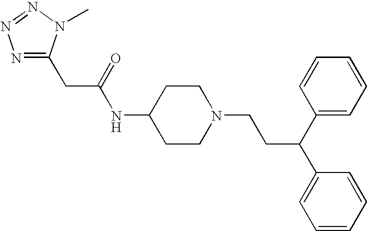

To a solution of N-[1-(3,3-diphenyl-propyl)-piperidin-4-yl]-2-(2H-tetrazol-5-yl)-acetamide (40 mg, 0.1 mmol) and sodium hydroxide (7.9 mg, 0.2 mmol) in THF (2 mL) was added methyl iodide (12 μL, 0.2 mmol). The reaction mixture was stirred for 4 hours at RT and then concentrated under reduced pressure. The residue was purified by silica gel chromatography (eluent: DCM/MeOH) to afford after dry evaporation the title compound.

### Synthesis of Compounds 79 to 127 and 133

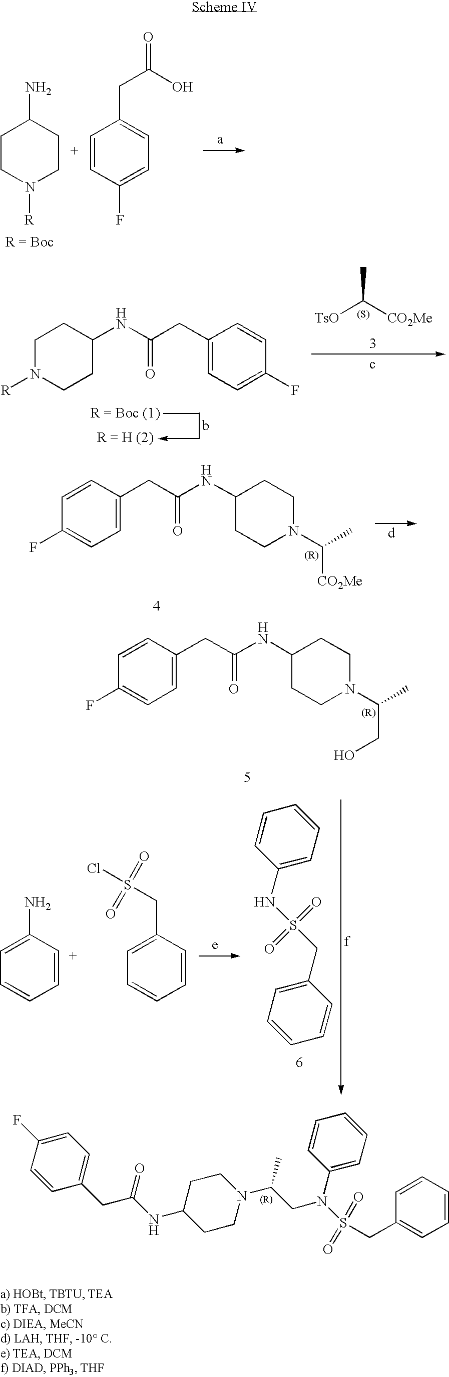

### Synthesis of Intermediate 21: tert-butyl 4-(2-(4-fluorophenyl)acetamido)piperidine-1-carboxylate

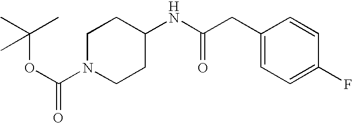

To a solution of HOBt (26.4 mmol; 1.2 eq) and TBTU (26.4 mmol; 1.2 eq) was added the 2-(4-fluorophenyl)acetic acid (26 mmol; 1.2 eq) in. DMF (200 mL) followed by the addition of TEA (26.4 mmol; 1.2 eq) and tert-butyl 4-aminopiperidine-1-carboxylate (22 mmol; 1 eq). The reaction mixture was stirred at RT overnight. After concentrating the reaction mixture under reduced pressure, the residue was taken up in DCM and washed three times with saturated NaHCO3 solution. The organic phase was separated, dried (over MgSO4) and after filtration, the volatiles were removed under reduced pressure. The solid thus obtained was triturated with diethyl ether, re-filtered and dried in vacuo to obtain the title intermediate.

### Synthesis of Intermediate 22: 2-(4-fluorophenyl)-N-(piperidin-4-yl)acetamide

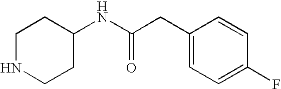

To a DCM (150 mL) solution of tert-butyl 4-(2-(4-fluorophenyl)acetamido)piperidine-1-carboxylate was added TFA (200 mmol; 10 eq) and the reaction mixture was stirred at RT for 1 h. After concentrating this reaction mixture under reduced pressure, the obtained residue was taken up in pH 14 aqueous solution (NaOH). The aqueous phase was extracted three times with EtOAc, and the combined organic phase was dried over MgSO4, filtered and the volaties were removed in vacuo. The solid thus obtained was triturated with diethyl ether, re-filtered and dried under vacuum.

### Synthesis of Intermediate 23: (S)-methyl 2-(tosyloxy)propanoate

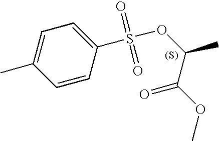

To an anhydrous DCM solution of lactate (20 mmol; 1 eq) and 4-methylbenzene-1-sulfonyl chloride (24 mmol; 1.2 eq) was added TEA (30.9 mmol; 1.55 eq) at 0° C. under inert atmosphere. The reaction mixture was stirred at RT for 1 day. After addition of water, the reaction mixture was subjected to DCM extraction. The organic phase was washed with saturated NaHCO3, dried (MgSO4) and after filtration and removal of the volatiles the title intermediate was obtained as crude product, which was further purified by silica-gel flash column chromatography (elution: 95% cyclohexane/5% EtOAc to 90% cyclohexane/10% EtOAc).

### Synthesis of Intermediate 24: (R)-methyl 2-(4-(2-(4-fluorophenyl)acetamido)piperidin-1-yl)propanoate

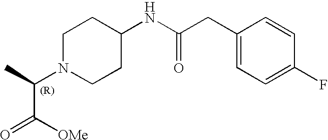

To the solution of 2-(4-fluorophenyl)-N-(piperidin-4-yl)acetamide (1.84 mmol; 1 eq) was added DIEA (2.16 mmol; 1.2 eq) and (S)-methyl 2-(tosyloxy)propanoate (1.94 mmol; 1.05 eq) in anhydrous MeCN (11 mL) under inert atmosphere. The reaction mixture was stirred under reflux for 1 day. The volatiles were then removed in vacuo and the obtained residue was taken up in DCM and washed three times with saturated NaHCO3. The organic phase was separated, dried (MgSO4) and concentrated under reduced pressure. The residue thus obtained was subjected to ISOLUTE SCX2™ column and washed with MeOH and with a solution of 5% aqueous ammonia in MeOH to obtain the title intermediate.

### Synthesis of Intermediate 25: (R)-2-(4-fluorophenyl)-N-(1-(1-hydroxypropan-2-yl)piperidin-4-yl)acetamide

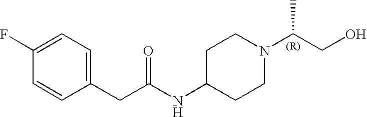

To the piperidine ester was added 1 equiv of LiAlH4 (0.93 mmol; 1 eq) in anhydrous THF (12 mL) at −10° C. under inert atmosphere. After stirring the reaction at −10° C. for 1 h, to the reaction milieu was added aqueous NaOH (8 M). The mixture thus obtained was then extracted several times with EtOAc, the organic extracts were combined, dried (MgSO4) and after filtration, the volatiles were removed in vacuo to obtain the title intermediate.

### Synthesis of Intermediate 26: N,1-diphenylmethanesulfonamide

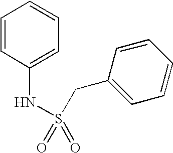

Triethylamine (3 mmol; 1.2 eq) was added to a solution containing aniline (3 mmol; 1.2 eq) and benzylsulfonyl chloride (2.5 mmol; 1 eq) in anhydrous DCM (5 mL) under inert atmosphere. The reaction mixture was then stirred at RT for 2 days whereupon HCl (1M) was added and the reaction mixture was extracted with DCM. The organic phases were combined and dried over MgSO4. After filtering the MgSO4 and removal of volatiles, a residue was obtained that was then subjected to silica-gel column chromatographic purification to afford this intermediate.

Note: All sulfonyl chlorides that were not commercially available were accessed through the procedure described in Nishiguchi et al, Synthesis, 2006, 4131 by using the commercially available halide, or via pseudohalides (mesylate, tosylate) from the alcohol precursor.

### General Method F:

### Example of (R)—N-(1-(1-(N,1-diphenylmethylsulfonamido)propan-2-yl)piperidin-4-yl)-2-(4-fluorophenyl)acetamide

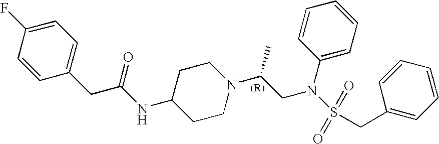

To the solution of (R)-2-(4-fluorophenyl)-N-(1-(1-hydroxypropan-2-yl)piperidin-4-yl)acetamide (0.169 mmol; 1 eq) was added PPh3 (0.254 mmol; 1.5 eq), DIAD (0.254 mmol; 1.5 eq) and N,1-diphenylmethanesulfonamide (0.203 mmol; 1.2 eq) in anhydrous THF at 0° C. under inert atmosphere. After stirring the reaction mixture for 5 min at 0° C., it was allowed to warm up to RT whereupon it was further stirred for 2-3 hours. After concentrating the reaction mixture, the crude residue was subjected to ISOLUTE SCX2™ column and washed with MeOH and with a solution of 5% aqueous ammonia in MeOH. Thereafter the residue thus purified was further subjected to silica-gel column chromatography, with cyclohexane (100%) to cyclohexane/EtOAc (3:7) as typical eluant system to afford the title compound (see Table 10).

The other compounds of table 10 were obtained in an analogous manner.

### Synthesis of Compounds 128 to 132

Compounds 128 to 132 (see Table 10) were obtained according to Scheme V.

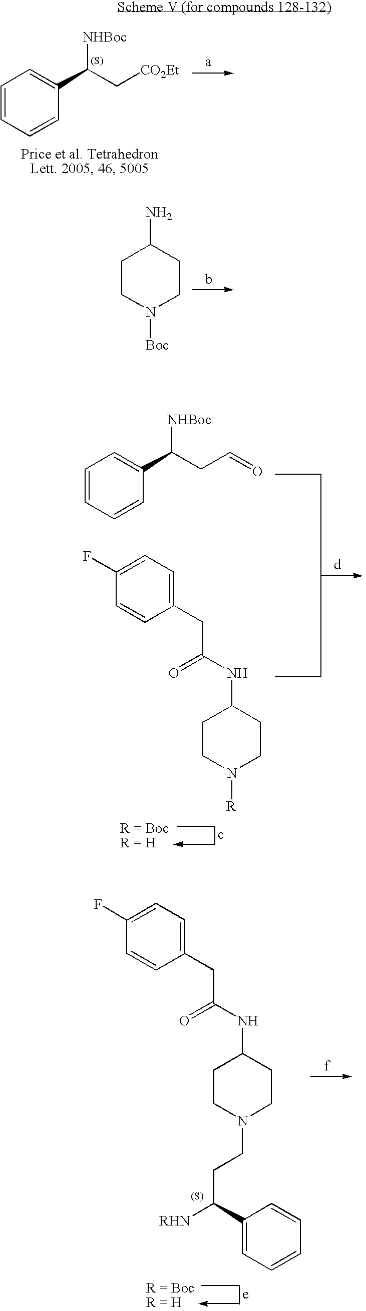

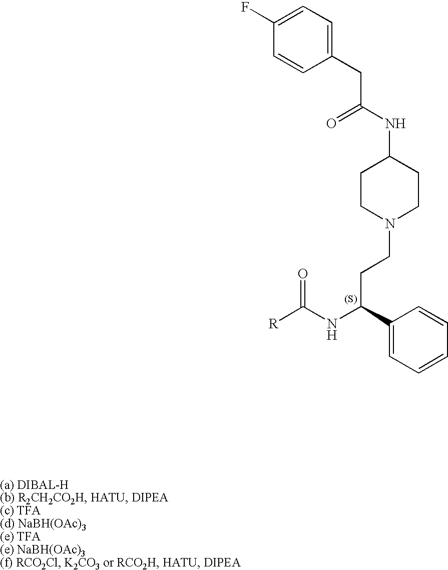

### Synthesis of Compounds 134 and 135

Compounds 134 and 135 (see Table 10) were prepared according to Scheme VI.

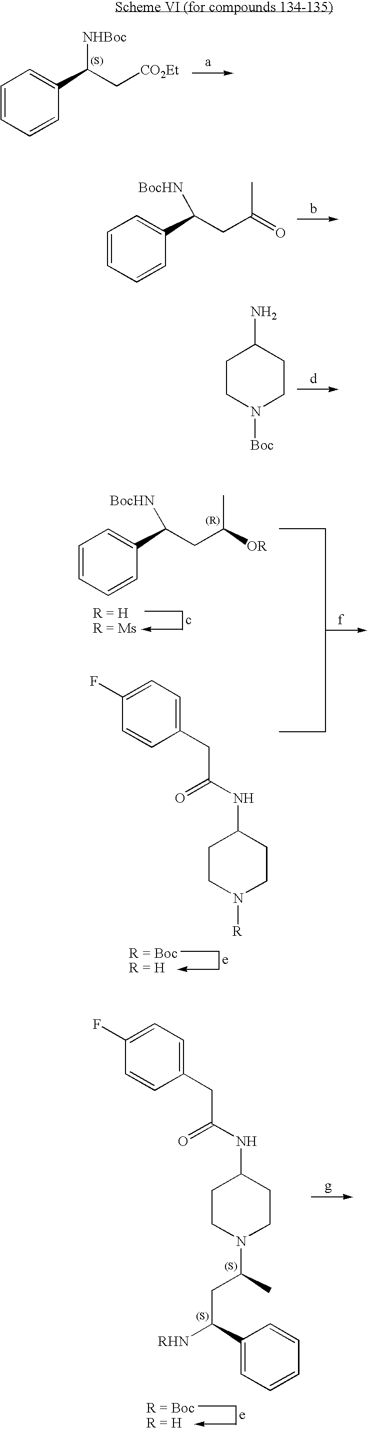

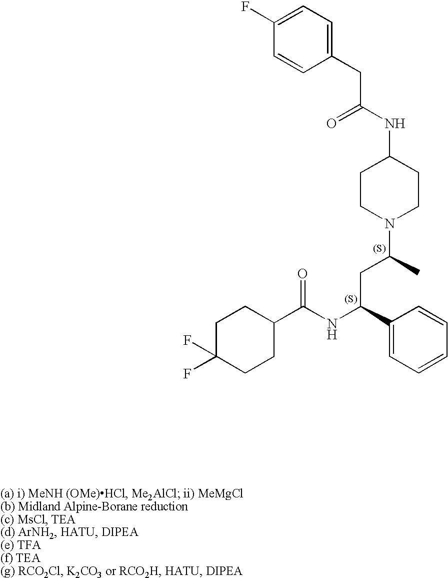

TABLE 8

| Compound | Structure |
|---|---|
| 79 | 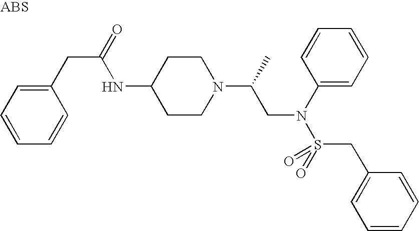 |
| 80 | 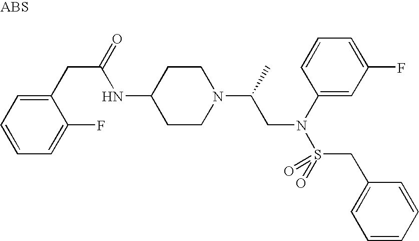 |
| 81 | 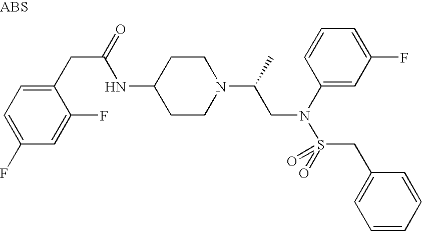 |
| 82 | 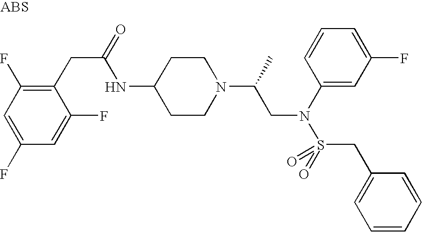 |
| 83 | 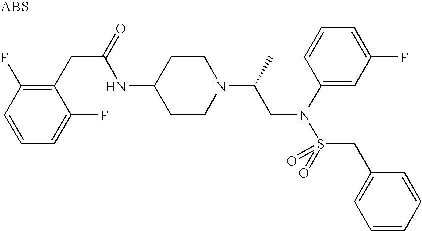 |
| 84 | 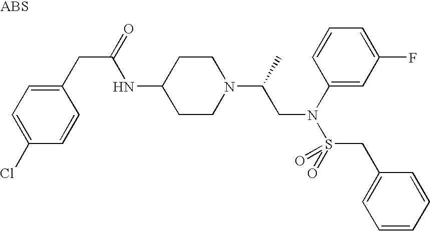 |
| 85 | 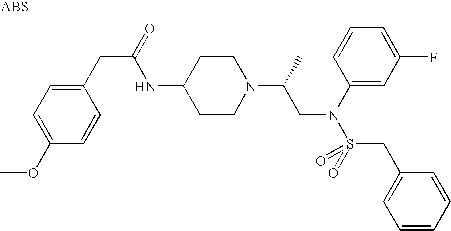 |
| 86 | 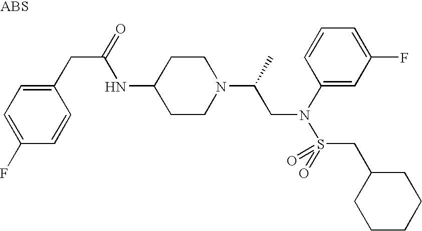 |
| 87 | 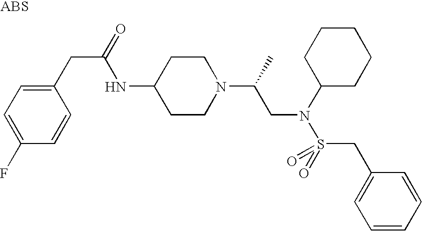 |
| 88 | 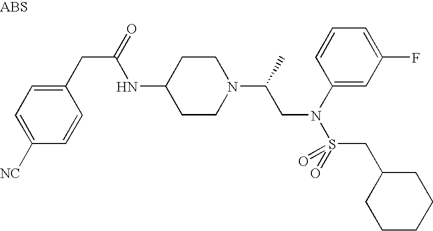 |
| 89 | 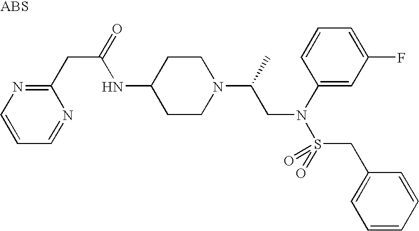 |
| 90 | 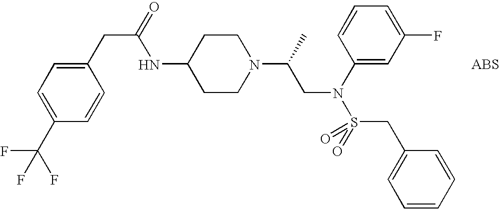 |
| 91 | 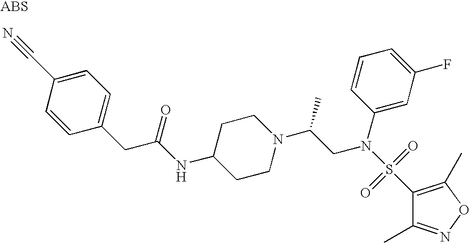 |
| 92 | 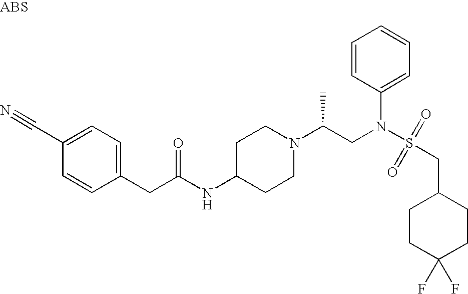 |
| 93 | 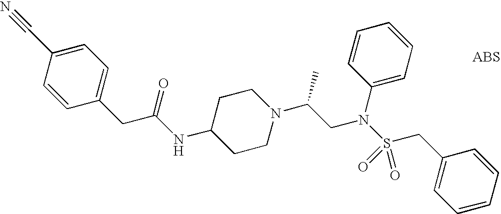 |
| 94 | 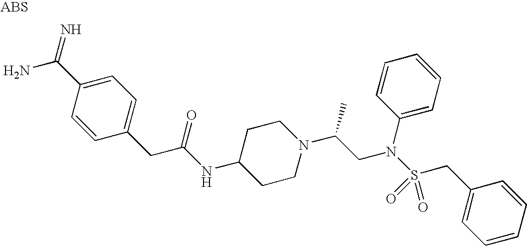 |
| 95 | 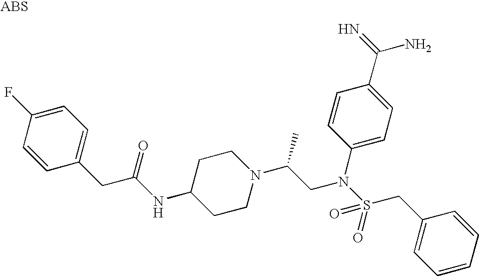 |
| 96 | 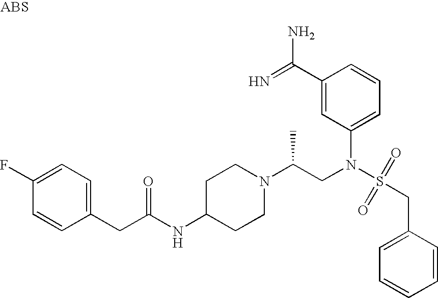 |
| 97 | 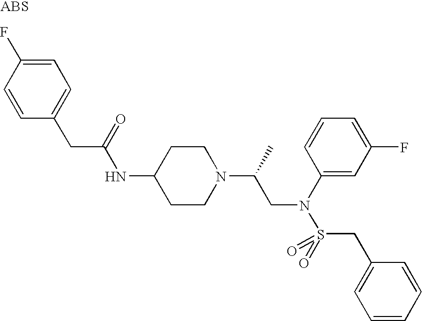 |
| 98 | 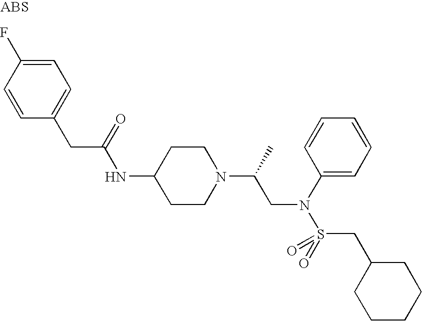 |
| 99 | 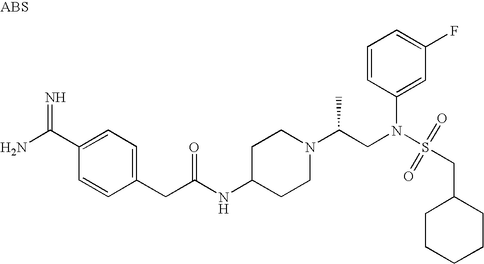 |
| 100 | 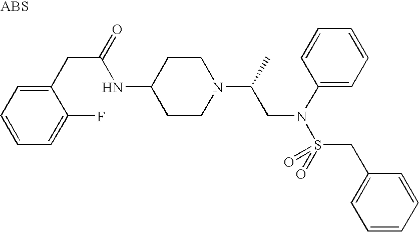 |
| 101 | 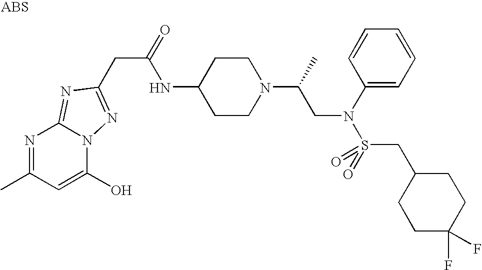 |
| 102 | 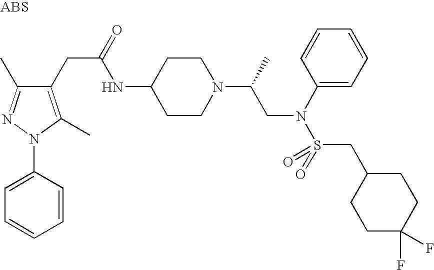 |
| 103 | 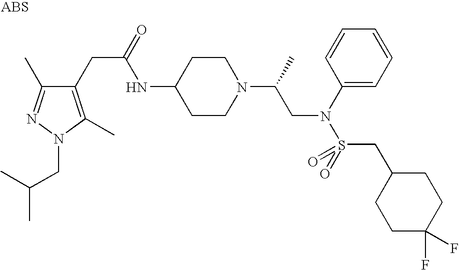 |
| 104 | 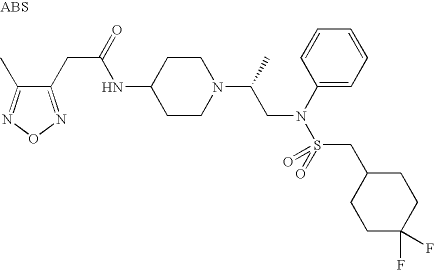 |
| 105 | 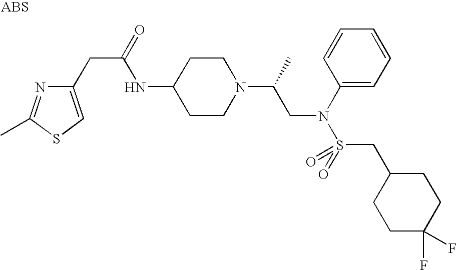 |
| 106 | 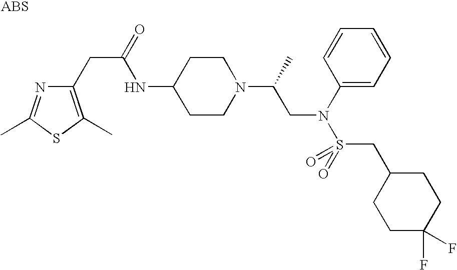 |
| 107 | 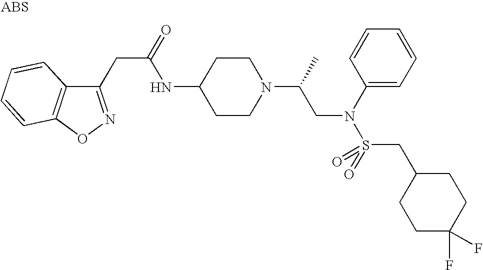 |
| 108 | 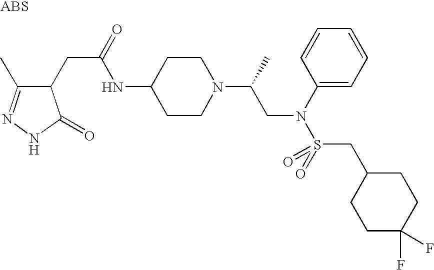 |
| 109 | 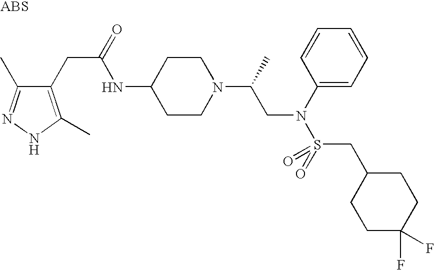 |
| 110 | 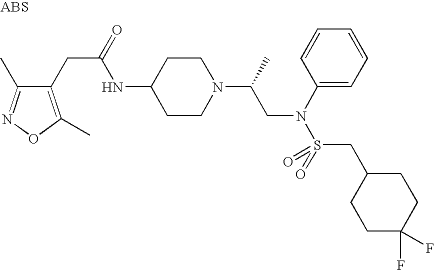 |
| 111 | 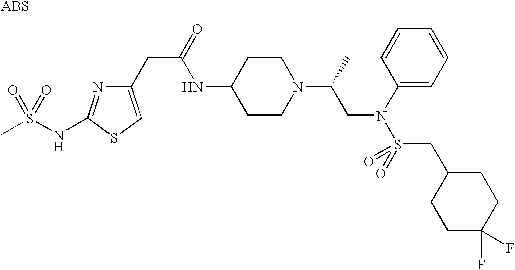 |
| 112 | 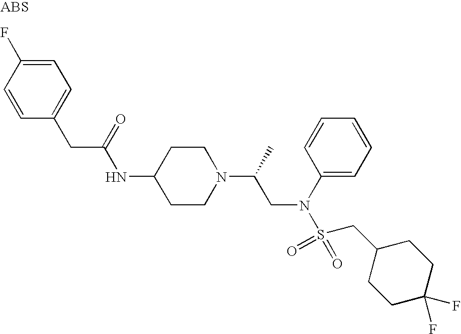 |
| 113 | 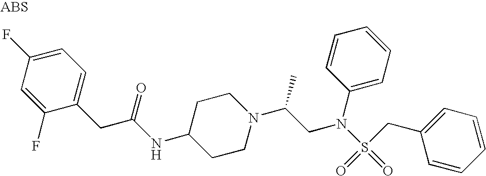 |
| 114 | 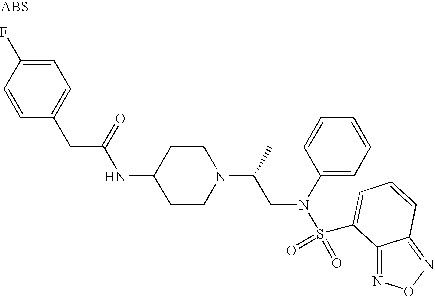 |
| 115 | 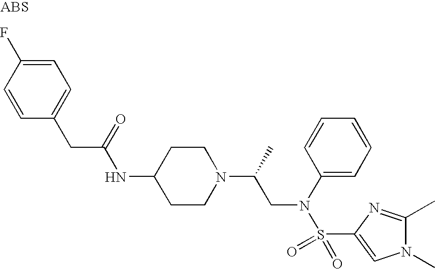 |
| 116 | 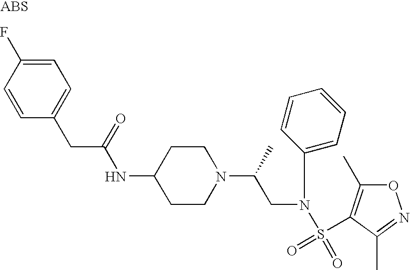 |
| 117 | 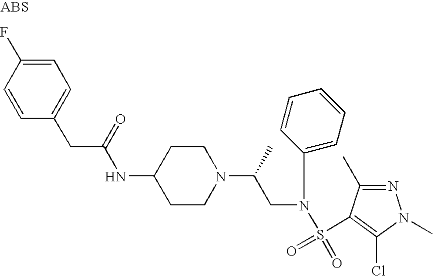 |
| 118 | 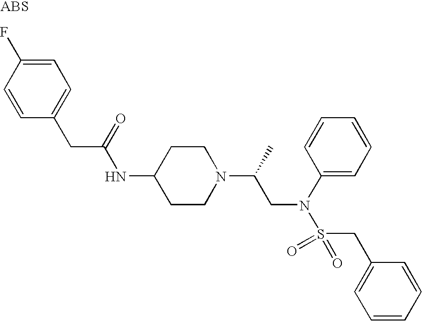 |
| 119 | 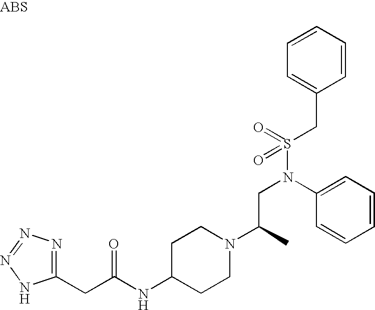 |
| 120 | 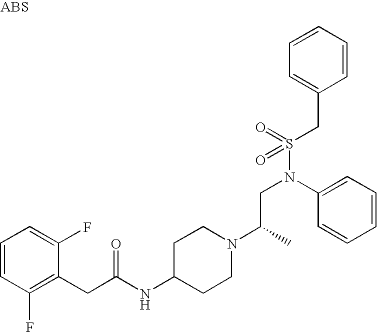 |
| 121 | 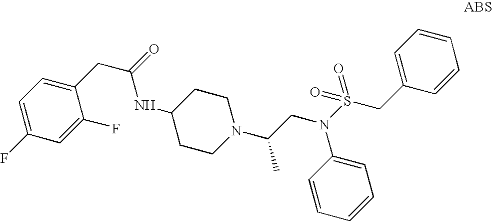 |
| 122 | 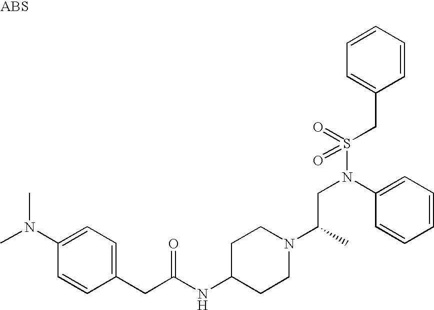 |
| 123 | 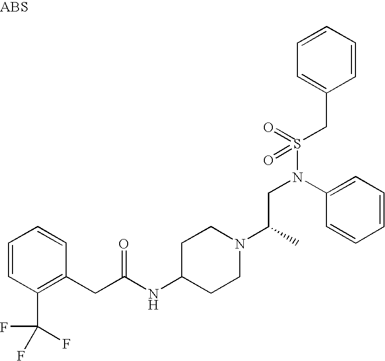 |
| 124 | 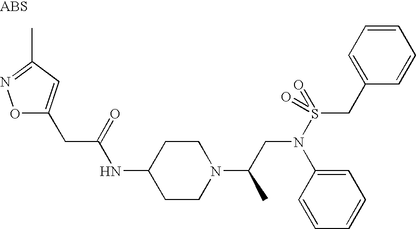 |
| 125 | 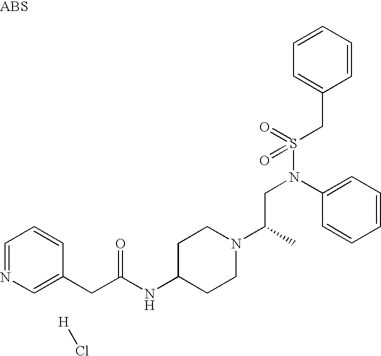 |
| 126 | 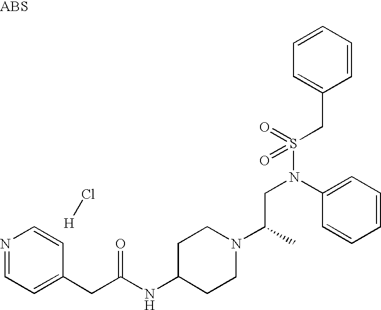 |
| 127 | 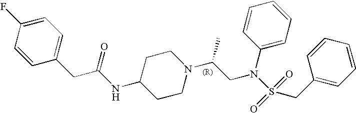 |
| 128 | 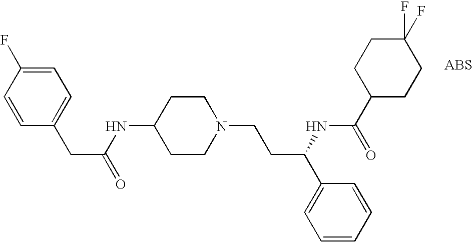 |
| 129 | 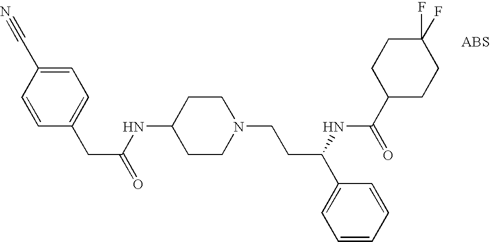 |
| 130 | 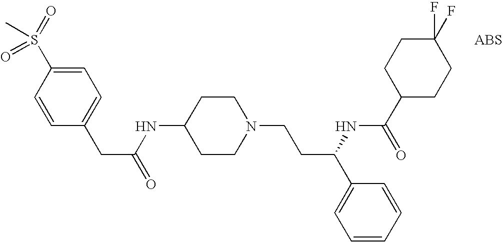 |
| 131 | 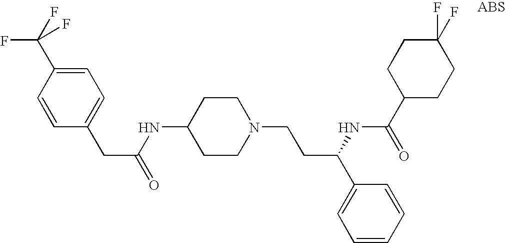 |
| 132 | 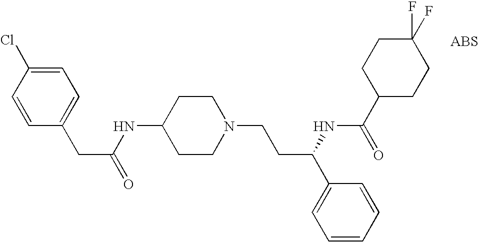 |
| 133 | 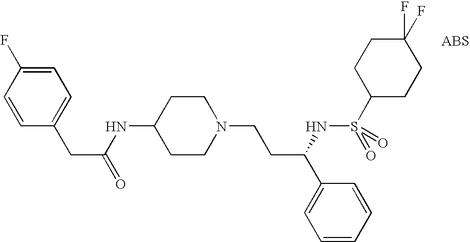 |
| 134 | 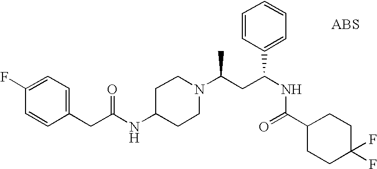 |
| 135 | 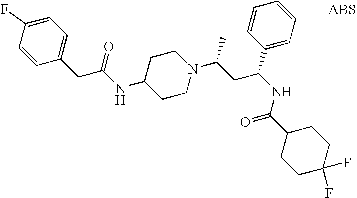 |

### BIOLOGY EXAMPLES

### Cell Based Assay: Calcium Flux. the Aequorin-Based Assay

The aequorin assay uses the responsiveness of aequorin to intracellular calcium release induced by the activation of G Protein Coupled Receptors (Stables et al., 1997, Anal. Biochem. 252:115-126; Detheux et al., 2000, J. Exp. Med., 192 1501-1508). Briefly, Chinese hamster ovary cells expressing the CCR5 receptor are transfected to coexpress apoaequorin and Gα16. Cells are incubated with 5 μM Coelenterazine H (Promega) overnight at room temperature, and resuspended at a concentration of 0.1×10⁶ cells/ml. Cells are then mixed with test agonist or antagonist compounds and light emission by the aequorin is recorded with a luminometer (FDSS 6000-Hamamatsu) for 30 sec. Results are expressed as Relative Light Units (RLU). Controls include cells not expressing CCR5 in order to exclude possible non-specific effects of the test compound.

An agonist response is defined as an increase of light emission by aequorin corresponding to 10% or more of the light emitted by a reference sample of cells expressing CCR5 and treated with a the reference agonist ligand MIP-1β. The results of the tested compounds are reported as the concentration of compound required to reach 50% (EC50) of the maximum level of light emission induced by these compounds.

When tested in the assay described above and by way of illustration the compounds no 24, 64, 65, 25, 28, 1, 2, 7, 3 and 5 have an EC50 ranging from 27.9 mM to 619.7 nM (table 9).

An antagonist response is defined as a decrease of light emission by aequorin of 10% or more in a sample of cells expressing CCR5 stimulated by a reference agonist ligand MIP-1β and treated with the compound of the invention, relative to a sample of cells expressing CCR5 but only treated with the reference agonist ligand MIP-1β.

The results of the tested compounds are reported as the concentration of compound required to inhibit 50% (IC50) of the maximum emission of light induced by the reference agonist compound.

When tested in the assay described above and by way of illustration the compound no 61 has an IC50 of 34.9 nM (table 9).

### Inhibitory Effect on HIV Infection to MAGI-CCR5 Cells

The inhibitory activity of the compounds of the invention on HIV infection is measured on the human MAGI R5 recombinant cell line coexpressing the human CCR5 receptor and CD4 at their extracellular membrane. MAGI R5 cells are plated in black view plates at 10,000 cells/well and incubated with the appropriate concentrations of the compounds of the invention during 1 hour. This is followed by a 24 hours infection period with the recombinant and non-replicative HIV virus coding for the firefly luciferase (Bona et al., 2006, Antimicrob. Agents Chemother. 50: 3407-3417). The inhibitory effect of the tested compound on virus entry in MAGI R5 cells is measured by a reduction of luciferase signal (TopCount-NXT reader (Packard) and detection luciferase kit: steadylite HTS assay kit (Perkin Elmer)) in the presence of the compound of the invention relative to the maximum signal obtained from cells infected with the virus without any added compound. The results of the tested compounds are reported as the concentration of compound required to inhibit 50% (IC50) of the maximum luciferase signal.

When tested in the assay described above and by way of illustration the compounds no 24, 64, 65, 25, 28, 1, 2, 7, 3 and 5 have an IC50 ranging from 25.6 nM to 2.5 μM (table 9)

### ¹²⁵I-MIP-1β Binding Competition Assay

The ability of the compounds of the invention to inhibit the binding of MIP-1β was assessed by an in vitro radioligand binding assay. Membranes were prepared from Chinese hamster ovary recombinant cells which express the human CCR5 receptor. The membranes were incubated with 0.05 nM ¹²⁵I-MIP-1β in a HEPES 25 mM/CaCl₂ 5 mM/MgCl₂ 1 mM buffer and various concentrations of the compounds of the invention. The amount of iodinated MIP-1β bound to the receptor was determined after filtration by the quantification of membrane associated radioactivity using the TopCount-NXT reader (Packard). Competition curves were obtained for compounds of the invention and the concentration of compound which displaced 50% of bound radioligand (IC50) was calculated

According to the method described above and by way of illustration the compounds no 24, 64, 65, 25, 28, 1, 2, 7, 3 and 5 have an IC50 ranging from 0.14 nM to 158.2 nM (table 9)

### Results

### 1. Activity of Compounds of the Invention

TABLE 9
Activity of compounds of the invention

| | Aequorin/Ca⁺⁺ | ¹²⁵I-MIP-1β | |
|---|---|---|---|
| | assay | binding assay | HIV-Infection assay |
| Compound | EC50 (nM) | IC50 (nM) | IC50 (nM) |
| 24 | 104.03 | 3.25 | 383.73 |
| 64 | 83.36 | 0.14 | 25.63 |
| 65 | 117.44 | 0.64 | 71.73 |
| 25 | 90.77 | 67.49 | 1132.22 |
| 28 | 619.67 | 158.17 | 2543.22 |
| 1 | 58.82 | 9.32 | 333.29 |
| 2 | 33.20 | 6.61 | 344.69 |
| 7 | 43.24 | 26.69 | 1420.64 |
| 3 | 27.86 | 14.81 | 871.28 |
| 5 | 144.64 | 23.40 | 425.07 |
| 81 | 38.1 | 0.2 | 14.6 |
| 90 | 180.2 | 0.7 | 5.3 |
| 93 | 50.8 | 0.5 | 1.9 |
| 97 | 94.4 | 0.2 | 3.0 |
| 80 | 31.3 | 0.8 | 27.0 |
| 113 | 29.4 | Not tested | 4.6 |

The aequorin-based assay quantitatively determines if the compounds exhibit agonist activity by inducing activation of the CCR5 receptor. The values mentioned in the Table 9 clearly indicate that this is the case. Indeed these values show that the compounds of the invention are able to activate the CCR5 receptor and therefore exhibit agonist activity.

The results of the inhibition of MIP-1β (a reference CCR5 ligand) binding assay represented in table 9 evidences that the compounds of the invention are able to specifically and competitively interact with CCR5 receptor.

In addition to the above-mentioned functional and binding activities on the CCR5 receptor, the compounds of the invention are also able to protect a human recombinant cell line (MAGI R5 cell) from the infection by a recombinant HIV virus (see table 9, column HIV-Infection assay), which is known to correlate closely with infection of human leukocytes with pathological strains of HIV

In other words the above-mentioned results demonstrate that the compounds of the invention are of value in inhibiting the entry of HIV viruses into target cells and therefore are of value in the prevention of infection by HIV viruses, the treatment of infection by HIV viruses and the prevention and/or the treatment of acquired immune deficiency syndrome (AIDS).

2. Transition from Antagonist to Agonist Activity

TABLE 10

| | Aequorin/Ca⁺⁺Agonist | Aequorin/Ca⁺⁺Antagonist |
|---|---|---|
| | assay | assay |
| Compound | EC50 (nM) | IC50 (nM) |
| 1 | 58.82 | Inactive |
| 61 | Inactive | 34.92 |
| 31 | 470.10 | Inactive |
| 63 | Inactive | 1430.64 |
| 37 | 4252.08 | Inactive |
| 62 | Inactive | 1229.82 |

It has surprisingly been observed that the substitution of the methyl group by a hydrogen at the amide bond nitrogen introduces a switch from an antagonistic mode of action to an agonistic mode of action of the compounds of the invention. To explain the different modulation pattern, Molecular Modeling was applied on the compounds 1 and 61 (Table 10). Geometrical comparisons of all possible low-energy conformations for the agonist and antagonist compounds were used to study the bioactive conformation retrieved by Molecular Modeling, i.e. for the conformation presumably adopted by the agonistic and antagonistic modulators during interaction with CCR5 receptor (FIG. 1A/B and FIG. 2A/B). A non hydrogen substitution of the amide bond nitrogen introduce specific sterical strains between a given substituent and the piperidine ring, compared to the non substituted amid bond nitrogen. Our studies show that due to these sterical strains the antagonistic modulating molecules are not able to retain the bioactive conformation of the agonistic modulating molecules. The generated computational models show a key interaction between the agonistic modulating compounds and the W6.48 (following the Ballesteros Weinstein Notation System: Ballesteros J. A. and Weinstein H., Methods Neuroscience (25), 1995, 366-428). The W6.48 acts as a switch between the presumably active and inactive receptor conformation (Ruprecht J. J., Mielke T., Vogel R., VIIIa C. and Schertler G. F., EMBO J. (23), 2004, 3609-3620; Springael J. Y., de Poorter C., Deupi X., Van Durme J., Pardo L. and Parmentier M., Cell Signal. (19), 2007, 1446-1456; Govaerts C., Bondue A., Springael J. Y., Olivella M., Deupi X., Le Poul E., Wodak S. J., Parmentier M., Pardo L. and Blanpain C., J Biol. Chem. (278), 2003, 1892-1903; Fano A., Ritchie D. W. and Carrieri A. J Chem Inf Model. (46), 2006, 1223-1235). This key interaction cannot be satisfied by any low-energy conformation of the antagonistic modulating compounds.

By way of illustration two other pairs compounds (agonist vs. antagonist) were synthesized and tested. As shown in Table 10 the two other agonist modulators (compounds no 31 and 37) are changed to antagonist modulators by N-methylation (compounds no 63 and 62). The functional results showed in table 10 are reported as EC50 (agonistic mode of action) or IC50 (antagonistic mode of action) as described in the Aequorin-based assay mentioned in the Biological Examples of this application (Inactive means >30000 nm).

1. A compound selected from the group consisting of 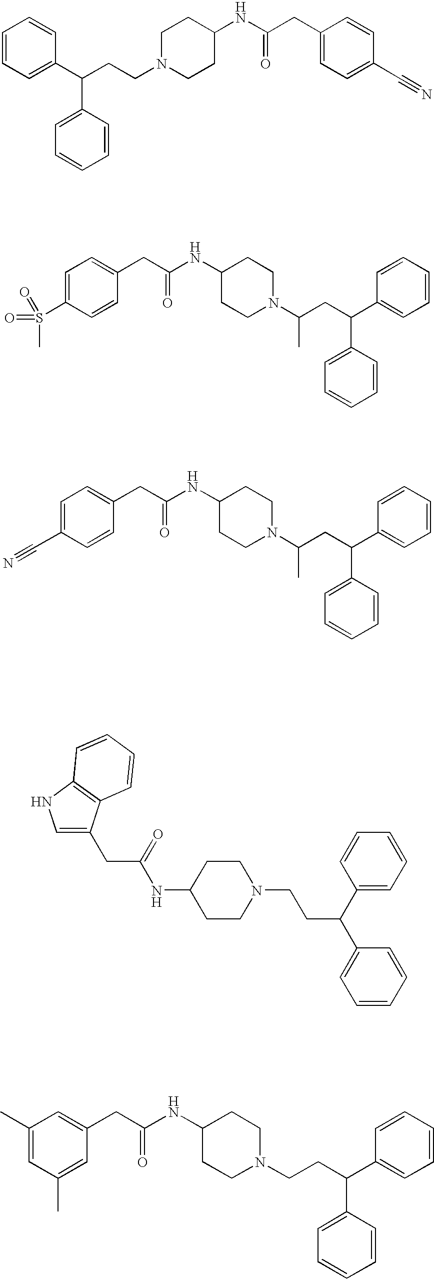 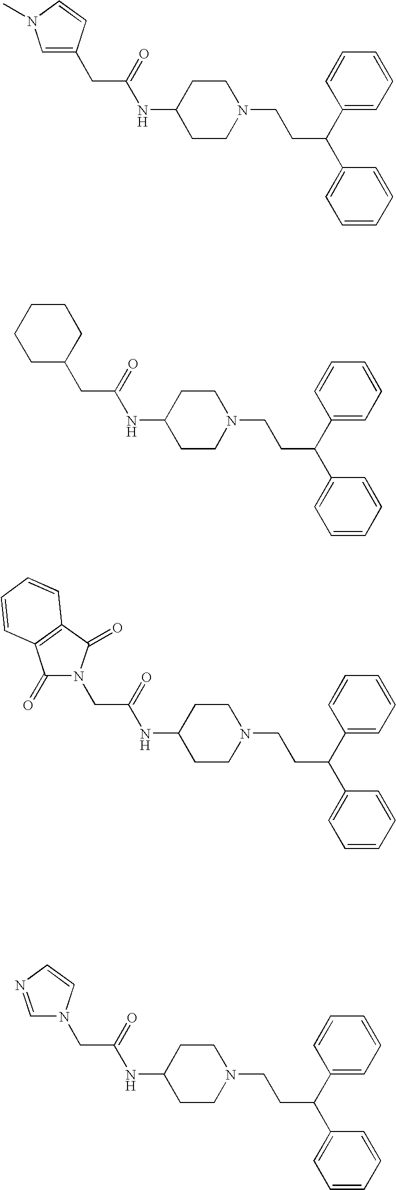 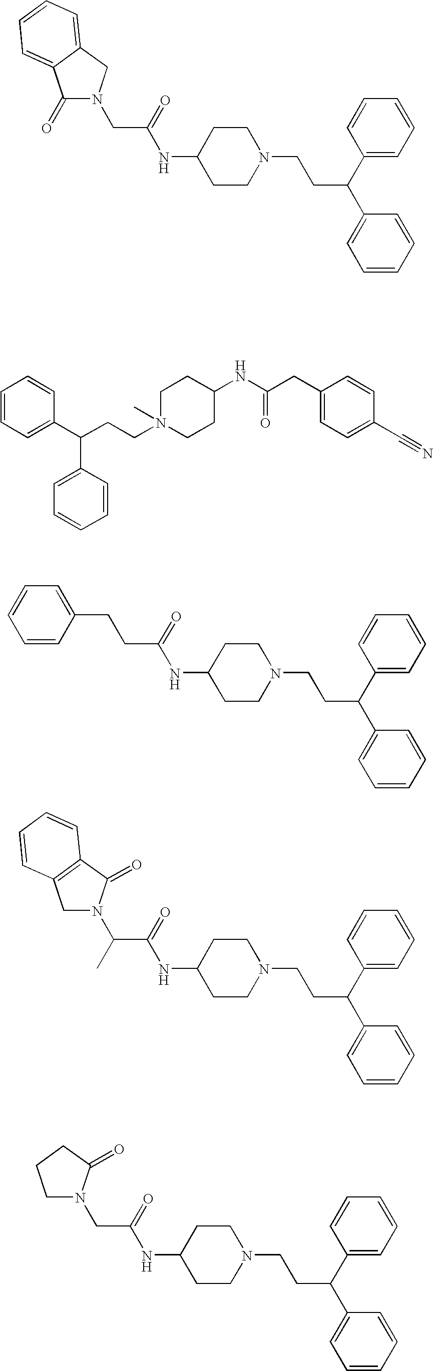 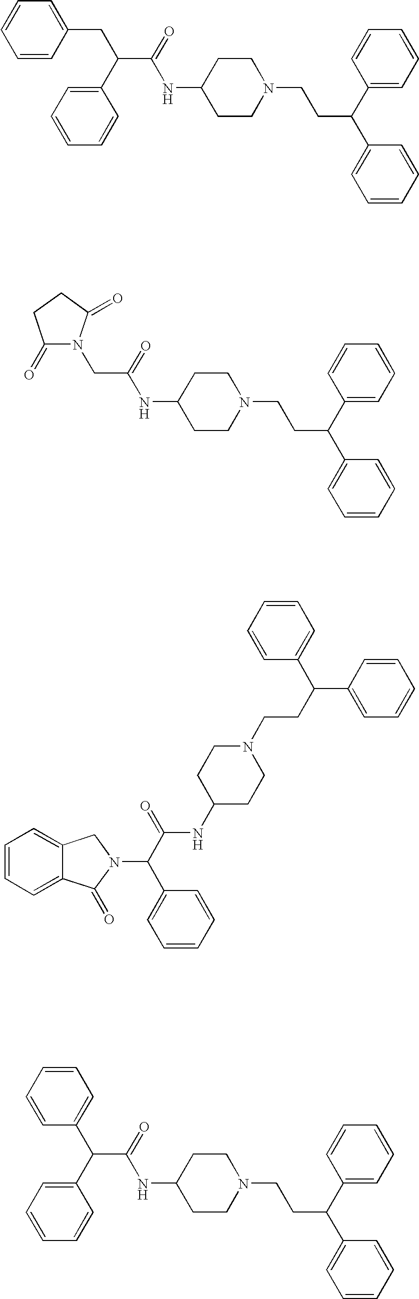 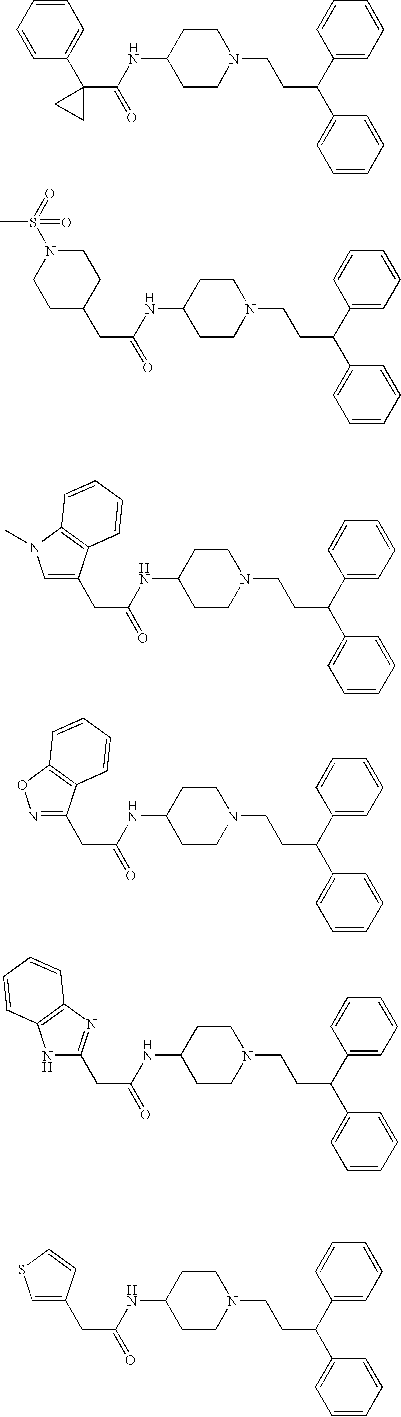 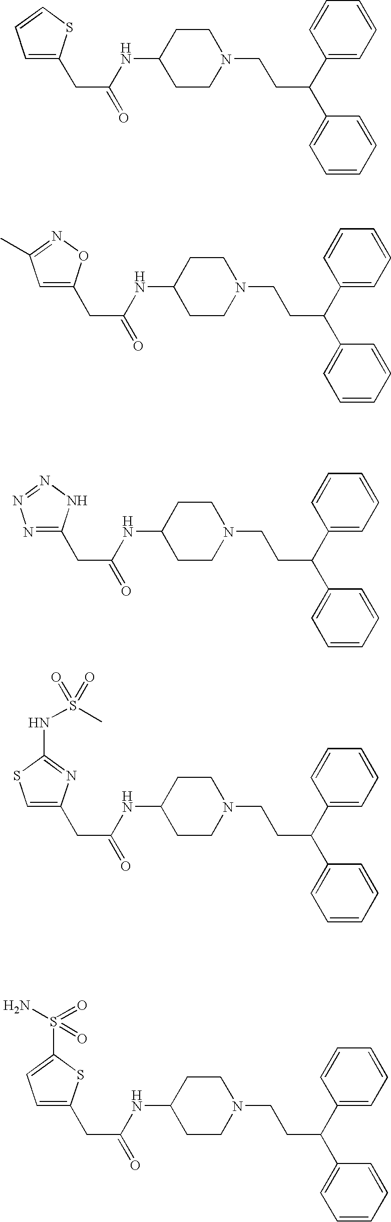 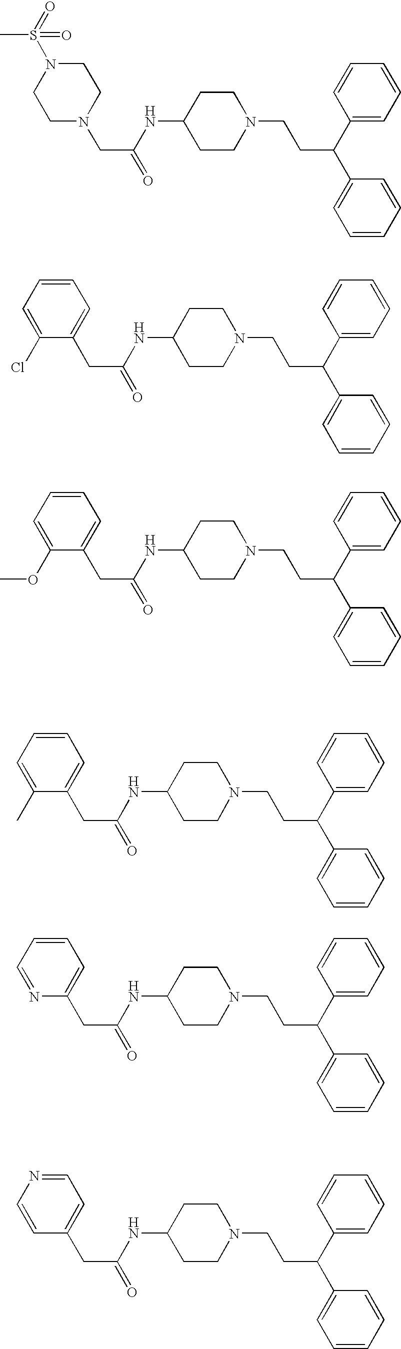 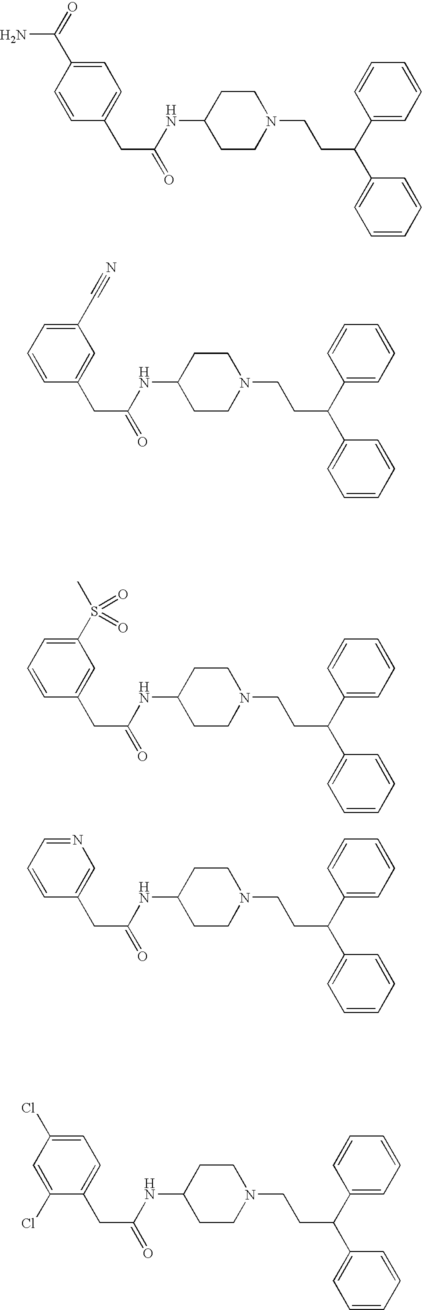 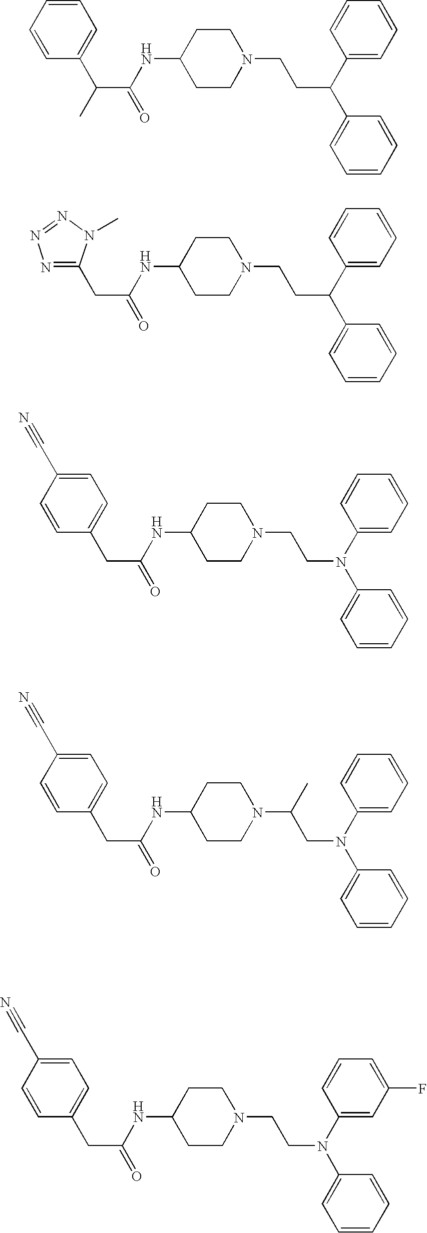 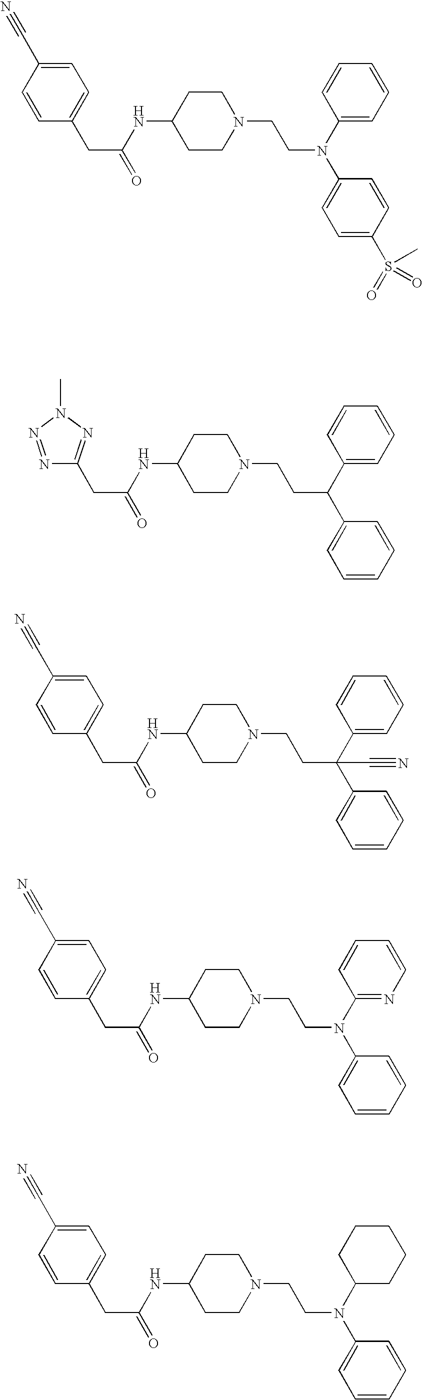 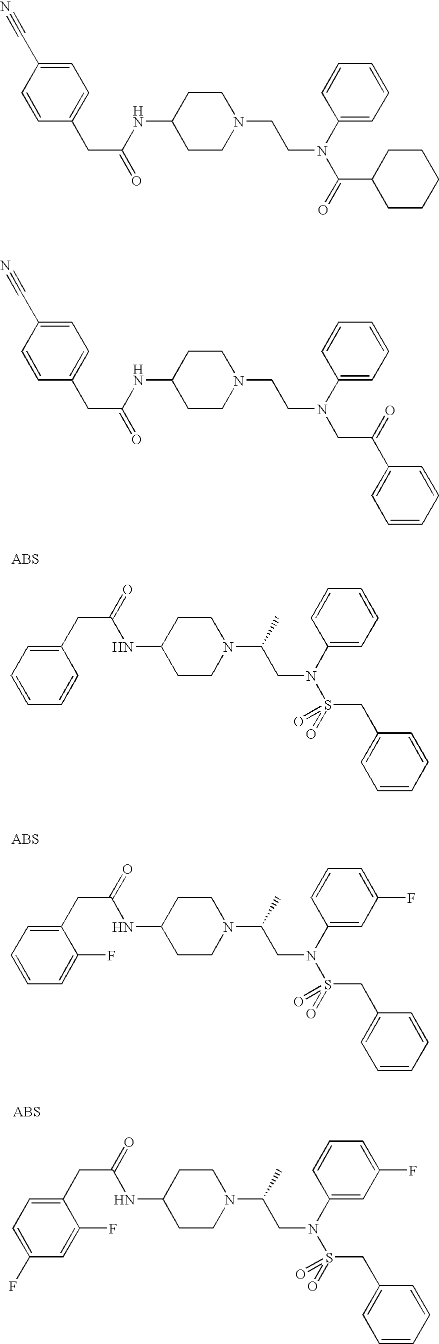 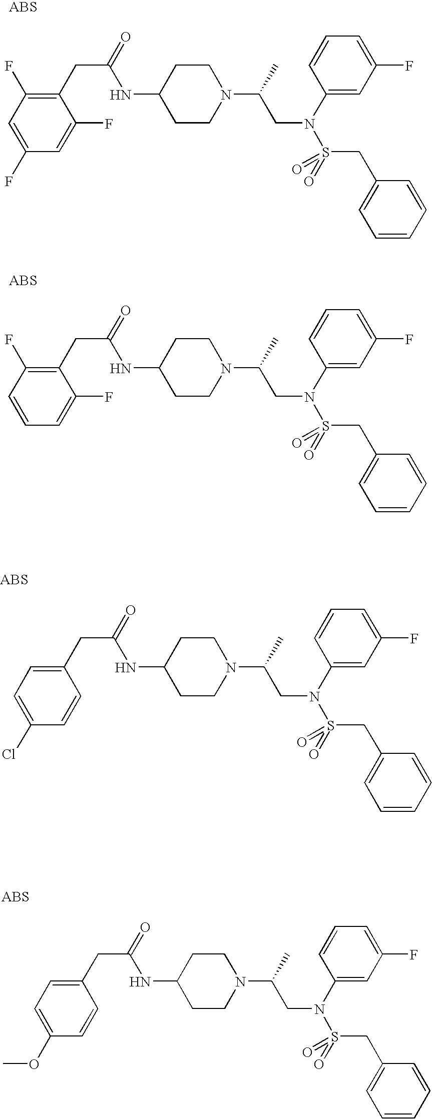 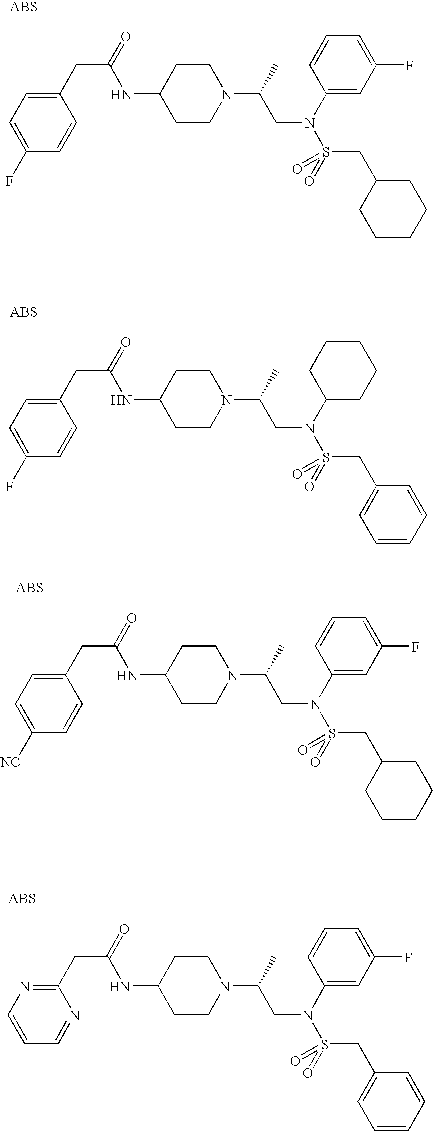 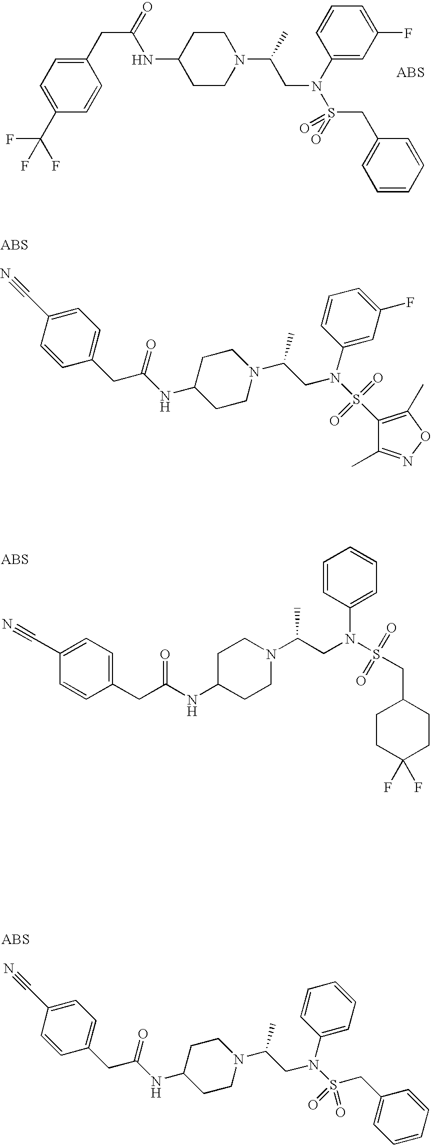 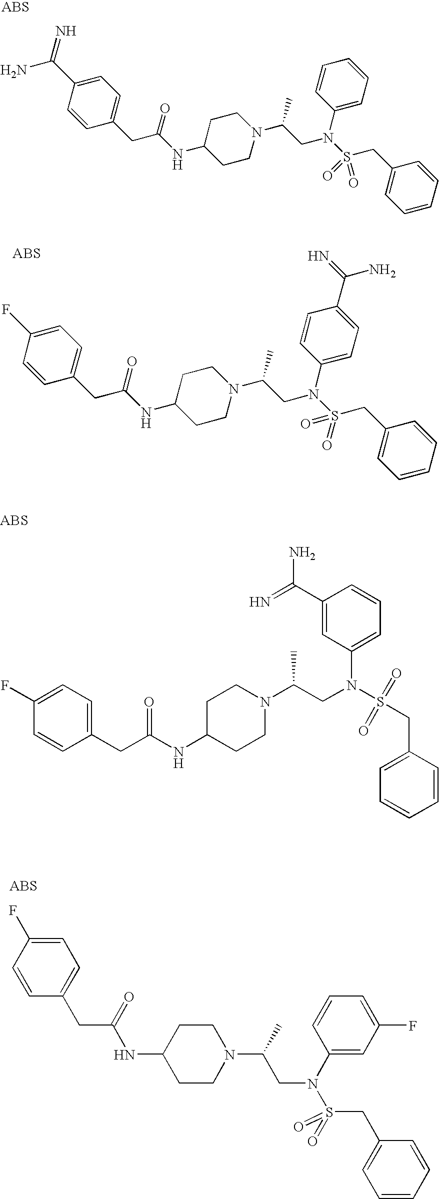 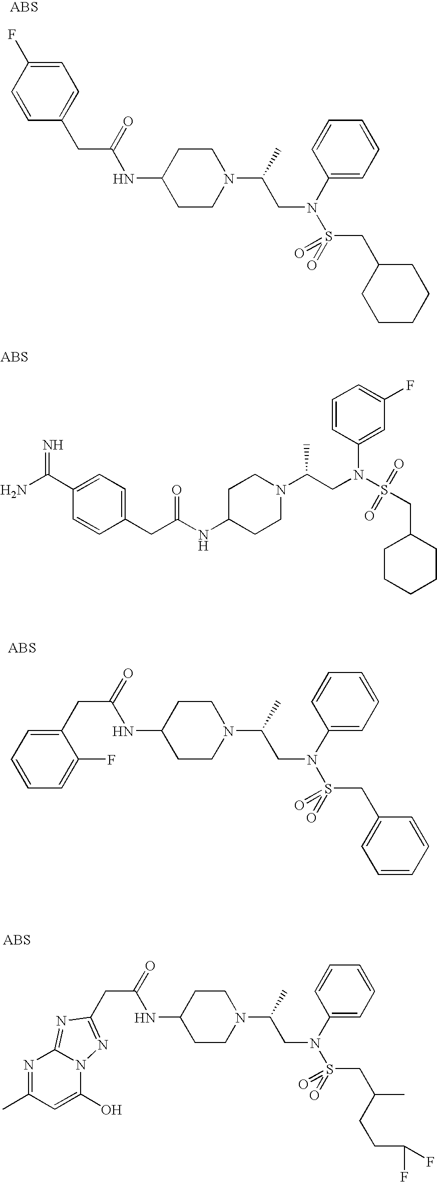 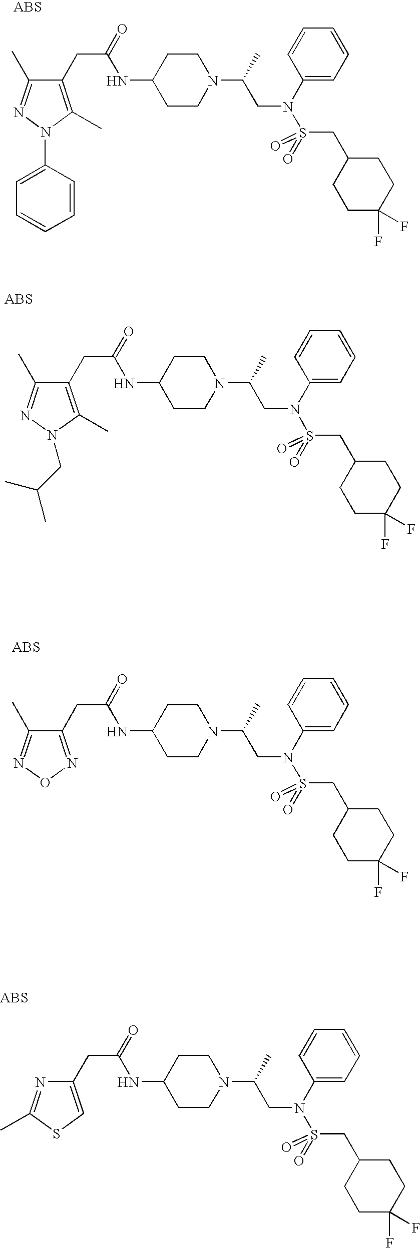 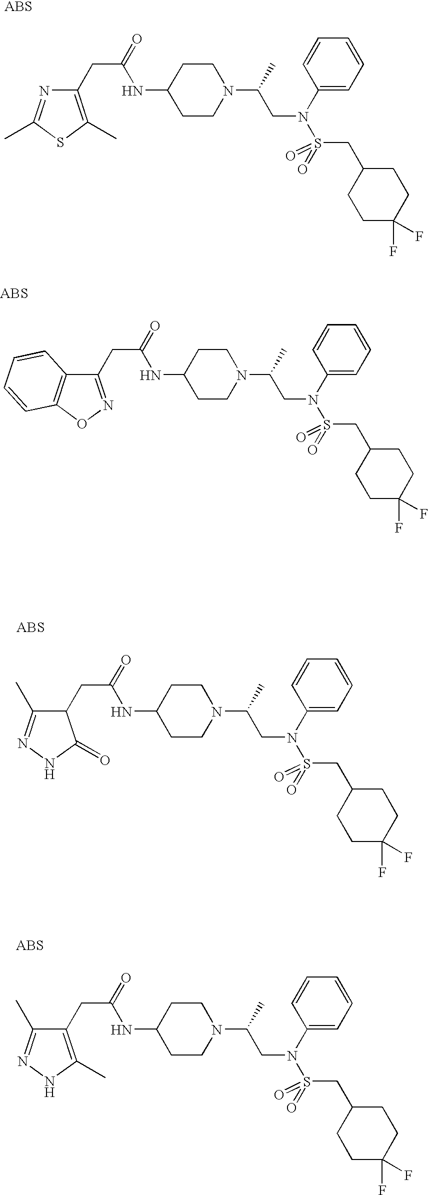 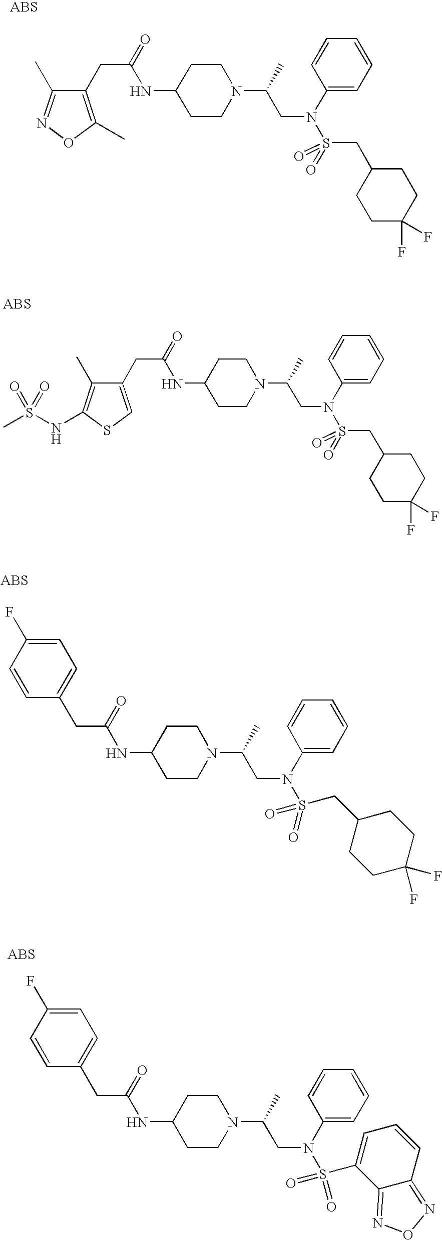 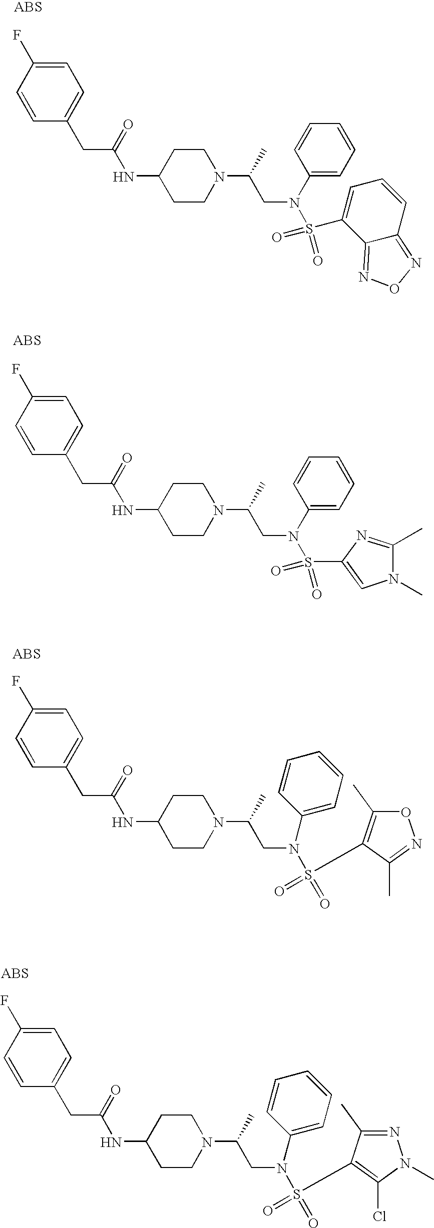 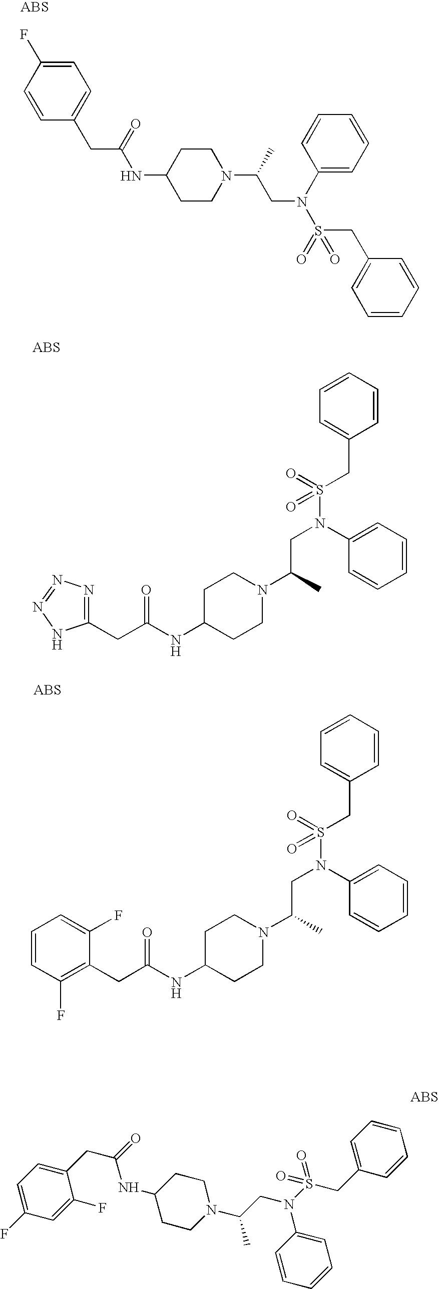 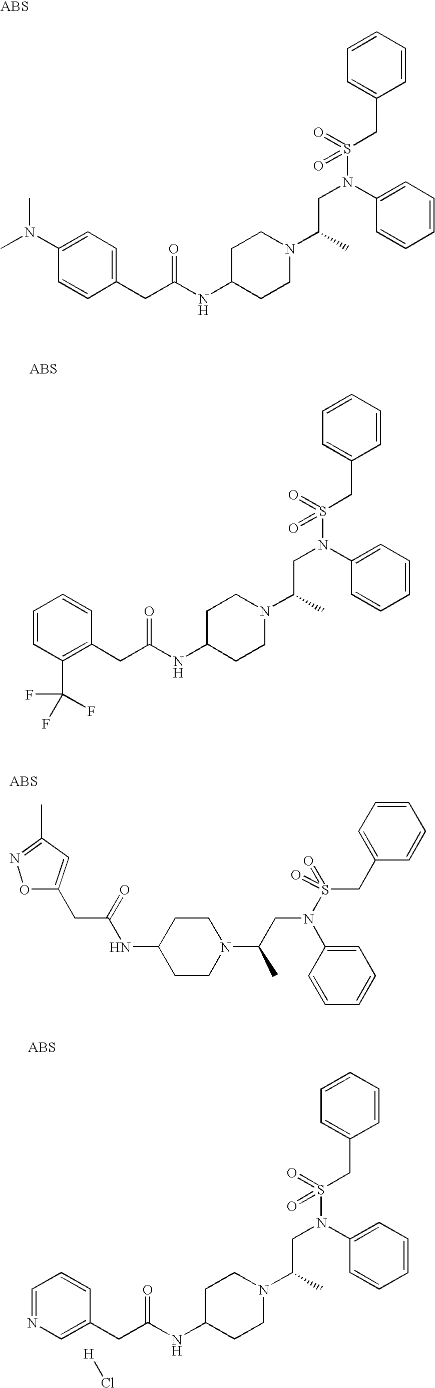 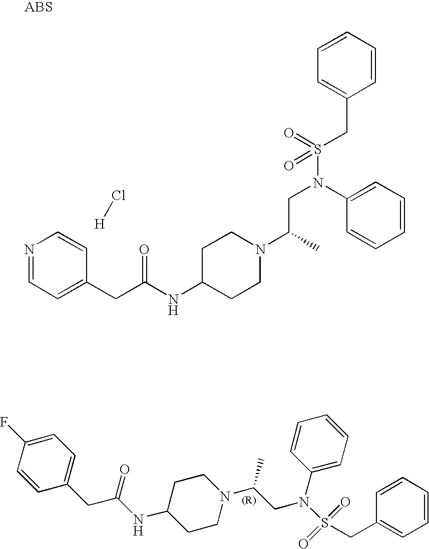
and its pharmaceutically acceptable salts and solvates.

2. An agonist of chemokine receptor activity comprising formula I 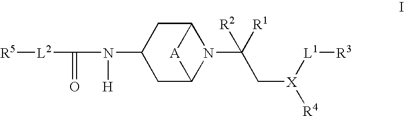
and pharmaceutically acceptable salts and solvates thereof, wherein
A is —CH₂—CH₂— or absent;
R¹ and R² independently are H, halo, optionally substituted alkyl, aryl, heteroaryl, cycloalkyl, cycloalkylalkyl, heterocyclyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
L¹ is CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;
X is CR⁶ or N;
R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said substituents being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
R⁶ is selected from hydrogen, hydroxyl, alkoxy, carboxy, CO—NR¹⁰R¹¹ halo, C₁-C₆ alkyl, cyano, and allyl;
R¹⁰ and R¹¹ independently are H, alkyl, or cycloalkyl; and
L² is a single bond or C₁-C₄ alkylene, optionally substituted by one or more substituent(s) selected from halo, oxo, nitro, cyano, azido, alkyl, hydroxyalkyl, haloalkyl, cycloalkyl, and alkoxy, or L² is CR^{a}R^{b}, wherein R^{a} and R^{b} form together with the carbon to which they are attached a carbocycle having 3 to 6 ring atoms.

3. The agonist according to claim 2, wherein
R³, R⁴, R⁵, and L¹ are defined as in claim 2,
A is absent;
R¹ and R² independently are hydrogen, halo, or C₁-C₄ alkyl;
X is CH, C(OH), C(CN), or N; and
L² is CH₂.

4. The agonist according to claim 3, wherein
A is absent;
R¹ is methyl;
R² is hydrogen;
X is CH or N;
L¹, R³, R⁴, and R⁵ are defined as in claim 2; and
L² is CH₂.

5. The agonist according to claim 2, wherein the compound has formula Ia 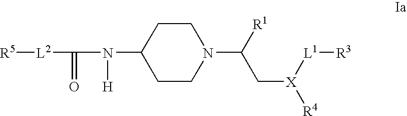
and pharmaceutically acceptable salts and solvates thereof, wherein
R¹ is hydrogen or methyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl, or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
X is CR⁶ or N, preferably CH or N;
R⁶ is selected from hydrogen, hydroxyl, alkoxy, carboxy, halo, C₁-C₆ alkyl, CO—NR¹⁰R¹¹, cyano, and allyl;
R¹⁰ and R¹¹ independently are H, alkyl, or cycloalkyl;
R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
L¹ is CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;
L² is defined as in claim 2; and
R⁷ and R⁸ independently are hydrogen, halo, optionally substituted alkyl, aryl, heteroaryl, cycloalkyl, or heterocyclyl, or R⁷ and R⁸ form together with the carbon atom to which they are connected a 3 to 6 membered saturated or unsaturated cycle.

6. The agonist according to claim 2, wherein the compound has formula Ib 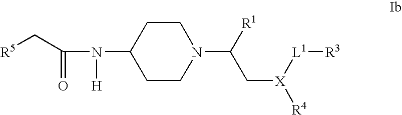
and pharmaceutically acceptable salts and solvates thereof, wherein
X is CH or N;
R¹ is hydrogen or methyl;
L¹ is CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a halcalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
R⁵ is defined as in claim 2.

7. The agonist according to claim 2, wherein the compound has formula Ic 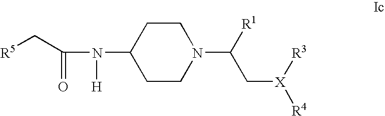
and pharmaceutically acceptable salts and solvates thereof, wherein
X is CH or N;
R¹ is hydrogen or methyl;
R³ is defined as in claim 2;
R⁴ is phenyl or pyridinyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group; and
R⁵ is defined as in claim 2.

8. The agonist according to claim 2, wherein the compound has formula Id 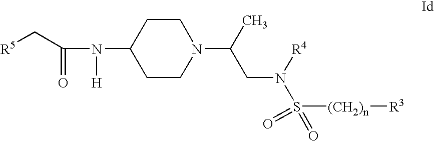
and pharmaceutically acceptable salts and solvates thereof, wherein
n is 0, 1 or 2;
R³ is aryl, heteroaryl or cycloalkyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group;
R⁴ is defined as in claim 2; and
R⁵ is defined as in claim 2.

9. The agonist according to claim 8, wherein the compound has formula Id′ 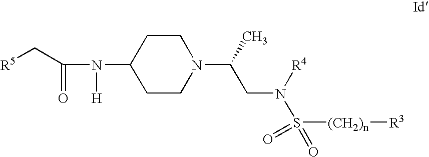
and pharmaceutically acceptable salts and solvates thereof, n, R³, R⁴, and R⁵ being defined as in claim 8.

10. The agonist according to claim 2, wherein the compound is a compound according to claim 1 or is selected from the group consisting of 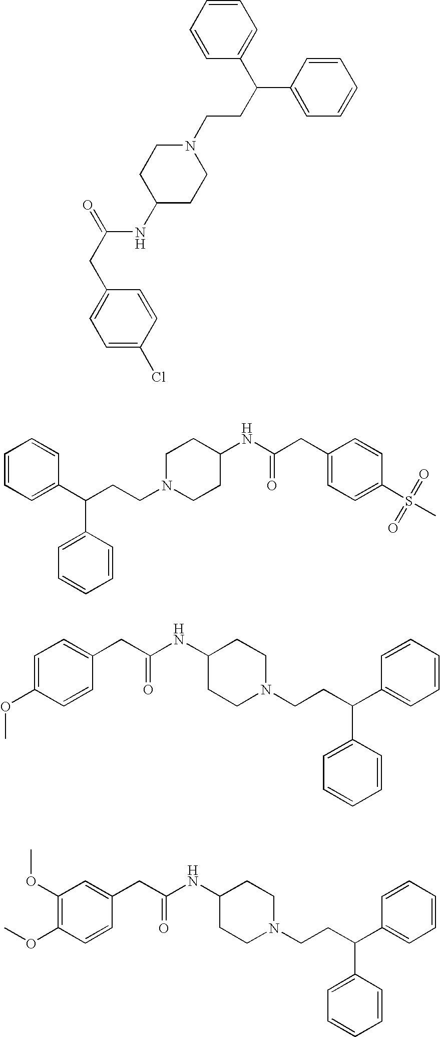 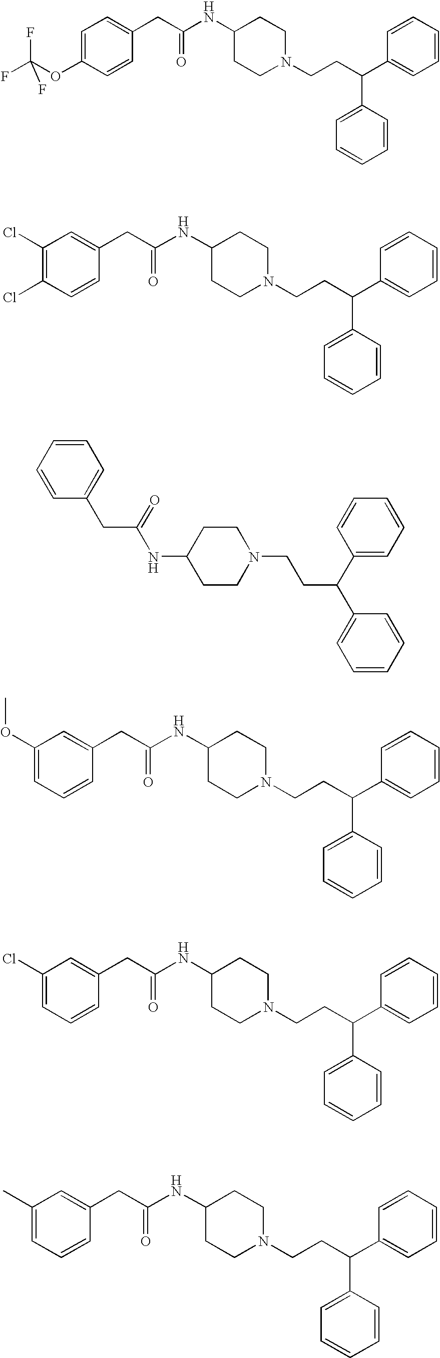 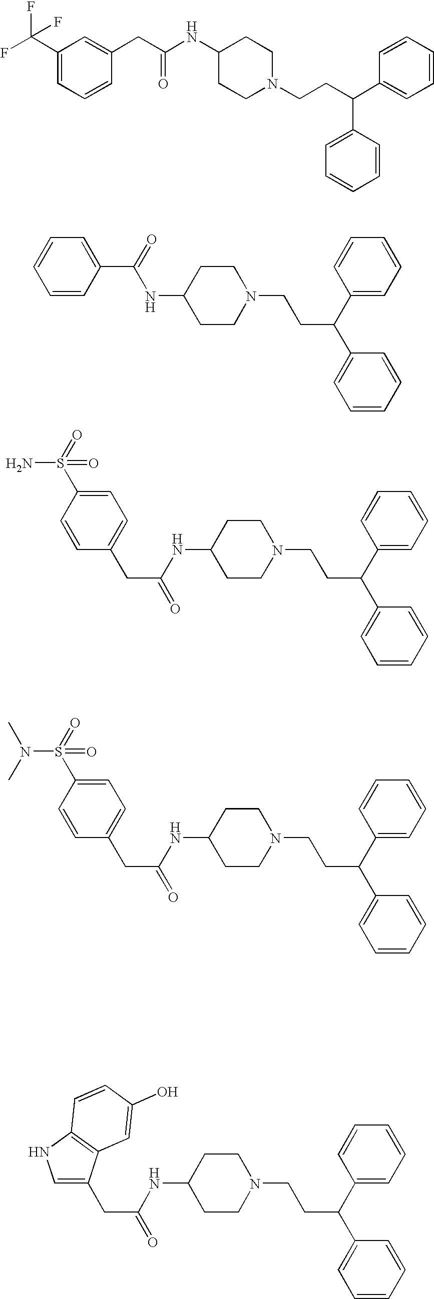 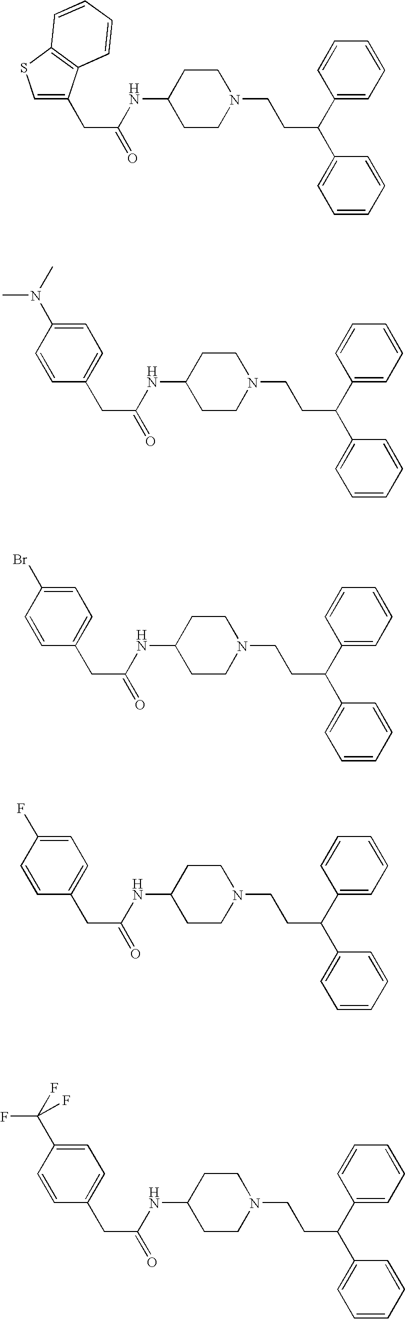 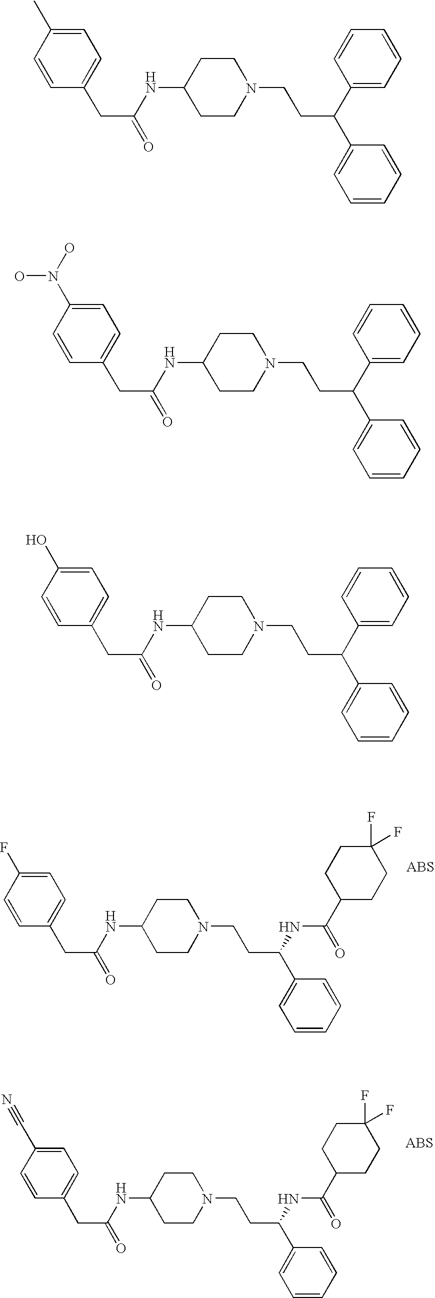 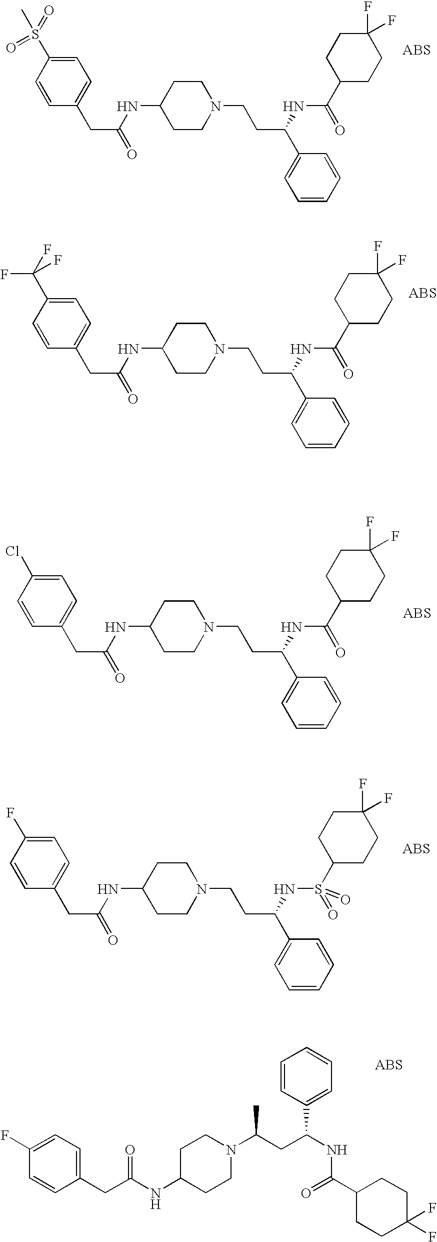 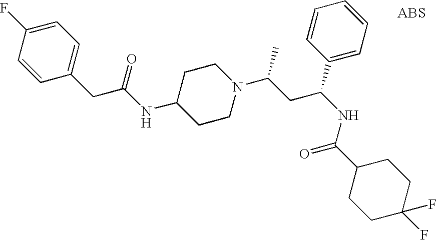
and pharmaceutically acceptable salts and solvates thereof.

11. The agonist according to claim 2, wherein the chemokine receptor is a CC chemokine.

12. The agonist according to claim 11, wherein the chemokine receptor is CCR5.

13. A pharmaceutical composition comprising a compound according to claim 1 or a pharmaceutically acceptable salt or solvate thereof and at least one pharmaceutically acceptable carrier, diluent, excipient and/or adjuvant.

14-24. (canceled)

25. A method of treating and/or preventing autoimmune inflammatory, infectious, proliferative or hyperproliferative diseases, or immunologically mediated diseases, comprising administering a therapeutically effective amount of a compound of formula I as defined in claim 2 as agonist of chemokine receptor activity, or of a compound according to claim 1 or a pharmaceutically acceptable salt or solvate thereof or a pharmaceutical composition according to claim 13 to a patient in need thereof.

26. The method according to claim 25, wherein the disease is selected from AIDS, inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary diseasemetastatic cancers and renal diseases.

27. The method according to claim 26, wherein the disease is AIDS caused by HIV-1 or -2 infection.

28. A method of inhibiting the entry of viruses into target cells and, therefore, for the prevention of infection by viruses, the treatment of infection by viruses, comprising administering a therapeutically effective amount of a compound of formula I as defined in claim 2 as agonist of chemokine receptor activity, or of a compound according to claim 1 or of a pharmaceutically acceptable salt or solvate thereof or a pharmaceutical composition according to claim 13 to a patient in need thereof.

29. The method according to claim 28, wherein the virus is human immunodeficiency virus.

30. The method according to claim 29 for the prevention and/or treatment of acquired immune deficiency syndrome.

31. A method of modulating chemokine receptor activity in a patient comprising administering a therapeutically effective amount of a compound according to claim 1 or of a compound of formula I as defined in claim 2 as agonist of chemokine receptor activity, or a pharmaceutically acceptable salt or solvate thereof or a pharmaceutical composition according to claim 13 to a patient in need thereof.

32. The method according to claim 31, wherein the chemokine is CCR5.

33. The method according to claim 25, wherein the compound according to claim 1 or the compound of formula I or the pharmaceutically acceptable salt or solvate thereof or the pharmaceutical composition is administered in combination with at least one additional therapeutic agent and/or active ingredient.

34. The method according to claim 28, wherein the compound according to claim 1 or the compound of formula I or the pharmaceutically acceptable salt or solvate thereof or the pharmaceutical composition is administered in combination with at least one additional therapeutic agent and/or active ingredient.

35. The method according to claim 31, wherein the compound according to claim 1 or the compound of formula I or the pharmaceutically acceptable salt or solvate thereof or the pharmaceutical composition is administered in combination with at least one additional therapeutic agent and/or active ingredient.

36. A pharmaceutical composition comprising the agonist of claim 2 or a pharmaceutically acceptable salt or solvate thereof and at least one pharmaceutically acceptable carrier, diluent, excipient and/or adjuvant.

37. A method of treating and/or preventing autoimmune, inflammatory, infectious, proliferative or hyperproliferative diseases, or immunologically mediated diseases, comprising administering a therapeutically effective amount of the pharmaceutical composition according to claim 36 to a patient in need thereof.

38. A method of inhibiting the entry of viruses into target cells and, therefore, for the prevention of infection by viruses, the treatment of infection by viruses, comprising administering a therapeutically effective amount of a pharmaceutical composition according to claim 36 to a patient in need thereof.

39. A method of modulating chemokine receptor activity in a patient comprising administering a therapeutically effective amount of a pharmaceutical composition according to claim 36 to a patient in need thereof.
